SEQUENCE LISTING (23 sequences)

SEQ ID NO: 1 - DNA, 47 bp, Artificial Sequence
  synthetic oligonucleotide
ctttccctacacgacgctcttccgatctgtgccagcmgccgcggtaa

SEQ ID NO: 2 - DNA, 48 bp, Artificial Sequence
  synthetic oligonucleotide
ggagttcagacgtgtgctcttccgatctggactachvgggtwtctaat

SEQ ID NO: 3 - DNA, 49 bp, Artificial Sequence
  synthetic oligonucleotide
aatgatacggcgaccaccgagatctacactctttccctacacgacgctc

SEQ ID NO: 4 - DNA, 49 bp, Artificial Sequence
  synthetic oligonucleotide
caagcagaagacggcatacgagatgtgactggagttcagacgtgtgctc

SEQ ID NO: 5 - DNA, 1569 bp, Veillonella atypica
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggatcagttagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctcctttctagggaga

SEQ ID NO: 6 - DNA, 1569 bp, Veillonella dispar
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgaggatagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcaataccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctcctttctagggaga

SEQ ID NO: 7 - DNA, 1569 bp, Veillonella parvula
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagttagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctcctttctagggaga

SEQ ID NO: 8 - DNA, 1356 bp, Veillonella atypica
atggaaacaagaattgaatatgattcaatgggaccagtcgaagtcgacgctagacgaatttacggacctcaaacgcaacgctcgttcaataactttaagatcggtgaccaccgcatccctattgaacaaatcaaagcgcttgcgcttgtcaaaaaagcatgtgctttaaccaatgcaaaatgtggcgcagtaactgaggaaaaagcgaaactcattgcccaagtagtagatgaaatcgtagatggcaaatgggatgaggaattcccattgaccgtattccaaacaggctctggcacacaaacaaacatgaatgtgaacgaagttatcgctcaccgggctaagcagttagatgaagctaatccacttcatcctaacgacgatgtaaaccgcggccaaagtacaaatgatacattcccaacagcaatgcatatctgcgggtactttgaaattacaaaacgcgtaattcctgcattggacggccttattgtatcctttgaaaaattgcaagaaaaaggtataggcttgcaaaaggttggtcgtactcacctacaagatgctactttcatcatggtggatcaagaaattagcgcctttgtagacggcctaaaaactgccaaaactatgctccttcaaaacgctgactacctactcgacgttgcccttggcggcactgccgttggtacaggtgtgaacacacctaagggatatctagacgttatggaaaccgtattgccagaagtaacaggcgctccattccgcgttaagaataataaattccaaggcctatccttaaaagatgcgttcatgatggctcacggcgcacttaacacattggctactacattgtttaagattgcgaacgatgtccgtttcttaggatccggccctcgctgtggctatggcgaatggcatcttcctgaaaacgaaccaggctcctccatcatgccaggtaaggttaaccctacccaatgcgaagctcttgctatggtatgtgcccaagtattcggtatcaatacaaccatgacactttgtgcaggtagcggtgcgttccaattgaacgtgtacatgcctatcatgatctatgattttgttgaaagctgtcgcctcctcgcagatgcgatgaattccttcactacacactgcatcgacggcgtagaattcgtacctgaaaaacttaaattcttcgtagaacaatcccttatgattgctacttccttaacaccatacatcggctacgataagagtgctaaggtcgtaaaagaagcatacaaacgcggttgctctattaaagaaatcatcttggaagaaaaactcatgaccgaagacgaatttgctgaagcagttcgcatgaaataa

SEQ ID NO: 9 - DNA, 843 bp, Veillonella atypica
ttgcgcgagattcaagtatctgaaatcacaaaaacagtccgtcaaatgtgtatggacgcagcttaccacttgccaaaagacatctatgaaggcttgaaaaaaggccgtgaaacagaagagtctccagttggttgcatcgttctcgatcaaatcatcaaaaatgcagaaattgctgatgctgaagatcgtccatactgccaagatactggtatgactatggtattcttaaaagttggtcaagatgttcatttcgttggtggtgatcttacagaagctatcaatgctggtgttgcagctggttatgttgaaggttatcttcgtaaatccgttgtagctgaaccattgttcaatcgtaaaaatacacaaaataacacacctgcaatcatctatactgaaatcgttcctggcgataaagtagatattcaagtagaattaaaaggtttcggttctgaaaataaatccgatgtagctatgctcgtacctgctgatggtgtggaaggcgttaaaaatgcagtccttgaaatcgtaaaacatgcaggtcctaacccatgccctccaattgtactcggcattggtattggcggtactatggaccaagcagctgttatgtccaaaaaagcgttgcttcgtgacattagcgtacctcataaagatgcagactatgcaaaattagaagaagaaatcatggaaatggttaacaaaactggtattggaccacaattgggtggcactacaacttgtatcggtgtaaacatcgaatggggtgcaactcacatcgcaggtcttcctgttgcagttactatcatgtgccatgctgctcgtcatgctcacgtagtactttaa

SEQ ID NO: 10 - DNA, 963 bp, Veillonella atypica
atgagacaaatcacctatcatatccaccgttaccaacaaggtcgcgcgtttgtacaaactttcaaattcgactatgaagctgaccgtacaattctttggggccttcaaaaaattaaagatactcaagatccaacattaacattcttggcagcttgtcgttccgcagtttgtggcgcttgctccattcgcgtaaatggcgaagcaatgcttggttgtgaatctaaaattgatgaattgacagaacgttatggtacagatgaattaactatcgctcctatcggtaacttccgtgtaatccgcgacttagttgttgattgggaatctaaagttgatcgtttgaaaacagttgctccttggattttccttaaagcagaattcaacgaaggcgacaaaatcgttcgtcaaactccagctgatttcaaaaaattcgttgctggtacagaatgtatcctttgcggttgctgcgcatctgaatgtaacaaattgactgcacgtcaagacgatttcttggaaccatatgtattcactaaagctaaccgtttcgtattggatagccgtgatgatgcacctatggctcacattcaacctgcttatgacaatggtctttggaaatgcgttcactgcatgaactgtatctcccgttgtcctaaacacttaaaacctgctcaagatatttccaacatgcgtaaagaagctacaaaagctggccttacaaacaacaaaggcgttcgccatgcagttgctttcaaagaagacctttacaaaactggtcgtttgaaagaagtttctatgagcttgaaatctgacggtgttgtagattccgctaaacaagcattctatgcattacgtttgtggaaacacagcaaaattaatcctttcgaacttgtagtacctcaaaaaccagttaatggtattgatggtgttcgcagacttatgaaagcagctgaggaggtaagcaaataa

SEQ ID NO: 11 - DNA, 948 bp, Veillonella atypica
atgaaattacgtaaagtaggtattatcggaactggacatgtaggctcgcatgtagccttttctttagctttgcaaggcgaagttgatgagttatatatgatggatattgatgaaaagaaagctaaagcacaggctatggatgttaatgatgcggttagttatatacctcatcgtgtaacggctacatctggtccgattgaagactgtggtgattgtgatattcttgtttttagtgcaggtccactacctaatttatatcaagatcgcctagaaagccttggtgatacaatcgcagtactaaaggatgttattcctcgcattaaagcatctggatttaagggctttattatttctatatctaatccagcagatgtagtggctacgtatttatgtaaacatttagattggaatccaaagcgcattatttcgtcaggtacagcgctagattctgcaagattgcaaaaagagttagctcatatattcgatattagcaatcgaactattactgcttattgcatgggtgagcatggcgcaagtgctatggtgccatggtcccatgtgtatgtacaaggcaagccgttagtagaattgcaaaaagaattgcctcatagatttccagaactagatcataaacaagtgttagatgatgttaaaattggtggatatcatgtgttggcaggcaaaggctctacggaatttggtatagcaagtgccaccacagagttaattcgctctgtattccatgatgagaaaaaagtattgccatgttcttgttatttagatggtcaatacggggaaacaggcgtttttgcatccacacctgctgtcattggcaaggatggcatagaagatgttcttgaattacaaatgacagaggatgaattggctttatttaagaaatcctgtgcggttattagagaatatgcaaaaaaagcagaaactatgtaa

SEQ ID NO: 12 - DNA, 1530 bp, Veillonella atypica
atgcaaacagtgcaagaaaaaattgagttgttgcacgaaaaactagcaaaagttaaagctggtggcggtgaaaaacgcgttgagaaacaacattctcaaggtaaaatgactgctcgtgaacgtttggctaaattattcgatgacaactcttttgttgaacttgatcaattcgttaaacatcgttgtgttaacttcggtcaagacaaaaaagaattaccaggcgaaggtgtagtaactggttatggtactatcgatggtcgtttggtatatgcattcgcacaagacttcactgtagaaggtggttctcttggtgaaatgcacgctgctaaaatcgttaaagtacaacgtttagcaatgaaaatgggtgctcctatcgttggtatcaatgattccggcggggctcgtattcaagaagctgtagatgctcttgctggttacggtaaaattttctttgaaaatacaaatgcatctggcgttattccacaaatttccgtaatcatgggaccatgtgcaggcggtgctgtatattctccagcattgactgacttcatctacatggttaaaaacacatctcaaatgttcatcactggtcctgcagttattaaatctgtaactggtgaagaagtaacagctgaagatcttggtggcgcaatggctcacaactctgtgtctggtgttgctcacttcgcagctgaaaatgaagatgattgcatcgctcaaatccgctacttgttaggtttcttaccttctaacaacatggaagatgctccattggtagatacaggtgatgacccaactcgtgaagatgaaggcttaaacagcttgttacctgataacagcaacatgccttacgacatgaaagatgttatcgcagctactgtagataatggcgaatactatgaagtacaaccattctatgctacaaacatcattacatgcttcgcacgttttgatggtcaatctgttggtatcattgctaaccaacctaaagtaatggctggttgcttggacatcaacgcatctgataaatcttcccgttttatccgtttctgtgatgctttcaatattccaatcgttaacttcgttgacgttcctggtttcttgcctggcacaaatcaagaatggggcggtatcattcgtcatggtgctaaaatgttgtatgcttactctgaagcaacagtaccaaaaattactgttatcactcgtaaagcatacggtggttcttacctcgctatgtgttcccaagatttgggcgctgatcaagtatacgcttggcctacatccgaaatcgctgtaatgggtcctgctggtgcagctaacatcatcttcaaaaaagatgaagataaagacgctaaaacagctaagtacgtagaagaattcgcaactccttacaaagctgcagaacgtggcttcgttgatgttgtaatcgaaccaaaacaaactcgtccagctgttatcaatgcgttggctatgcttgcaagtaaacgtgaaaaccgtgctccaaagaaacatggtaatattccattataa

SEQ ID NO: 13 - DNA, 423 bp, Veillonella atypica
atggcttttaaagtattacaagtggatcacatcggtattggtgttaatgatttagcagcaactaaagaattttacaaaaatgctttgggaattcaacatcttcctgaagatgaagtagtagaagaacaaaaagtaaaagtatccttcttcccatgcggcgatgctgaattagaattcttggaaactactactccagacggccctatcggtaaattcatcgaaaaaaatggcggtcgtgatggtatccaacacgttgctttgcgtgtagataatattgaaaatgctattgctgatcttatggcgaaaggtattcgtatgattgacgaaaaacctcgttatggtgctggcggttcctctattgcattcgttcatcctaaagctacaggtggcgtattacttgaactttgtcaacgaatgaaataa

SEQ ID NO: 14 - DNA, 2190 bp, Veillonella atypica
atgtttaaaaatccagacttctcctctcttggcttgggatctcagtctgccacatcgcgcgatgcatggcttgctgaacttaaaaaagaaacaggaaaaagctttgaagacttatataacacgacaatggagcaaatccaattaaaacctctctatacagagatggattatgaagggatgacacaccttgactatatggctggtgtacctccattcttgcgtggaccttattccacaatgtacgtaactcgtccttggacagtgcgtcagtacgctggtttctctacagccgaagaatccaatgcgttctatcgtcgtaacttggcagcaggccaaaaagggttgtctattgcctttgaccttgctactcaccgtggttatgactccgaccatcctcgcgtagtgggcgacgttggtaaagcgggcgttgcggtagactccatcctcgatatggaaattctattctctggtatccctcttgaccaaatgtccgtatccatgacaatgaacggcgccgtattgcctgttatggcattctacatcctagctggtgaagaacaaggtgttgataaaaaggttatggccggtacaatccaaaatgatatcttgaaagagttcatggtacgtaatacctatatttaccctccagctacgtccatgcgcatcatcggtgatatctttgcgtatacatcccagaacatgcctaaattcaacagtatctctatttctggctaccatatgcaagaagcgggtgcaacggcggatatcgaattaggttatacattggctgacggccttgaatacatccgtaccggtgtaaatgcaggtcttcatgttgaccaattcgcacctcgtttgtccttcttctgggctatcggcaaaaactactttatggaagtggctaaaatgcgtgcggctcgtatgttgtgggctaagattattaagagcttcggttctgaaaatcctaaatctatggctcttcgtacacatagccaaacatctggttggtctcttacagaacaagatccattcaacaacgtggctcgtacatgtatggaagcgatgggcgccgcattgggccatacgcaatccctacatacgaatgcgcttgatgaagccatcgcgttacctacagacttctctgcacgtattgcgcgtaatacacaactttacatccaagatgaaactaaggtatgtaaggttatcgacccatggggcggttcctactatgtagaagcattgactgacgaattgatccgtcgtgcctggggccatatccaagaaatcgaatcccttggtggtatggcgaaagcgattgacacgggtcttcctaagatgcgtatcgaagaggcggcagctcgtcgccaagctcgtatcgactctggtcgtgaagctatcgtcggcatcaataaatatcgtctagataaagaagatccattggatatcctcgatgtagataatacagctgtacgggaagcgcaaatccgtcgtctcgaacaattgcgtgctaaccgtgatgaagacaaggttcaatcctgcctcgaagcgattacaaatgctaccgaatctggtgaaggcaacttgcttgcccttgcacttgaagcagctcgtgctcgtgcatctttgggcgaaatttcctttgctgttgaaaaagtttgtggccgtcataaagcggttatccgctctatttccggtgtatactccagcgaatatgaagatgatgatgtaatcaaagaagtacgtcaaatggcagatgactttgaagaactcgaaggtcgtcgccctcgtatcatgattgctaagatgggccaagacggtcatgaccgcggtgccaaagttattgctacatccttcgcggatatgggctttgacgtggatatcggacctttgttccaaactccagaagaaacagcacaagatgcggtggataatgacgttcacatcgtcggatttagttctcttgcagcaggtcacaaaacattgttacctcaacttgtagaagaactcaacaagcgaggtcgtggagatattttagttgccatcggcggcgtaatccctgctcaggactatgagttcctacgcgaacatggcgcagtggctatctttggccctggtacagttttgccagtagcggcgaaaaaattactagaaacattgactagccacgttcaagacgaaggcaatgactga

SEQ ID NO: 15 - DNA, 3447 bp, Veillonella atypica
atgaagaaaattaaatccgttttggtagccaatcgtggcgaaatcgcaatccgcgtatttcgtgcatgtaacgaaatgggtattaaaacagtagctatctattctaaagaagacacattgtccttgcaccgtaaccaagctgatgaagcatatttggttggggaaggtaaaaaaccagttgatgcctatttggatattgaagatatcatccgcattgctaaagagcatgacattgatgctatccatcctggctatggtttcttatccgaaaacgaagagttcgctcgtcgttgcggtgaagaaggtatcattttcattggacctcacgtagaacatttgaatatgttcggcgacaaggttaatgctcgtacacaagctaaattggcagacattccaatgattccaggttctgatggggcattgcgtgattttgcacaattagaagaatttgctgaaactcacggctatccattgatgattaaagccgtaaacggtggtggcggtcgtggtatgcgcgaagttcaccgcaaagaagatttacgcgatgcttatgaccgcgctaaatccgaagctaaagcagcatttggtgatgacgatgtttacgtagaaaaactcatcgttgaacctaaacatattgaagtacaaatcttaggtgatgaacatggtaatgtagttcatttacatgaacgtgactgctctgtacaacgtcgtcaccaaaaggttgttgaaatggcaccagcttttgcattgccattagaaactcgtaaagccgtatgtgatgcagccgtaaaaatcatgaaaaatgttggctacgttaatgctggtactgttgaattcttggtaacttctgatggttccttctatttcatcgaagttaaccctcgtatccaagtagaacataccgtaacagaaatgattacggacatcgatattgttcattctcaaattcgtatcgctgaaggctatgatttgcacagcccagaaataggcattcctgaacaagatgaaattccttgtaaaggtactgcaattcaatgtcgtatcactacagaagatcctaagaacaactttatgccagatactggtaaaatcttagcatatcgtagttccggcggctttggtattcgccttgactctggtaatgctttcacaggcgctgtagtaacaccttattatgattccctattggtaaaagctactgcattcggtcctaacaacgaagaaactattcgtaaaatgcttcgttgcttgaaagaattccgtattcgtggcgttaaaacaaacattcatttcttgattaacgttcttgaaaaccctgaattccaaagcggtaactacacagttaacttcattgaagaccatcctgaattattcgaattaaaaccagatcgcgaccgtggcactaaattgcttcgctacattgcggatactacaatcaatggttactccggtgcaggccctcaagaggtacctgattttgaaccaatgcaattgccatctaaattagatgtatctcctgcacctggtacaaaacaaaaattcgacgaattaggtccagaaggtttcagtaaatggttggctgaccaaaaacaagtattctttacagatacaacatggcgtgatgctcaccaatccttatttgctacacgtttgcgtaccatcgatatggcacgcgtagctggcgatgccgctaaaggtgtaccaaatctattctccttagaatgttggggcggtgcaacatttgacgtatcctatcgcttcttgcacgaagatccatgggaacgtttgcgcatgttccgtaaagaagtgcctaatacattgcttcaaatgttgattcgcggtgctaatgccgttggttatacatcctatcctgataatgtagttcgtaatttcattcaattgtccgctaaaaatggtatcgacgtattccgcgtatttgatagcttgaatagccttgacaatatgaaggttgctattgatgaagttcgcaatcaaaataagattgctgaagttgcattgtgctacacaggcgatatcctcgacagcaatcgtcctaaatacaatcttgattactatgtaaaaatggctaaagaattgcaaaatgctggtgccaatatcattgctattaaagatatggctggtttgttgaaacctcaagctgcatacaacttagtatccgctttgaaagatgctgtaacagtaccaattcatttgcattctcatgaaggttctggcaatactatttattcttatggacgtgctgtagatgctggtgttgacgttattgacttagcatattctgcatttgctaatggcactagccaaccaagcatgaattctatgtattacgctttagctggtactgaacgtcaaccacaaatgaatattgactacatggaagaaatgtctcattacttcggtagtattcgtccttactacagaggcgttgataaagctgaaaaatatccaaatacagaagtataccaacatgaaatgccaggcggtcaatattccaacttgcaacaacaagctaaaatggttggacttggtgatcgttggacagacattaaaaaagtatatcaccaagttaatatgatgtttggtgatatcatcaaggtaacaccttcttctaaagtcgttggtgatatgacattgtacatggtacaaaataatttgactgaaaaagatatctatgaaaaaggtgatactcttgatttccctcaatcagtagtagaattcttcgaaggtcgtctaggtacaccatatcaagggttccctgaagaacttcaaaaaatcatcttgaaaggtgctcgccctattactgttcgtcctggtgctgtattacctccaactgatttcgaacatgttcgtaatgaattaaacgaaatgggtgctaatactactgatgaagatgtaagtgcatactgcttgtatcctaaagtatttaaagactacactaaatttactaaagactttggtaatgtatctgtactagatacaccaacattcttctttggtatgaaacgtggtgaagaaattcaagttactattgaaaaaggtaaaacattaattatcaagatgaatggtgtatccgatcctgatgaagatggtaatcgcatcgttctctttgaattcaatggccaaccacgttctattaaagttcatgataaacatgctaaaacaactggcgttgttcgtcgtaaagtaaatgaatccaaccctggtgaaattggtgctacgttgtctggctccgttgttaagattctagttaagaaaggtcaatccgtaactaaaggtgaaccattaatcgtaacagaagctatgaagatggaaacaacaattacagctcctatcggtggtatcgttgaagaaattcttgttcgtgaaggcagccgtatcgaatccggtgattgcttgttacacattgaagatgctattaaacgttaa

SEQ ID NO: 16 - protein, 451 aa, Veillonella atypica
METRIEYDSMGPVEVDARRIYGPQTQRSFNNFKIGDHRIPIEQIKALALVKKACALTNAKCGAVTEEKAKLIAQVVDEIVDGKWDEEFPLTVFQTGSGTQTNMNVNEVIAHRAKQLDEANPLHPNDDVNRGQSTNDTFPTAMHICGYFEITKRVIPALDGLIVSFEKLQEKGIGLQKVGRTHLQDATFIMVDQEISAFVDGLKTAKTMLLQNADYLLDVALGGTAVGTGVNTPKGYLDVMETVLPEVTGAPFRVKNNKFQGLSLKDAFMMAHGALNTLATTLFKIANDVRFLGSGPRCGYGEWHLPENEPGSSIMPGKVNPTQCEALAMVCAQVFGINTTMTLCAGSGAFQLNVYMPIMIYDFVESCRLLADAMNSFTTHCIDGVEFVPEKLKFFVEQSLMIATSLTPYIGYDKSAKVVKEAYKRGCSIKEIILEEKLMTEDEFAEAVRMK

SEQ ID NO: 17 - protein, 280 aa, Veillonella atypica
MREIQVSEITKTVRQMCMDAAYHLPKDIYEGLKKGRETEESPVGCIVLDQIIKNAEIADAEDRPYCQDTGMTMVFLKVGQDVHFVGGDLTEAINAGVAAGYVEGYLRKSVVAEPLFNRKNTQNNTPAIIYTEIVPGDKVDIQVELKGFGSENKSDVAMLVPADGVEGVKNAVLEIVKHAGPNPCPPIVLGIGIGGTMDQAAVMSKKALLRDISVPHKDADYAKLEEEIMEMVNKTGIGPQLGGTTTCIGVNIEWGATHIAGLPVAVTIMCHAARHAHVVL

SEQ ID NO: 18 - protein, 320 aa, Veillonella atypica
MRQITYHIHRYQQGRAFVQTFKFDYEADRTILWGLQKIKDTQDPTLTFLAACRSAVCGACSIRVNGEAMLGCESKIDELTERYGTDELTIAPIGNFRVIRDLVVDWESKVDRLKTVAPWIFLKAEFNEGDKIVRQTPADFKKFVAGTECILCGCCASECNKLTARQDDFLEPYVFTKANRFVLDSRDDAPMAHIQPAYDNGLWKCVHCMNCISRCPKHLKPAQDISNMRKEATKAGLTNNKGVRHAVAFKEDLYKTGRLKEVSMSLKSDGVVDSAKQAFYALRLWKHSKINPFELVVPQKPVNGIDGVRRLMKAAEEVSK

SEQ ID NO: 19 - protein, 315 aa, Veillonella atypica
MKLRKVGIIGTGHVGSHVAFSLALQGEVDELYMMDIDEKKAKAQAMDVNDAVSYIPHRVTATSGPIEDCGDCDILVFSAGPLPNLYQDRLESLGDTIAVLKDVIPRIKASGFKGFIISISNPADVVATYLCKHLDWNPKRIISSGTALDSARLQKELAHIFDISNRTITAYCMGEHGASAMVPWSHVYVQGKPLVELQKELPHRFPELDHKQVLDDVKIGGYHVLAGKGSTEFGIASATTELIRSVFHDEKKVLPCSCYLDGQYGETGVFASTPAVIGKDGIEDVLELQMTEDELALFKKSCAVIREYAKKAETM

SEQ ID NO: 20 - protein, 509 aa, Veillonella atypica
MQTVQEKIELLHEKLAKVKAGGGEKRVEKQHSQGKMTARERLAKLFDDNSFVELDQFVKHRCVNFGQDKKELPGEGVVTGYGTIDGRLVYAFAQDFTVEGGSLGEMHAAKIVKVQRLAMKMGAPIVGINDSGGARIQEAVDALAGYGKIFFENTNASGVIPQISVIMGPCAGGAVYSPALTDFIYMVKNTSQMFITGPAVIKSVTGEEVTAEDLGGAMAHNSVSGVAHFAAENEDDCIAQIRYLLGFLPSNNMEDAPLVDTGDDPTREDEGLNSLLPDNSNMPYDMKDVIAATVDNGEYYEVQPFYATNIITCFARFDGQSVGIIANQPKVMAGCLDINASDKSSRFIRFCDAFNIPIVNFVDVPGFLPGTNQEWGGIIRHGAKMLYAYSEATVPKITVITRKAYGGSYLAMCSQDLGADQVYAWPTSEIAVMGPAGAANIIFKKDEDKDAKTAKYVEEFATPYKAAERGFVDVVIEPKQTRPAVINALAMLASKRENRAPKKHGNIPL

SEQ ID NO: 21 - protein, 140 aa, Veillonella atypica
MAFKVLQVDHIGIGVNDLAATKEFYKNALGIQHLPEDEVVEEQKVKVSFFPCGDAELEFLETTTPDGPIGKFIEKNGGRDGIQHVALRVDNIENAIADLMAKGIRMIDEKPRYGAGGSSIAFVHPKATGGVLLELCQRMK

SEQ ID NO: 22 - protein, 729 aa, Veillonella atypica
MFKNPDFSSLGLGSQSATSRDAWLAELKKETGKSFEDLYNTTMEQIQLKPLYTEMDYEGMTHLDYMAGVPPFLRGPYSTMYVTRPWTVRQYAGFSTAEESNAFYRRNLAAGQKGLSIAFDLATHRGYDSDHPRVVGDVGKAGVAVDSILDMEILFSGIPLDQMSVSMTMNGAVLPVMAFYILAGEEQGVDKKVMAGTIQNDILKEFMVRNTYIYPPATSMRIIGDIFAYTSQNMPKFNSISISGYHMQEAGATADIELGYTLADGLEYIRTGVNAGLHVDQFAPRLSFFWAIGKNYFMEVAKMRAARMLWAKIIKSFGSENPKSMALRTHSQTSGWSLTEQDPFNNVARTCMEAMGAALGHTQSLHTNALDEAIALPTDFSARIARNTQLYIQDETKVCKVIDPWGGSYYVEALTDELIRRAWGHIQEIESLGGMAKAIDTGLPKMRIEEAAARRQARIDSGREAIVGINKYRLDKEDPLDILDVDNTAVREAQIRRLEQLRANRDEDKVQSCLEAITNATESGEGNLLALALEAARARASLGEISFAVEKVCGRHKAVIRSISGVYSSEYEDDDVIKEVRQMADDFEELEGRRPRIMIAKMGQDGHDRGAKVIATSFADMGFDVDIGPLFQTPEETAQDAVDNDVHIVGFSSLAAGHKTLLPQLVEELNKRGRGDILVAIGGVIPAQDYEFLREHGAVAIFGPGTVLPVAAKKLLETLTSHVQDEGND

SEQ ID NO: 23 - protein, 1148 aa, Veillonella atypica
MKKIKSVLVANRGEIAIRVFRACNEMGIKTVAIYSKEDTLSLHRNQADEAYLVGEGKKPVDAYLDIEDIIRIAKEHDIDAIHPGYGFLSENEEFARRCGEEGIIFIGPHVEHLNMFGDKVNARTQAKLADIPMIPGSDGALRDFAQLEEFAETHGYPLMIKAVNGGGGRGMREVHRKEDLRDAYDRAKSEAKAAFGDDDVYVEKLIVEPKHIEVQILGDEHGNVVHLHERDCSVQRRHQKVVEMAPAFALPLETRKAVCDAAVKIMKNVGYVNAGTVEFLVTSDGSFYFIEVNPRIQVEHTVTEMITDIDIVHSQIRIAEGYDLHSPEIGIPEQDEIPCKGTAIQCRITTEDPKNNFMPDTGKILAYRSSGGFGIRLDSGNAFTGAVVTPYYDSLLVKATAFGPNNEETIRKMLRCLKEFRIRGVKTNIHFLINVLENPEFQSGNYTVNFIEDHPELFELKPDRDRGTKLLRYIADTTINGYSGAGPQEVPDFEPMQLPSKLDVSPAPGTKQKFDELGPEGFSKWLADQKQVFFTDTTWRDAHQSLFATRLRTIDMARVAGDAAKGVPNLFSLECWGGATFDVSYRFLHEDPWERLRMFRKEVPNTLLQMLIRGANAVGYTSYPDNVVRNFIQLSAKNGIDVFRVFDSLNSLDNMKVAIDEVRNQNKIAEVALCYTGDILDSNRPKYNLDYYVKMAKELQNAGANIIAIKDMAGLLKPQAAYNLVSALKDAVTVPIHLHSHEGSGNTIYSYGRAVDAGVDVIDLAYSAFANGTSQPSMNSMYYALAGTERQPQMNIDYMEEMSHYFGSIRPYYRGVDKAEKYPNTEVYQHEMPGGQYSNLQQQAKMVGLGDRWTDIKKVYHQVNMMFGDIIKVTPSSKVVGDMTLYMVQNNLTEKDIYEKGDTLDFPQSVVEFFEGRLGTPYQGFPEELQKIILKGARPITVRPGAVLPPTDFEHVRNELNEMGANTTDEDVSAYCLYPKVFKDYTKFTKDFGNVSVLDTPTFFFGMKRGEEIQVTIEKGKTLIIKMNGVSDPDEDGNRIVLFEFNGQPRSIKVHDKHAKTTGVVRRKVNESNPGEIGATLSGSVVKILVKKGQSVTKGEPLIVTEAMKMETTITAPIGGIVEEILVREGSRIESGDCLLHIEDAIKR

(19) United States
(12) Patent Application Publication
(10) Pub. No.: US 2022/0054559 A1
(43) Pub. Date: Feb. 24, 2022

(54) COMPOSITIONS AND METHODS FOR ENHANCING EXERCISE ENDURANCE

(71) Applicants: PRESIDENT AND FELLOWS OF HARVARD COLLEGE, Cambridge, MA (US); JOSLIN DIABETES CENTER, INC., Boston, MA (US)

(72) Inventors: Jonathan SCHEIMAN, Cambridge, MA (US); George M. CHURCH, Cambridge, MA (US); Aleksandar D. KOSTIC, Cambridge, MA (US); Theodore Andrew CHAVKIN, Cambridge, MA (US); Jacob Mayne LUBER, Cambridge, MA (US)

(73) Assignees: PRESIDENT AND FELLOWS OF HARVARD COLLEGE, Cambridge, MA (US); JOSLIN DIABETES CENTER, INC., Boston, MA (US)

(21) Appl. No.: 17/432,647

(22) PCT Filed: Feb. 21, 2020
(86) PCT No.: PCT/US2020/019328

§ 371 Date: Aug. 20, 2021

Related U.S. Application Data
(60) Provisional application No. 62/808,464, filed on Feb. 21, 2019.

(51) Int. Cl. A61K 35/74; A23L 33/135; C12N 1/20

(52) U.S. Cl. CPC A61K 35/74; C12N 1/205; A23L 33/135

(57) ABSTRACT

The technology described herein is directed to compositions and methods for enhancing exercise endurance. In some embodiments of any of the aspects, a composition comprises a Veillonella probiotic bacteria. In some embodiments of any of the aspects, Veillonella probiotic bacteria and or propionate is administered to a patient in need thereof. In some embodiments of any of the aspects, a composition comprises a probiotic bacteria engineered to express genes encoding enzymes sufficient to metabolize the conversion of lactate into short-chain fatty acids comprising one or more of propionate and acetate.

### CROSS-REFERENCE TO RELATED APPLICATIONS

This application claims benefit under 35 U.S.C. § 119(e) of U.S. Provisional Application No. 62/808,464 filed Feb. 21, 2020, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Feb. 21, 2020, is named 002806-094390WOPT_SL.txt and is 57,671 bytes in size.

### TECHNICAL FIELD

The technology described herein relates to compositions and methods for enhancing exercise endurance.

### BACKGROUND

Human microbiome studies to-date have generally focused on the “healthy” individual in aggregate or on individuals with disease, identifying features of the microbiome correlated with and even causally involved in the maintenance of health or promotion of disease. Studying the microbiome of extraordinarily healthy individuals may offer new insights into possible roles for the microbiome in achieving or maintaining health.

Previous studies have found athlete microbiomes to contain distinct microbial compositions defined by elevated abundances of Veillonella, Bacteroides, Prevotella, Methanobrevibacter or the Akkermansiaceae. These studies show that exercise alters the composition of the gut microbiome. However, it remains unknown what are the downstream effects of these alterations, or whether these alterations may feed back to affect exercise phenotypes.

The gut microbiome is a powerful metabolic engine that can have broad impacts on host metabolism. For example, gut microbiota transfer from obese mice to germ-free animals results in significantly increased body weight and adiposity relative to mice that received the transfer from a lean donor, and this observation extends to human donors. Indeed, fecal microbiota transplant from healthy, lean human donors results in significant improvements to insulin sensitivity in human recipients with insulin resistance. The gut microbiome metabolizes dietary phosphatidylcholine into trimethylamine, which is processed in the liver into trimethylamine N-oxide (TMAO). Microbiota depletion by antibiotics negates choline-induced exacerbation of atherosclerosis in a mouse model, and plasma levels of TMAO and its precursors choline and betaine predict the risk of cardiovascular disease in humans. Phenylketonuria is a genetic disease characterized by the inability to metabolize phenylalanine (Phe), leading to neurotoxicity. It was recently demonstrated that colonization with an E. coli engineered to express Phe-metabolizing genes significantly reduced blood Phe concentrations in mouse and non-human primate models, thus serving as a new class of therapeutic drug candidates to treat human metabolic diseases.

### SUMMARY

The technology described herein is directed to compositions and methods of enhancing exercise endurance. Described herein are the results of studies on the gut microbiome of exceptionally healthy individuals, in particular, endurance sport athletes. The results indicate that the gut microbiota of endurance athletes tend to be enriched in the abundance of Veillonella bacterial species. It is demonstrated herein that such Veillonella species, administered to mice, can improve their exercise endurance. It is also demonstrated herein that administering the lactate metabolite propionate can increase exercise endurance in the mouse model. In various embodiments, compositions described comprise Veillonella bacterial species, fractions thereof, and/or metabolites they produce, formulated for administration to a subject who desires or needs increased exercise endurance. In other embodiments, methods comprise the administration of such compositions or formulations to a subject who desires or needs increased exercise endurance.

In one aspect, described herein is a composition comprising at least one Veillonella probiotic bacterium and an acceptable excipient, diluent, or carrier.

In some embodiments of this and other aspects described herein, the Veillonella probiotic bacterium is selected from the group consisting of Veillonella atypica, Veillonella dispar, and Veillonella parvula.

In some embodiments of this and other aspects described herein, the Veillonella probiotic bacterium comprises Veillonella atypica, Veillonella dispar, and Veillonella parvula.

In some embodiments of this and other aspects described herein, the Veillonella probiotic bacterium comprises and expresses genes encoding enzymes sufficient to metabolize the conversion of lactate into short-chain fatty acids comprising one or more of propionate and acetate.

In some embodiments of this and other aspects described herein, the Veillonella probiotic bacterium comprises and expresses genes encoding enzymes from the methylmalonyl-CoA pathway.

In some embodiments of this and other aspects described herein, the Veillonella probiotic bacterium is viable and lyophilized.

In some embodiments of this and other aspects described herein, the acceptable excipient, diluent, or carrier comprises water, saline, dextrose, glycerol, ethanol or the like and combinations thereof.

In some embodiments of this and other aspects described herein, the composition is in the form of a pill, a tablet, or a capsule.

In another aspect, described herein is a method of enhancing exercise endurance, comprising administering an effective dose of any composition described herein to a subject in need thereof.

In some embodiments of this and other aspects described herein, the composition is administered via an oral, enteric, gastrointestinal, rectal, or parenteral route.

In some embodiments of this and other aspects described herein, the subject is a human athlete or human in need of enhanced exercise endurance.

In some embodiments of this and other aspects described herein, enhanced exercise endurance comprises increased time spent on an exercise until exhaustion time.

In another aspect, described herein is a device preloaded for administration to a body cavity comprising an effective dose of propionate to enhance exercise endurance.

In some embodiments of this and other aspects described herein, the propionate comprises 10 mM-1000 mM sodium propionate.

In some embodiments of this and other aspects described herein, the device comprises a suppository.

In some embodiments of this and other aspects described herein, the body cavity is the rectum.

In another aspect, described herein is a method of enhancing exercise endurance comprising administering to a subject in need thereof an effective amount of propionate.

In some embodiments of this and other aspects described herein, the propionate comprises 10 mM-1000 mM sodium propionate.

In some embodiments of this and other aspects described herein, the propionate is administered via a rectal, intracolonic, gastrointestinal, enteric, oral, or parenteral route.

In some embodiments of this and other aspects described herein, the subject is a human athlete or human in need of enhanced exercise endurance.

In another aspect, described herein is an engineered probiotic bacterium that is effective at enhancing exercise endurance.

In some embodiments of this and other aspects described herein, the engineered probiotic bacterium comprises and expresses genes encoding enzymes sufficient to metabolize the conversion of lactate into short-chain fatty acids comprising one or more of propionate and acetate.

In some embodiments of this and other aspects described herein, the engineered probiotic bacterium comprises and expresses genes encoding enzymes from the methylmalonyl-CoA pathway.

In some embodiments of this and other aspects described herein, the engineered probiotic bacterium comprises and expresses genes encoding enzymes selected from the group consisting of: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.

In some embodiments of this and other aspects described herein, enhanced exercise endurance comprises increased time spent on an exercise until exhaustion time.

In another aspect, described herein is a food, beverage, or dietary supplement composition comprising a bacterium that comprises and expresses genes encoding enzymes sufficient to metabolize the conversion of lactate into short-chain fatty acids comprising one or more of propionate and acetate.

In some embodiments of this and other aspects described herein, the bacterium is present in an amount sufficient to increase exercise endurance in a subject consuming the subject.

In some embodiments of this and other aspects described herein, the bacterium is selected from the group consisting of Veillonella atypica, Veillonella dispar, and Veillonella parvula.

In some embodiments of this and other aspects described herein, the bacterium comprises and expresses genes encoding enzymes from the methylmalonyl-CoA pathway.

In some embodiments of this and other aspects described herein, the bacterium comprises and expresses genes encoding enzymes selected from the group consisting of: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.

In another aspect, described herein is a composition as described herein, for use in enhancing exercise endurance.

In another aspect, described herein is a device as described herein, for use in enhancing exercise endurance.

In another aspect, described herein is an engineered probiotic bacterium as described herein, for use in enhancing exercise endurance.

In another aspect, described herein is a food, beverage, or dietary supplement composition as described herein, for use in enhancing exercise endurance.

In another aspect, described herein is use of a composition as described herein, for enhancing exercise endurance.

In another aspect, described herein is use of a device as described herein, for enhancing exercise endurance.

In another aspect, described herein is use of an engineered probiotic bacterium as described herein, for enhancing exercise endurance.

In another aspect, described herein is use of a food, beverage, or dietary supplement composition as described herein, for enhancing exercise endurance.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-FIG. 1D is a series of graphs showing the longitudinal composition of the marathon runner microbiome. FIG. 1A: Phylum level relative abundance partitioned by individual and time (−5 to +5 days in relation to running the Marathon) shows few global differences in composition. FIG. 1B: Veillonella relative abundance at the Genus level partitioned by individual and time (−5 to +5 days in relation to running the Marathon) shows that Veillonella has a significant difference in relative abundance (P=0.02; Wilcoxon rank sum test with continuity correction) between samples collected before and after exercise. FIG. 1C: Generalized linear mixed effect models (GLMMs) predicting longitudinal Veillonella relative abundance in the marathon participants. Differences in intercept between fits for different marathoners represent random effects. FIG. 1D: 95% confidence intervals for all fixed effects (coefficients) included in the GLMMs. The Y-axis represents Veillonella relative abundance and the X-axis represents (time days in relation to running the Marathon). All coefficients except time (P=0.0014, Wald Z-test, post-marathon time points correspond with increased Veillonella relative abundance) are not significant, suggesting that Veillonella blooms in runners correspond with exercise state and not other fixed effects.

FIG. 2A-FIG. 2B is a series of graphs showing that Veillonella gavage improves treadmill runtime in mice. FIG. 2A: Mice gavaged with Veillonella atypica have greater maximum run time per week than mice gavaged with Lactobacillus bulgaricus in an AB/BA crossover trial (n=32). Data shown are the maximum run time out of 3 days of consecutive treadmill running for a given treatment (all mice switched treatments second week). The jitter plot shows each mouse as an individual point, with the central bar representing the mean and error bars representing standard error of the mean (SEM). (n=32). (*P<0.05, using paired t-test). FIG. 2B: Generalized linear mixed effect models (GLMMs) predicting runtime in the 2 week AB/BA crossover trial. The Y-axis shows seconds run on treadmill until exhaustion, and the X-axis shows days which the mice were run in the 2 week crossover. Color of lines (GLMM fits) and points (runs by an arbitrary mouse) represents treatment sequence; shape of points represents treatment at a given time point. These models incorporate both random effects (individual variation per mouse that manifests longitudinally) and fixed effects (treatment day, treatment sequence, and treatment given). Visualization of all longitudinal data points with the GLMM predictions overlaid show both the effect of Veillonella atypica increasing performance on both sides of the crossover when aggregated by treatment group (thick lines) as well as the trends for each of the 32 individual mice (thin lines). (*P<0.05, Wald-Z test on model coefficients).

FIG. 3A-FIG. 3C is a series of schematics and heat maps showing that global athlete stool metagenomics show enrichment for the Veillonella methylmalonyl-CoA pathway that converts lactate to the short chain fatty acid (SCFA) propionate. FIG. 3A: The methylmalonyl-CoA pathway and inset showing significant differentially expressed gene families pathway-wide in a pair of non-redundant gene catalogs created from metagenomic sequencing of athlete stool samples. Log transformed relative abundance increases after exercise for every enzyme in the methylmalonyl-CoA pathway. (**P<0.01; Fisher's exact test). FIG. 3B: Bacterial phylogenetic tree showing diversity of microbes that have the ability to utilize lactate as a carbon source. FIG. 3C: Prevalence of enzymes in the methylmalonyl-CoA pathway that breaks down lactate into acetate and propionate in reference genomes from the representative subset of lactate processing microbes.

FIG. 4A-Fig. 4C: A series of schematics and bar graphs showing that isotope-labeled lactate is detected in the intestinal lumen when injected intravenously. FIG. 4A: Schematic of the experimental design. Mice were injected with ¹³C₃ sodium lactate, then sacrificed after 12 minutes. Serum and plasma were collected via cardiac puncture. Cecum and colon contents were collected by dissection. FIG. 4B: Abundance of ¹³C₃ lactate quantified relative to abundance of unlabeled lactate. FIG. 4C: ¹³C₃ lactate abundance normalized to the expected natural abundance of ¹³C₃ lactate. Ratio of labeled/unlabeled lactate was quantified for experimental samples as well as for unlabeled lactate standard. Experimental samples are represented as fold-change relative to unlabeled standard. Data are means ±SEM, (n=7). (**P<0.01; ***P<0.001; using one sample t-test with FDR correction).

FIG. 5: Intracolonic infusion of propionate improves maximum run time in mice. Data shown are the maximum run time out of 3 days of consecutive treadmill running The jitter plot shows each mouse as an individual point, with the central bar representing the mean and error bars representing s.e.m. (n=8). (*P<0.05, using Welch's t-test).

FIG. 6: A schematic showing the proposed model of the microbiome-exercise interaction. Black arrows represent the well known steps of the Cori cycle, where glucose is converted to lactate in the muscle, enters the liver via blood circulation, then is converted back to glucose in the liver via gluconeogenesis. Red arrows represent the steps outlined herein. Without wishing to be bound by theory, it is proposed that first, lactate produced in the muscle enters the intestinal lumen via blood circulation. In the intestine it acts as a carbon source for specific microbes, including Veillonella species. This causes the observed bloom in intestinal Veillonella, as well as production of SCFA byproducts (predominantly propionate), which are taken up by the host via the intestinal epithelium. Presence of microbiome-sourced SCFAs in the blood improves athletic performance. Together, this creates an addendum to the Cori cycle by converting an exercise byproduct into a performance-enhancing molecule, mediated by naturally occurring members of the athlete gut microbiome.

FIG. 7: An image showing the coefficients and correlations from 16S GLMM analysis.

FIG. 8: Histogram of p-values for time coefficient from LOOCV models predicting 16S Veillonella abundance. Red line represents p value for model trained without any hold outs.

FIG. 9: Histogram of p-values for time coefficient from 1000 label permutations in GLMM models predicting Veillonella relative abundance. Red line represents p value for model trained without any label permutation.

FIG. 10A-FIG. 10B: A series of graphs showing control subjects. FIG. 10A: 16S composition in control subjects. FIG. 10B: Veillonella relative abundance in control subjects.

FIG. 11: Density plot of max run times in AB/BA crossover study. Shapiro-Wilk's normality test on the max run times for each mouse in each treatment group results in p=0.6703 with a null hypothesis that the distribution of the data is normal, n=64.

FIG. 12: A series of box plots showing 95% confidence intervals for coefficient effect on treadmill runtime in AB/BA crossover.

FIG. 13: An image showing coefficients and correlations from AB/BA crossover study GLMM analysis.

FIG. 14: Histogram of p-values for treatment coefficient from LOOCV models predicting treadmill runtime. Red line represents p value for model trained without any hold outs.

FIG. 15: Histogram of p-values for treatment coefficient from 1000 label permutations in GLMM models predicting treadmill runtime. Red line represents p value for model trained without any label permutation.

FIG. 16: A dot plot showing AB/BA crossover study results segregated by individual mouse. Each of the 32 facets (each representing an individual mouse) has 6 longitudinal treadmill run times plotted (3 pre and 3 post treatment crossover). Shape of points represent treatment sequence. Each mouse facet has two horizontal lines showing mean runtime when dosed Lb. bulgaricus (light blue) and when dosed V. atypica (light red). Each facet has a GLMM fit to all data in a treatment sequence (green), a LOOCV GLMM fit trained on all mice except for the mouse the facet represents (red) and a GLMM fit showing change in intercept related to random effect for each mouse (blue).

FIG. 17: A bar graph showing the difference in maximum run time between V. atypica gavage periods and Lb. Bulgaricus gavage treatment periods segregated into “responders” and “non-responders” to V. atypica treatment.

FIG. 18A-FIG. 18B: A set of bar graphs showing cytokines after V. atypica (green) and L. bulgaricus (blue) gavage or baseline (blue). FIG. 18A: Serum cytokine levels of mIL-6. FIG. 18B: Serum cytokine levels of mIFNγ, mIL10, mIL12p70, mIL17, mIL1β, mIL2, and mTNFα.

FIG. 19A-FIG. 19B: A series of blots and dot plots showing GLUT4 abundance. FIG. 19A: GLUT4 abundance in pre-exercise states as well as following Lb. bulgaricus and V. atypica gavage. FIG. 19B: Fold-change in GLUT4 abundance.

FIG. 20A-FIG. 20C: A series of bar graphs and heat maps showing shotgun metagenomic sequencing performed on stool samples (n=87) from ultra-marathoners and rowers both before and after exercise. FIG. 20A: Fraction of putative Veillonella relative abundance from metagenomics (calculated utilizing metaphlan2) before and after exercise in rowers and runners. FIG. 20B: Significant alleles (calculated from pairwise ANOVA) that are present in each of the 87 samples. FIG. 20C: 396 significant alleles from FIG. 20B segregated by exercise state and sample.

FIG. 21: Histogram comparing non-redundant gene family size and annotation fraction.

FIG. 22: A schematic showing enzyme resolution log transformed relative abundances of differentially abundant non-redundant gene families mapped by Enzyme Commission (EC) ID to Methylmalonyl-CoA pathway components.

FIG. 23A-FIG. 23B: A line graph and dot plot showing lactate clearance following IP injection in mice. FIG. 23A: Mice were gavaged with either Veillonella atypica or Lactobacillus bulgaricus and 5 hours later injected with sodium lactate (750 mg/kg). 5 minutes post-injection and every 10 minutes after that, blood lactate was measured (n=16). FIG. 23B: Area under the curve (AUC) was determined for each mouse and compared between treatments. Statistical analysis was done using an unpaired two-tailed t-test.

FIG. 24A-FIG. 24B: A set of bar graphs showing cytokines after intra-rectal propionate instillation (grey), PBS (blue), or baseline (green). FIG. 24A: Cytokine levels of mIFNγ, mIL10, mIL12p70, mIL17, mIL1β, mIL2, and mTNFα. FIG. 24B: Cytokine levels of mIL-6.

### DETAILED DESCRIPTION

Described herein are the results of studies on the gut microbiome of exceptionally healthy individuals, in particular, endurance sport athletes. The results indicate that the gut microbiota of endurance athletes tend to be enriched in the abundance of Veillonella bacterial species. It is demonstrated herein that such Veillonella species, administered to mice, can improve their exercise endurance. It is also demonstrated herein that administering the lactate metabolite propionate can increase exercise endurance in the mouse model. In view of this, described herein are methods and compositions for increasing exercise endurance. In various embodiments, compositions described comprise Veillonella bacterial species, fractions thereof, and/or metabolites they produce, formulated for administration to a subject who desires or needs increased exercise endurance. In other embodiments, methods comprise the administration of such compositions or formulations to a subject who desires or needs increased exercise endurance. The following discusses considerations involved in the preparation and use of compositions described and their use in the methods described.

### Probiotic Bacteria

As described herein, some embodiments comprise at least one Veillonella probiotic bacterium. The genus Veillonella belongs to the family Veillonellaceae, which is comprised of gram-negative anaerobic cocci. The genus Veillonella is subdivided into 13 species, at least 7 of which have been isolated in humans. These species include V. parvula, V. atypica, V. dispar, V. denticariosi, V. rogosae, V. tobetsuensis, and V. montpellierensis. In some embodiments described herein, the Veillonella probiotic bacterium can be Veillonella atypica, Veillonella dispar, and or Veillonella parvula.

Veillonella are commonly found in the human intestine as well as in the guts of other mammals. The bacteria of this genus are known for their lactate-fermenting abilities. Lactate is a product of lactic acid, a product of cell metabolism that can accumulate when cells lack sufficient oxygen. Although Veillonella themselves are considered largely non-pathogenic (i.e. they do not cause disease), elevated levels have been observed in patients suffering from infections associated with conditions such as immunodeficiency.

Laboratory identification of Veillonella bacteria is well known in the art. The genus Veillonella produces small, round colonies with raised centers ranging from 0.5 mm to 1.0 mm in diameter. The colonies have a gray-green appearance on blood-containing media. The colonies fail to grow in air or 5% CO₂ in air. Gram's stain reveals very small gram-negative cocci in clumps, pairs or short chains. Veillonella sp. are nonfermentative and produce acetic and propionic acids. They are catalase variable. Identification is aided by the organsisms' ability to reduce nitrate to nitrite, which is determined by performing with a disc test. Confirmation of the genus Veillonella requires gas-liquid chromatography. Speciation requires restriction fragment length polymorphism analysis of PCR-amplified 16S ribosomal DNA.

In some embodiments, the Veillonella probiotic bacterium can be isolated and cultured from a subject or specimen (e.g., human athlete, mouse). As described herein, a composition can comprise an engineered probiotic bacterium. The engineered probiotic bacterium can be a Veillonella species or any probiotic bacteria species that can be engineered to express enzymes from the methylmalonyl-CoA pathway. Probiotic bacteria species that can be engineered are well known in the art and comprise but are not limited to Bacteroides, Prevotella, Methanobrevibacter, Akkermansiaceae, Lactobacillus, or Bifidobacteriaceae species.

In some embodiments of any of the aspects, the engineered probiotic bacterium comprises and expresses genes encoding enzymes sufficient to metabolize the conversion of lactate into short-chain fatty acids including one or more of propionate and acetate. In some embodiments of any of the aspects, the engineered probiotic bacterium comprises and expresses genes encoding enzymes from the methylmalonyl-CoA pathway. In some embodiments of any of the aspects, enzymes from the methylmalonyl-CoA pathway comprise Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.

Nucleic acids and methods used to engineer (e.g., transform exogenous nucleic acids into) Veillonella are well known in the art (see e.g., Liu et al. 2012, FEMS Microbiol Lett 322(2): 138-144; Liu et al. 2012 Appl Environ Microbiol. 78(9): 3488-3491; Knapp et al. 2017, Front. Cell. Infect. Microbiol 7: 139; all of which are incorporated herein by reference in their entireties). Non-limiting examples of nucleic acids suitable to transform Veillonella include genomic DNA, a PCR product, or a plasmid. Non-limiting examples of suitable plasmids or vectors include: pVJLl or pBSIL1. Veillonella bacteria can be transformed using methods comprising but not limited to electroporation or natural competence. Nucleic acids used to transform Veillonella can comprise genes encoding enzymes from the methylmalonyl-CoA pathway, as described below. Nucleic acids and methods used to engineer (e.g., transform exogenous nucleic acids into) probiotic bacteria such as Lactobacillus well known in the art (see e.g., US 2014/0045235 A1).

In some embodiments of any of the aspects, the probiotic bacteria comprises a 16S rRNA sequence as described herein. In some embodiments of any of the aspects, the nucleic acid sequence of the probiotic bacterial 16S rRNA sequence comprises SEQ ID NO: 5, 6, or 7 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 5-7.

SEQ ID NO: 5, V. atypica 16S rRNA, 1569 nucleotides (nt)
TTGGAGAGTTTGATCCTGGCTCAGGACGAACGCTGGCGGCGTGCTTAACACATGCAAGTC
GAACGAAGAGCGATGGAAGCTTGCTTCTATCAATCTTAGTGGCGAACGGGTGAGTAACG
CGTAATCAACCTGCCCTTCAGAGGGGGACAACAGTTGGAAACGACTGCTAATACCGCAT
ACGATCCAATCTCGGCATCGAGACTGGATGAAAGGTGGCCTCTATTTATAAGCTATCACT
GAAGGAGGGGATTGCGTCTGATTAGCTAGTTGGAGGGGTAACGGCCCACCAAGGCGATG
ATCAGTAGCCGGTCTGAGAGGATGAACGGCCACATTGGGACTGAGACACGGCCCAGACT
CCTACGGGAGGCAGCAGTGGGGAATCTTCCGCAATGGACGAAAGTCTGACGGAGCAACG
CCGCGTGAGTGATGACGGCCTTCGGGTTGTAAAGCTCTGTTAATCGGGACGAATGGTTCT
TGTGCGAATAGTGCGAGGATTTGACGGTACCGGAATAGAAAGCCACGGCTAACTACGTG
CCAGCAGCCGCGGTAATACGTAGGTGGCAAGCGTTGTCCGGAATTATTGGGCGTAAAGC
GCGCGCAGGCGGATCAGTTAGTCTGTCTTAAAAGTTCGGGGCTTAACCCCGTGATGGGAT
GGAAACTGCTGATCTAGAGTATCGGAGAGGAAAGTGGAATTCCTAGTGTAGCGGTGAAA
TGCGTAGATATTAGGAAGAACACCAGTGGCGAAGGCGACTTTCTGGACGAAAACTGACG
CTGAGGCGCGAAAGCCAGGGGAGCGAACGGGATTAGATACCCCGGTAGTCCTGGCCGTA
AACGATGGGTACTAGGTGTAGGAGGTATCGACCCCTTCTGTGCCGGAGTTAACGCAATA
AGTACCCCGCCTGGGGAGTACGACCGCAAGGTTGAAACTCAAAGGAATTGACGGGGGCC
CGCACAAGCGGTGGAGTATGTGGTTTAATTCGACGCAACGCGAAGAACCTTACCAGGTC
TTGACATTGATGGACAGAACCAGAGATGGTTCCTCTTCTTCGGAAGCCAGAAAACAGGT
GGTGCACGGTTGTCGTCAGCTCGTGTCGTGAGATGTTGGGTTAAGTCCCGCAACGAGCGC
AACCCCTATCTTATGTTGCCAGCACTTCGGGTGGGAACTCATGAGAGACTGCCGCAGACA
ATGCGGAGGAAGGCGGGGATGACGTCAAATCATCATGCCCCTTATGACCTGGGCTACAC
ACGTACTACAATGGGAGTTAATAGACGGAAGCGAAACCGCGAGGTGGAGCAAACCCGA
GAAACACTCTCTCAGTTCGGATCGTAGGCTGCAACTCGCCTACGTGAAGTCGGAATCGCT
AGTAATCGCAGGTCAGCATACTGCGGTGAATACGTTCCCGGGCCTTGTACACACCGCCCG
TCACACCACGAAAGTCGGAAGTGCCCAAAGCCGGTGGGGTAACCTTCGGGAGCCAGCCG
TCTAAGGTAAAGTCGATGATTGGGGTGAAGTCGTAACAAGGTAGCCGTATCGGAAGGTG
CGGCTGGATCACCTCCTTTCTAGGGAGA
SEQ ID NO: 6, V. dispar 16S rRNA, 1569 nt
TTGGAGAGTTTGATCCTGGCTCAGGACGAACGCTGGCGGCGTGCTTAACACATGCAAGTC
GAACGAAGAGCGATGGAAGCTTGCTTCTATCAATCTTAGTGGCGAACGGGTGAGTAACG
CGTAATCAACCTGCCCTTCAGAGGGGGACAACAGTTGGAAACGACTGCTAATACCGCAT
ACGATCCAATCTCGGCATCGAGGATAGATGAAAGGTGGCCTCTATTTATAAGCTATCACT
GAAGGAGGGGATTGCGTCTGATTAGCTAGTTGGAGGGGTAACGGCCCACCAAGGCGATG
ATCAGTAGCCGGTCTGAGAGGATGAACGGCCACATTGGGACTGAGACACGGCCCAGACT
CCTACGGGAGGCAGCAGTGGGGAATCTTCCGCAATGGACGAAAGTCTGACGGAGCAACG
CCGCGTGAGTGATGACGGCCTTCGGGTTGTAAAGCTCTGTTAATCGGGACGAAAGGCCTT
CTTGCGAATAGTTAGAAGGATTGACGGTACCGGAATAGAAAGCCACGGCTAACTACGTG
CCAGCAGCCGCGGTAATACGTAGGTGGCAAGCGTTGTCCGGAATTATTGGGCGTAAAGC
GCGCGCAGGCGGATTGGTCAGTCTGTCTTAAAAGTTCGGGGCTTAACCCCGTGATGGGAT
GGAAACTGCCAATCTAGAGTATCGGAGAGGAAAGTGGAATTCCTAGTGTAGCGGTGAAA
TGCGTAGATATTAGGAAGAACACCAGTGGCGAAGGCGACTTTCTGGACGAAAACTGACG
CTGAGGCGCGAAAGCCAGGGGAGCGAACGGGATTAGATACCCCGGTAGTCCTGGCCGTA
AACGATGGGTACTAGGTGTAGGAGGTATCGACCCCTTCTGTGCCGGAGTTAACGCAATA
AGTACCCCGCCTGGGGAGTACGACCGCAAGGTTGAAACTCAAAGGAATTGACGGGGGCC
CGCACAAGCGGTGGAGTATGTGGTTTAATTCGACGCAACGCGAAGAACCTTACCAGGTC
TTGACATTGATGGACAGAACTAGAGATAGTTCCTCTTCTTCGGAAGCCAGAAAACAGGT
GGTGCACGGTTGTCGTCAGCTCGTGTCGTGAGATGTTGGGTTAAGTCCCGCAACGAGCGC
AACCCCTATCTTATGTTGCCAGCACTTTGGGTGGGAACTCATGAGAGACTGCCGCAGACA
ATGCGGAGGAAGGCGGGGATGACGTCAAATCATCATGCCCCTTATGACCTGGGCTACAC
ACGTACTACAATGGGAGTTAATAGACGGAAGCAATACCGCGAGGTGGAGCAAACCCGA
GAAACACTCTCTCAGTTCGGATCGTAGGCTGCAACTCGCCTACGTGAAGTCGGAATCGCT
AGTAATCGCAGGTCAGCATACTGCGGTGAATACGTTCCCGGGCCTTGTACACACCGCCCG
TCACACCACGAAAGTCGGAAGTGCCCAAAGCCGGTGGGGTAACCTTCGGGAGCCAGCCG
TCTAAGGTAAAGTCGATGATTGGGGTGAAGTCGTAACAAGGTAGCCGTATCGGAAGGTG
CGGCTGGATCACCTCCTTTCTAGGGAGA
SEQ ID NO: 7, V. parvula 16S rRNA, 1569 nt
TTGGAGAGTTTGATCCTGGCTCAGGACGAACGCTGGCGGCGTGCTTAACACATGCAAGTC
GAACGAAGAGCGATGGAAGCTTGCTTCTATCAATCTTAGTGGCGAACGGGTGAGTAACG
CGTAATCAACCTGCCCTTCAGAGGGGGACAACAGTTGGAAACGACTGCTAATACCGCAT
ACGATCTAACCTCGGCATCGAGGAAAGATGAAAGGTGGCCTCTATTTATAAGCTATCACT
GAAGGAGGGGATTGCGTCTGATTAGCTAGTTGGAGGGGTAACGGCCCACCAAGGCGATG
ATCAGTAGCCGGTCTGAGAGGATGAACGGCCACATTGGGACTGAGACACGGCCCAGACT
CCTACGGGAGGCAGCAGTGGGGAATCTTCCGCAATGGACGAAAGTCTGACGGAGCAACG
CCGCGTGAGTGATGACGGCCTTCGGGTTGTAAAGCTCTGTTAATCGGGACGAAAGGCCTT
CTTGCGAACAGTTAGAAGGATTGACGGTACCGGAATAGAAAGCCACGGCTAACTACGTG
CCAGCAGCCGCGGTAATACGTAGGTGGCAAGCGTTGTCCGGAATTATTGGGCGTAAAGC
GCGCGCAGGCGGATCAGTTAGTCTGTCTTAAAAGTTCGGGGCTTAACCCCGTGATGGGAT
GGAAACTGCTGATCTAGAGTATCGGAGAGGAAAGTGGAATTCCTAGTGTAGCGGTGAAA
TGCGTAGATATTAGGAAGAACACCAGTGGCGAAGGCGACTTTCTGGACGAAAACTGACG
CTGAGGCGCGAAAGCCAGGGGAGCGAACGGGATTAGATACCCCGGTAGTCCTGGCCGTA
AACGATGGGTACTAGGTGTAGGAGGTATCGACCCCTTCTGTGCCGGAGTTAACGCAATA
AGTACCCCGCCTGGGGAGTACGACCGCAAGGTTGAAACTCAAAGGAATTGACGGGGGCC
CGCACAAGCGGTGGAGTATGTGGTTTAATTCGACGCAACGCGAAGAACCTTACCAGGTC
TTGACATTGATGGACAGAACCAGAGATGGTTCCTCTTCTTCGGAAGCCAGAAAACAGGT
GGTGCACGGTTGTCGTCAGCTCGTGTCGTGAGATGTTGGGTTAAGTCCCGCAACGAGCGC
AACCCCTATCTTATGTTGCCAGCACTTTGGGTGGGAACTCATGAGAGACTGCCGCAGACA
ATGCGGAGGAAGGCGGGGATGACGTCAAATCATCATGCCCCTTATGACCTGGGCTACAC
ACGTACTACAATGGGAGTTAATAGACGGAAGCGAGATCGCGAGATGGAGCAAACCCGA
GAAACACTCTCTCAGTTCGGATCGTAGGCTGCAACTCGCCTACGTGAAGTCGGAATCGCT
AGTAATCGCAGGTCAGCATACTGCGGTGAATACGTTCCCGGGCCTTGTACACACCGCCCG
TCACACCACGAAAGTCGGAAGTGCCCAAAGCCGGTGGGGTAACCTTCGGGAGCCAGCCG
TCTAAGGTAAAGTCGATGATTGGGGTGAAGTCGTAACAAGGTAGCCGTATCGGAAGGTG
CGGCTGGATCACCTCCTTTCTAGGGAGA

In some embodiments of any of the aspects, the probiotic bacteria comprises at least a portion of the genome of V. atypica (see e.g., RefSeq: NZ_AMEX00000000.1, genome assembly ID 254577, available on the world wide web at ncbi.nlm.nih.gov/genome/3030?genome_assembly_id=254577) that encodes one or more of the enzymes in the methylmalonyl-CoA pathway as described herein. In some embodiments of any of the aspects, the probiotic bacteria comprises at least a portion of the genome of V. dispar (see e.g., RefSeq: NZ_ACIK00000000.2, genome assembly ID 172077, available on the world wide web at ncbi.nlm.nih.gov/genome/2066?genome_assembly_id=172077) that encodes one or more of the enzymes in the methylmalonyl-CoA pathway as described herein. In some embodiments of any of the aspects, the probiotic bacteria comprises at least a portion of the genome of V. parvula (see e.g., RefSeq: NC_013520.1, genome assembly ID 172451, available on the world wide web at ncbi.nlm.nih.gov/genome/2471?genome_assembly_id=172451) that encodes one or more of the enzymes in the methylmalonyl-CoA pathway as described herein.

In some embodiments of any of the aspects, the probiotic bacteria comprises a strain selected from the group consisting of Veillonella atypica KON, Veillonella dispar ATCC 17748, and Veillonella parvula DSM 2008.

### Lactate Metabolism

In some embodiments, the Veillonella probiotic bacterium comprises and expresses genes encoding enzymes sufficient to metabolize the conversion of lactate into short-chain fatty acids (SCFAs) including one or more of propionate and acetate. In some embodiments of any of the aspects, the Veillonella probiotic bacterium comprises and expresses genes encoding enzymes from the methylmalonyl-CoA pathway.

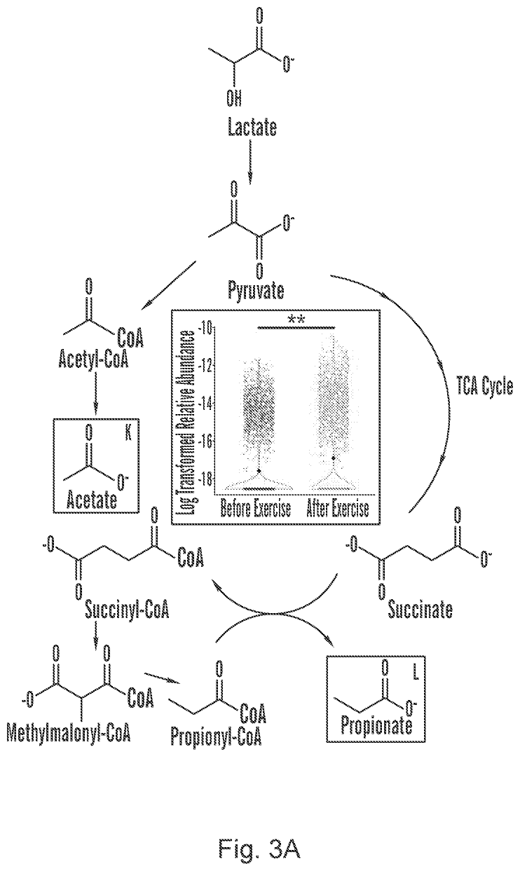

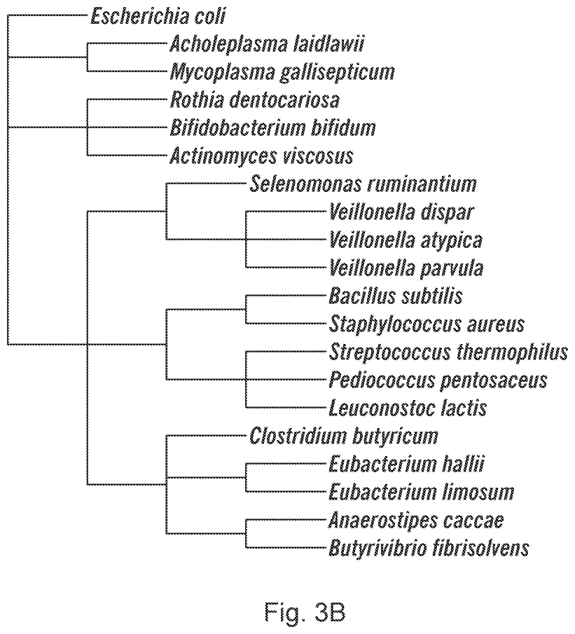

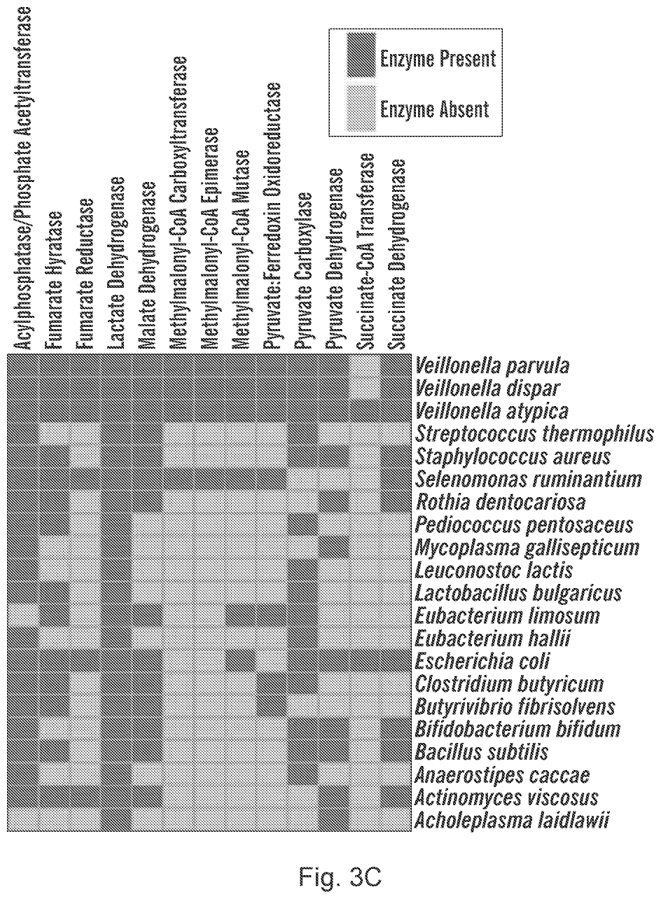

The methylmalonyl-CoA pathway is a pathway whereby lactate can be converted into SCFAs comprising one or more of propionate and acetate (see e.g., FIG. 3A). In some embodiments, genes encoding enzymes from the Veillonella methylmalonyl-CoA pathway can comprise Acylphosphatase/Phosphate Acetyltransferase (see e.g., VPAR_RS08775); Fumarate Hydratase (see e.g., VPAR_RS08475, VPAR_RS07815, VPAR_RS07810; fumarate hydratase can also be referred to herein as fumarase or a class II fumarate hydratase); Fumarate Reductase (see e.g., VPAR_RS08720; fumarate reductase can also be referred to herein as succinate dehydrogenase or fumarate reductase, flavoprotein subunit); Lactate Dehydrogenase (see e.g., VPAR_RS02525, VPAR_RS08165; lactate dehydrogenase can also be referred to herein as 2-hydroxyacid dehydrogenase); Malate Dehydrogenase (see e.g., VPAR_RS02200); Methylmalonyl-CoA Carboxyltransferase (see e.g., VPAR_RS06275); Methylmalonyl-CoA Epimerase (see e.g., VPAR_RS06280); Methylmalonyl-CoA Mutase (see e.g., VPAR_RS09005, VPAR_RS09000, VPAR_RS06290, VPAR_RS06295); Pyruvate:Ferredoxin Oxidoreductase (see e.g., VPAR_RS07065; Pyruvate:Ferredoxin Oxidoreductase can also be referred to herein as PFOR, pyruvate synthase, or pyruvateferredoxin (flavodoxin) oxidoreductase); Pyruvate Carboxylase (see e.g., VPAR_RS03795); Pyruvate Dehydrogenase (see e.g., poxB, NCTC11831_00991, LR134375 1054410-1056152; pyruvate dehydrogenase can also be referred to herein as thiamine pyrophosphate enzyme or TPP binding domain protein; pyruvate dehydrogenase can be a ubiquinone-dependent pyruvate dehydrogenase); Succinate-CoA Transferase (see e.g., VPAR_RS06300; succinate-CoA transferase can also be referred to herein as acetyl-CoA hydrolase); and Succinate Dehydrogenase (see e.g., VPAR_RS08720). The nucleotide sequence for a gene encoding an enzyme from the Veillonella methylmalonyl-CoA pathway can also comprise any nucleotide sequence that when translated exhibits identity with one of the amino acid sequences described herein.

In some embodiments, enzymes from the Veillonella methylmalonyl-CoA pathway comprise Acylphosphatase/Phosphate Acetyltransferase (see e.g., NCBI Accession Numbers KXB88934.1, KXB87671.1, KXA63917.1); Fumarate Hydratase (see e.g., NCBI Accession Numbers RJY50419.1, RIW10507.1, RYS56734.1, KXB85322.1; fumarate hydratase can also be referred to herein as fumarase); Fumarate Reductase (see e.g., NCBI Accession Numbers ACZ25381.1, EEP66133.1, EFL57478.1; fumarate reductase can also be referred to herein as succinate dehydrogenase or fumarate reductase, flavoprotein subunit); Lactate Dehydrogenase (see e.g., NCBI Accession Numbers ACA84166.1, WP_009353153.1; lactate dehydrogenase can also be referred to herein as 2-hydroxyacid dehydrogenase); Malate Dehydrogenase (see e.g., NCBI Accession Numbers AGE34478.1, PKZ93037.1, ARF99954.1); Methylmalonyl-CoA Carboxyltransferase (see e.g., NCBI Accession Number WP_062401066.1, RYS57645.1, WP_126939950.1); Methylmalonyl-CoA Epimerase (see e.g., NCBI Accession Numbers ACZ24925.1, KXB87909.1, KXA62259.1); Methylmalonyl-CoA Mutase (see e.g., NCBI Accession Numbers KXB88884.1, KXA61177.1, KXB85016.1); Pyruvate:Ferredoxin Oxidoreductase (see e.g., NCBI Accession Numbers KXB85069.1, KXB85069.1, KXB83500.1, ACZ25068.1; Pyruvate:Ferredoxin Oxidoreductase can also be referred to herein as PFOR, pyruvate synthase, or pyruvate:ferredoxin (flavodoxin) oxidoreductase); Pyruvate Carboxylase (see e.g., NCBI Accession Numbers KXB88195.1, KXB84281.1, KXA65529.1); Pyruvate Dehydrogenase (see e.g., NCBI Accession Numbers WP_060924568.1, WP_060919818.1, WP_060807170.1, VEG93557.1; pyruvate dehydrogenase can also be referred to herein as thiamine pyrophosphate enzyme or TPP binding domain protein; pyruvate dehydrogenase can be a ubiquinone-dependent pyruvate dehydrogenase); Succinate-CoA Transferase (see e.g., NCBI Accession Numbers KXB87913.1, KXB86203.1, ACZ24929.1, EKY19761.1; succinate-CoA transferase can also be referred to herein as acetyl-CoA hydrolase); and Succinate Dehydrogenase (see e.g., NCBI Accession Numbers ARG00060.1, KUH50526.1). The amino acid sequence for an enzyme from the Veillonella methylmalonyl-CoA pathway can also comprise any amino acid sequence translated from one of the nucleic acid sequences described herein.

In some embodiments, the presence of gene encoding an enzyme from the methylmalonyl-CoA pathway in the genome of a Veillonella species can be determined by using NCBI BLAST keyword or sequence searches or can be detected experimentally using gene-specific primer sequencing or detection of genomic DNA (see e.g., FIG. 3C). In some embodiments, the expression of an enzyme from the methylmalonyl-CoA pathway can be detected experimentally using an assay including but not limited to PCR or RT-PCT. In some embodiments, the activity of an enzyme from the methylmalonyl-CoA pathway can be detected experimentally using an assay including but not limited to mass spectrometry analysis of enzyme reactions.

As described herein, Veillonella bacteria comprise at least 12 of the 13 methylmalonyl-CoA pathway enzymes sufficient for conversion of lactate into propionate and acetate (see e.g., FIG. 3C). As a non-limiting example, the reference genome on NCBI for V. atypica has been shown to comprise all 13 of the 13 methylmalonyl-CoA pathway enzymes sufficient for conversion of lactate into propionate and acetate (see e.g., FIG. 3C). As a non-limiting example, the reference genomes on NCBI for V. parvula and V. dispar have been shown to comprise 12 of the 13 methylmalonyl-CoA pathway enzymes sufficient for conversion of lactate into propionate and acetate, with apparent absence of the Succinate-CoA Transferase gene (see e.g., FIG. 3C). However, this apparent absence of the Succinate-CoA Transferase gene in the genomes of V. parvula and V. dispar is an annotation error as production of propionate was validated via mass spectrometry on isolates of V. parvula and V. dispar. As shown herein, Veillonella species comprise and express enzymes sufficient to metabolize lactate into the SCFAs acetate and propionate via the methylmalonyl-CoA pathway (See e.g., Table 1).

In some embodiments of any of the aspects, a probiotic bacterium as described herein encodes and expresses (or is engineered to encode and express) comprises at least one lactate metabolism gene as described herein. Relevant expression is that which occurs in the human gut or under conditions that occur in the human gut, e.g., in the colon or small intestine. In some embodiments the probiotic bacteria expresses at least 1 enzyme, at least 2 enzymes, at least 3 enzymes, at least 4 enzymes, at least 5 enzymes, at least 6 enzymes, at least 7 enzymes, at least 8 enzymes, at least 9 enzymes, at least 10 enzymes, at least 11 enzymes, at least 12 enzymes, or at least 13 enzymes lactate metabolism enzymes as described herein, e.g., of the methylmalonyl-CoA pathway.

In some embodiments of any of the aspects, the nucleic acid sequence encoding the lactate metabolism enzyme comprises one of SEQ ID NO: 8-15 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NO: 8-15 that catalyzes the same lactate metabolism reaction.

SEQ ID NO: 8, V. atypica Fumarate Hydratase 1, 1356 nt
ATGGAAACAAGAATTGAATATGATTCAATGGGACCAGTCGAAGTCGACGCTAGACGAAT
TTACGGACCTCAAACGCAACGCTCGTTCAATAACTTTAAGATCGGTGACCACCGCATCCC
TATTGAACAAATCAAAGCGCTTGCGCTTGTCAAAAAAGCATGTGCTTTAACCAATGCAAA
ATGTGGCGCAGTAACTGAGGAAAAAGCGAAACTCATTGCCCAAGTAGTAGATGAAATCG
TAGATGGCAAATGGGATGAGGAATTCCCATTGACCGTATTCCAAACAGGCTCTGGCACA
CAAACAAACATGAATGTGAACGAAGTTATCGCTCACCGGGCTAAGCAGTTAGATGAAGC
TAATCCACTTCATCCTAACGACGATGTAAACCGCGGCCAAAGTACAAATGATACATTCCC
AACAGCAATGCATATCTGCGGGTACTTTGAAATTACAAAACGCGTAATTCCTGCATTGGA
CGGCCTTATTGTATCCTTTGAAAAATTGCAAGAAAAAGGTATAGGCTTGCAAAAGGTTGG
TCGTACTCACCTACAAGATGCTACTTTCATCATGGTGGATCAAGAAATTAGCGCCTTTGT
AGACGGCCTAAAAACTGCCAAAACTATGCTCCTTCAAAACGCTGACTACCTACTCGACGT
TGCCCTTGGCGGCACTGCCGTTGGTACAGGTGTGAACACACCTAAGGGATATCTAGACGT
TATGGAAACCGTATTGCCAGAAGTAACAGGCGCTCCATTCCGCGTTAAGAATAATAAATT
CCAAGGCCTATCCTTAAAAGATGCGTTCATGATGGCTCACGGCGCACTTAACACATTGGC
TACTACATTGTTTAAGATTGCGAACGATGTCCGTTTCTTAGGATCCGGCCCTCGCTGTGGC
TATGGCGAATGGCATCTTCCTGAAAACGAACCAGGCTCCTCCATCATGCCAGGTAAGGTT
AACCCTACCCAATGCGAAGCTCTTGCTATGGTATGTGCCCAAGTATTCGGTATCAATACA
ACCATGACACTTTGTGCAGGTAGCGGTGCGTTCCAATTGAACGTGTACATGCCTATCATG
ATCTATGATTTTGTTGAAAGCTGTCGCCTCCTCGCAGATGCGATGAATTCCTTCACTACAC
ACTGCATCGACGGCGTAGAATTCGTACCTGAAAAACTTAAATTCTTCGTAGAACAATCCC
TTATGATTGCTACTTCCTTAACACCATACATCGGCTACGATAAGAGTGCTAAGGTCGTAA
AAGAAGCATACAAACGCGGTTGCTCTATTAAAGAAATCATCTTGGAAGAAAAACTCATG
ACCGAAGACGAATTTGCTGAAGCAGTTCGCATGAAATAA
SEQ ID NO: 9, V. atypica Fumarate Hydratase 2, 843 nt
TTGCGCGAGATTCAAGTATCTGAAATCACAAAAACAGTCCGTCAAATGTGTATGGACGC
AGCTTACCACTTGCCAAAAGACATCTATGAAGGCTTGAAAAAAGGCCGTGAAACAGAAG
AGTCTCCAGTTGGTTGCATCGTTCTCGATCAAATCATCAAAAATGCAGAAATTGCTGATG
CTGAAGATCGTCCATACTGCCAAGATACTGGTATGACTATGGTATTCTTAAAAGTTGGTC
AAGATGTTCATTTCGTTGGTGGTGATCTTACAGAAGCTATCAATGCTGGTGTTGCAGCTG
GTTATGTTGAAGGTTATCTTCGTAAATCCGTTGTAGCTGAACCATTGTTCAATCGTAAAA
ATACACAAAATAACACACCTGCAATCATCTATACTGAAATCGTTCCTGGCGATAAAGTAG
ATATTCAAGTAGAATTAAAAGGTTTCGGTTCTGAAAATAAATCCGATGTAGCTATGCTCG
TACCTGCTGATGGTGTGGAAGGCGTTAAAAATGCAGTCCTTGAAATCGTAAAACATGCA
GGTCCTAACCCATGCCCTCCAATTGTACTCGGCATTGGTATTGGCGGTACTATGGACCAA
GCAGCTGTTATGTCCAAAAAAGCGTTGCTTCGTGACATTAGCGTACCTCATAAAGATGCA
GACTATGCAAAATTAGAAGAAGAAATCATGGAAATGGTTAACAAAACTGGTATTGGACC
ACAATTGGGTGGCACTACAACTTGTATCGGTGTAAACATCGAATGGGGTGCAACTCACAT
CGCAGGTCTTCCTGTTGCAGTTACTATCATGTGCCATGCTGCTCGTCATGCTCACGTAGTA
CTTTAA
SEQ ID NO: 10, V. atypica Fumarate Reductase, 963 nt
ATGAGACAAATCACCTATCATATCCACCGTTACCAACAAGGTCGCGCGTTTGTACAAACT
TTCAAATTCGACTATGAAGCTGACCGTACAATTCTTTGGGGCCTTCAAAAAATTAAAGAT
ACTCAAGATCCAACATTAACATTCTTGGCAGCTTGTCGTTCCGCAGTTTGTGGCGCTTGCT
CCATTCGCGTAAATGGCGAAGCAATGCTTGGTTGTGAATCTAAAATTGATGAATTGACAG
AACGTTATGGTACAGATGAATTAACTATCGCTCCTATCGGTAACTTCCGTGTAATCCGCG
ACTTAGTTGTTGATTGGGAATCTAAAGTTGATCGTTTGAAAACAGTTGCTCCTTGGATTTT
CCTTAAAGCAGAATTCAACGAAGGCGACAAAATCGTTCGTCAAACTCCAGCTGATTTCA
AAAAATTCGTTGCTGGTACAGAATGTATCCTTTGCGGTTGCTGCGCATCTGAATGTAACA
AATTGACTGCACGTCAAGACGATTTCTTGGAACCATATGTATTCACTAAAGCTAACCGTT
TCGTATTGGATAGCCGTGATGATGCACCTATGGCTCACATTCAACCTGCTTATGACAATG
GTCTTTGGAAATGCGTTCACTGCATGAACTGTATCTCCCGTTGTCCTAAACACTTAAAAC
CTGCTCAAGATATTTCCAACATGCGTAAAGAAGCTACAAAAGCTGGCCTTACAAACAAC
AAAGGCGTTCGCCATGCAGTTGCTTTCAAAGAAGACCTTTACAAAACTGGTCGTTTGAAA
GAAGTTTCTATGAGCTTGAAATCTGACGGTGTTGTAGATTCCGCTAAACAAGCATTCTAT
GCATTACGTTTGTGGAAACACAGCAAAATTAATCCTTTCGAACTTGTAGTACCTCAAAAA
CCAGTTAATGGTATTGATGGTGTTCGCAGACTTATGAAAGCAGCTGAGGAGGTAAGCAA
ATAA
SEQ ID NO: 11, V. atypica L lactate dehydrogenase, 948 nt
ATGAAATTACGTAAAGTAGGTATTATCGGAACTGGACATGTAGGCTCGCATGTAGCCTTT
TCTTTAGCTTTGCAAGGCGAAGTTGATGAGTTATATATGATGGATATTGATGAAAAGAAA
GCTAAAGCACAGGCTATGGATGTTAATGATGCGGTTAGTTATATACCTCATCGTGTAACG
GCTACATCTGGTCCGATTGAAGACTGTGGTGATTGTGATATTCTTGTTTTTAGTGCAGGTC
CACTACCTAATTTATATCAAGATCGCCTAGAAAGCCTTGGTGATACAATCGCAGTACTAA
AGGATGTTATTCCTCGCATTAAAGCATCTGGATTTAAGGGCTTTATTATTTCTATATCTAA
TCCAGCAGATGTAGTGGCTACGTATTTATGTAAACATTTAGATTGGAATCCAAAGCGCAT
TATTTCGTCAGGTACAGCGCTAGATTCTGCAAGATTGCAAAAAGAGTTAGCTCATATATT
CGATATTAGCAATCGAACTATTACTGCTTATTGCATGGGTGAGCATGGCGCAAGTGCTAT
GGTGCCATGGTCCCATGTGTATGTACAAGGCAAGCCGTTAGTAGAATTGCAAAAAGAAT
TGCCTCATAGATTTCCAGAACTAGATCATAAACAAGTGTTAGATGATGTTAAAATTGGTG
GATATCATGTGTTGGCAGGCAAAGGCTCTACGGAATTTGGTATAGCAAGTGCCACCACA
GAGTTAATTCGCTCTGTATTCCATGATGAGAAAAAAGTATTGCCATGTTCTTGTTATTTAG
ATGGTCAATACGGGGAAACAGGCGTTTTTGCATCCACACCTGCTGTCATTGGCAAGGATG
GCATAGAAGATGTTCTTGAATTACAAATGACAGAGGATGAATTGGCTTTATTTAAGAAAT
CCTGTGCGGTTATTAGAGAATATGCAAAAAAAGCAGAAACTATGTAA
SEQ ID NO: 12, V. atypica Methylmalonyl CoA Carboxyltransferase,
1530 nt
ATGCAAACAGTGCAAGAAAAAATTGAGTTGTTGCACGAAAAACTAGCAAAAGTTAAAGC
TGGTGGCGGTGAAAAACGCGTTGAGAAACAACATTCTCAAGGTAAAATGACTGCTCGTG
AACGTTTGGCTAAATTATTCGATGACAACTCTTTTGTTGAACTTGATCAATTCGTTAAACA
TCGTTGTGTTAACTTCGGTCAAGACAAAAAAGAATTACCAGGCGAAGGTGTAGTAACTG
GTTATGGTACTATCGATGGTCGTTTGGTATATGCATTCGCACAAGACTTCACTGTAGAAG
GTGGTTCTCTTGGTGAAATGCACGCTGCTAAAATCGTTAAAGTACAACGTTTAGCAATGA
AAATGGGTGCTCCTATCGTTGGTATCAATGATTCCGGCGGGGCTCGTATTCAAGAAGCTG
TAGATGCTCTTGCTGGTTACGGTAAAATTTTCTTTGAAAATACAAATGCATCTGGCGTTAT
TCCACAAATTTCCGTAATCATGGGACCATGTGCAGGCGGTGCTGTATATTCTCCAGCATT
GACTGACTTCATCTACATGGTTAAAAACACATCTCAAATGTTCATCACTGGTCCTGCAGT
TATTAAATCTGTAACTGGTGAAGAAGTAACAGCTGAAGATCTTGGTGGCGCAATGGCTC
ACAACTCTGTGTCTGGTGTTGCTCACTTCGCAGCTGAAAATGAAGATGATTGCATCGCTC
AAATCCGCTACTTGTTAGGTTTCTTACCTTCTAACAACATGGAAGATGCTCCATTGGTAG
ATACAGGTGATGACCCAACTCGTGAAGATGAAGGCTTAAACAGCTTGTTACCTGATAAC
AGCAACATGCCTTACGACATGAAAGATGTTATCGCAGCTACTGTAGATAATGGCGAATA
CTATGAAGTACAACCATTCTATGCTACAAACATCATTACATGCTTCGCACGTTTTGATGG
TCAATCTGTTGGTATCATTGCTAACCAACCTAAAGTAATGGCTGGTTGCTTGGACATCAA
CGCATCTGATAAATCTTCCCGTTTTATCCGTTTCTGTGATGCTTTCAATATTCCAATCGTT
AACTTCGTTGACGTTCCTGGTTTCTTGCCTGGCACAAATCAAGAATGGGGCGGTATCATT
CGTCATGGTGCTAAAATGTTGTATGCTTACTCTGAAGCAACAGTACCAAAAATTACTGTT
ATCACTCGTAAAGCATACGGTGGTTCTTACCTCGCTATGTGTTCCCAAGATTTGGGCGCT
GATCAAGTATACGCTTGGCCTACATCCGAAATCGCTGTAATGGGTCCTGCTGGTGCAGCT
AACATCATCTTCAAAAAAGATGAAGATAAAGACGCTAAAACAGCTAAGTACGTAGAAGA
ATTCGCAACTCCTTACAAAGCTGCAGAACGTGGCTTCGTTGATGTTGTAATCGAACCAAA
ACAAACTCGTCCAGCTGTTATCAATGCGTTGGCTATGCTTGCAAGTAAACGTGAAAACCG
TGCTCCAAAGAAACATGGTAATATTCCATTATAA
SEQ ID NO: 13, V. atypica Methylmalonyl CoA Epimerase, 423 nt
ATGGCTTTTAAAGTATTACAAGTGGATCACATCGGTATTGGTGTTAATGATTTAGCAGCA
ACTAAAGAATTTTACAAAAATGCTTTGGGAATTCAACATCTTCCTGAAGATGAAGTAGTA
GAAGAACAAAAAGTAAAAGTATCCTTCTTCCCATGCGGCGATGCTGAATTAGAATTCTTG
GAAACTACTACTCCAGACGGCCCTATCGGTAAATTCATCGAAAAAAATGGCGGTCGTGA
TGGTATCCAACACGTTGCTTTGCGTGTAGATAATATTGAAAATGCTATTGCTGATCTTATG
GCGAAAGGTATTCGTATGATTGACGAAAAACCTCGTTATGGTGCTGGCGGTTCCTCTATT
GCATTCGTTCATCCTAAAGCTACAGGTGGCGTATTACTTGAACTTTGTCAACGAATGAAA
TAA
SEQ ID NO: 14, V. atypica Methylmalonyl CoA Mutase, 2190 nt
ATGTTTAAAAATCCAGACTTCTCCTCTCTTGGCTTGGGATCTCAGTCTGCCACATCGCGCG
ATGCATGGCTTGCTGAACTTAAAAAAGAAACAGGAAAAAGCTTTGAAGACTTATATAAC
ACGACAATGGAGCAAATCCAATTAAAACCTCTCTATACAGAGATGGATTATGAAGGGAT
GACACACCTTGACTATATGGCTGGTGTACCTCCATTCTTGCGTGGACCTTATTCCACAATG
TACGTAACTCGTCCTTGGACAGTGCGTCAGTACGCTGGTTTCTCTACAGCCGAAGAATCC
AATGCGTTCTATCGTCGTAACTTGGCAGCAGGCCAAAAAGGGTTGTCTATTGCCTTTGAC
CTTGCTACTCACCGTGGTTATGACTCCGACCATCCTCGCGTAGTGGGCGACGTTGGTAAA
GCGGGCGTTGCGGTAGACTCCATCCTCGATATGGAAATTCTATTCTCTGGTATCCCTCTTG
ACCAAATGTCCGTATCCATGACAATGAACGGCGCCGTATTGCCTGTTATGGCATTCTACA
TCCTAGCTGGTGAAGAACAAGGTGTTGATAAAAAGGTTATGGCCGGTACAATCCAAAAT
GATATCTTGAAAGAGTTCATGGTACGTAATACCTATATTTACCCTCCAGCTACGTCCATG
CGCATCATCGGTGATATCTTTGCGTATACATCCCAGAACATGCCTAAATTCAACAGTATC
TCTATTTCTGGCTACCATATGCAAGAAGCGGGTGCAACGGCGGATATCGAATTAGGTTAT
ACATTGGCTGACGGCCTTGAATACATCCGTACCGGTGTAAATGCAGGTCTTCATGTTGAC
CAATTCGCACCTCGTTTGTCCTTCTTCTGGGCTATCGGCAAAAACTACTTTATGGAAGTGG
CTAAAATGCGTGCGGCTCGTATGTTGTGGGCTAAGATTATTAAGAGCTTCGGTTCTGAAA
ATCCTAAATCTATGGCTCTTCGTACACATAGCCAAACATCTGGTTGGTCTCTTACAGAAC
AAGATCCATTCAACAACGTGGCTCGTACATGTATGGAAGCGATGGGCGCCGCATTGGGC
CATACGCAATCCCTACATACGAATGCGCTTGATGAAGCCATCGCGTTACCTACAGACTTC
TCTGCACGTATTGCGCGTAATACACAACTTTACATCCAAGATGAAACTAAGGTATGTAAG
GTTATCGACCCATGGGGCGGTTCCTACTATGTAGAAGCATTGACTGACGAATTGATCCGT
CGTGCCTGGGGCCATATCCAAGAAATCGAATCCCTTGGTGGTATGGCGAAAGCGATTGA
CACGGGTCTTCCTAAGATGCGTATCGAAGAGGCGGCAGCTCGTCGCCAAGCTCGTATCG
ACTCTGGTCGTGAAGCTATCGTCGGCATCAATAAATATCGTCTAGATAAAGAAGATCCAT
TGGATATCCTCGATGTAGATAATACAGCTGTACGGGAAGCGCAAATCCGTCGTCTCGAAC
AATTGCGTGCTAACCGTGATGAAGACAAGGTTCAATCCTGCCTCGAAGCGATTACAAAT
GCTACCGAATCTGGTGAAGGCAACTTGCTTGCCCTTGCACTTGAAGCAGCTCGTGCTCGT
GCATCTTTGGGCGAAATTTCCTTTGCTGTTGAAAAAGTTTGTGGCCGTCATAAAGCGGTT
ATCCGCTCTATTTCCGGTGTATACTCCAGCGAATATGAAGATGATGATGTAATCAAAGAA
GTACGTCAAATGGCAGATGACTTTGAAGAACTCGAAGGTCGTCGCCCTCGTATCATGATT
GCTAAGATGGGCCAAGACGGTCATGACCGCGGTGCCAAAGTTATTGCTACATCCTTCGCG
GATATGGGCTTTGACGTGGATATCGGACCTTTGTTCCAAACTCCAGAAGAAACAGCACA
AGATGCGGTGGATAATGACGTTCACATCGTCGGATTTAGTTCTCTTGCAGCAGGTCACAA
AACATTGTTACCTCAACTTGTAGAAGAACTCAACAAGCGAGGTCGTGGAGATATTTTAGT
TGCCATCGGCGGCGTAATCCCTGCTCAGGACTATGAGTTCCTACGCGAACATGGCGCAGT
GGCTATCTTTGGCCCTGGTACAGTTTTGCCAGTAGCGGCGAAAAAATTACTAGAAACATT
GACTAGCCACGTTCAAGACGAAGGCAATGACTGA
SEQ ID NO: 15, V. atypica Pyruvate Decarboxylase, 3447 nt
ATGAAGAAAATTAAATCCGTTTTGGTAGCCAATCGTGGCGAAATCGCAATCCGCGTATTT
CGTGCATGTAACGAAATGGGTATTAAAACAGTAGCTATCTATTCTAAAGAAGACACATT
GTCCTTGCACCGTAACCAAGCTGATGAAGCATATTTGGTTGGGGAAGGTAAAAAACCAG
TTGATGCCTATTTGGATATTGAAGATATCATCCGCATTGCTAAAGAGCATGACATTGATG
CTATCCATCCTGGCTATGGTTTCTTATCCGAAAACGAAGAGTTCGCTCGTCGTTGCGGTG
AAGAAGGTATCATTTTCATTGGACCTCACGTAGAACATTTGAATATGTTCGGCGACAAGG
TTAATGCTCGTACACAAGCTAAATTGGCAGACATTCCAATGATTCCAGGTTCTGATGGGG
CATTGCGTGATTTTGCACAATTAGAAGAATTTGCTGAAACTCACGGCTATCCATTGATGA
TTAAAGCCGTAAACGGTGGTGGCGGTCGTGGTATGCGCGAAGTTCACCGCAAAGAAGAT
TTACGCGATGCTTATGACCGCGCTAAATCCGAAGCTAAAGCAGCATTTGGTGATGACGAT
GTTTACGTAGAAAAACTCATCGTTGAACCTAAACATATTGAAGTACAAATCTTAGGTGAT
GAACATGGTAATGTAGTTCATTTACATGAACGTGACTGCTCTGTACAACGTCGTCACCAA
AAGGTTGTTGAAATGGCACCAGCTTTTGCATTGCCATTAGAAACTCGTAAAGCCGTATGT
GATGCAGCCGTAAAAATCATGAAAAATGTTGGCTACGTTAATGCTGGTACTGTTGAATTC
TTGGTAACTTCTGATGGTTCCTTCTATTTCATCGAAGTTAACCCTCGTATCCAAGTAGAAC
ATACCGTAACAGAAATGATTACGGACATCGATATTGTTCATTCTCAAATTCGTATCGCTG
AAGGCTATGATTTGCACAGCCCAGAAATAGGCATTCCTGAACAAGATGAAATTCCTTGTA
AAGGTACTGCAATTCAATGTCGTATCACTACAGAAGATCCTAAGAACAACTTTATGCCAG
ATACTGGTAAAATCTTAGCATATCGTAGTTCCGGCGGCTTTGGTATTCGCCTTGACTCTGG
TAATGCTTTCACAGGCGCTGTAGTAACACCTTATTATGATTCCCTATTGGTAAAAGCTACT
GCATTCGGTCCTAACAACGAAGAAACTATTCGTAAAATGCTTCGTTGCTTGAAAGAATTC
CGTATTCGTGGCGTTAAAACAAACATTCATTTCTTGATTAACGTTCTTGAAAACCCTGAA
TTCCAAAGCGGTAACTACACAGTTAACTTCATTGAAGACCATCCTGAATTATTCGAATTA
AAACCAGATCGCGACCGTGGCACTAAATTGCTTCGCTACATTGCGGATACTACAATCAAT
GGTTACTCCGGTGCAGGCCCTCAAGAGGTACCTGATTTTGAACCAATGCAATTGCCATCT
AAATTAGATGTATCTCCTGCACCTGGTACAAAACAAAAATTCGACGAATTAGGTCCAGA
AGGTTTCAGTAAATGGTTGGCTGACCAAAAACAAGTATTCTTTACAGATACAACATGGCG
TGATGCTCACCAATCCTTATTTGCTACACGTTTGCGTACCATCGATATGGCACGCGTAGCT
GGCGATGCCGCTAAAGGTGTACCAAATCTATTCTCCTTAGAATGTTGGGGCGGTGCAACA
TTTGACGTATCCTATCGCTTCTTGCACGAAGATCCATGGGAACGTTTGCGCATGTTCCGTA
AAGAAGTGCCTAATACATTGCTTCAAATGTTGATTCGCGGTGCTAATGCCGTTGGTTATA
CATCCTATCCTGATAATGTAGTTCGTAATTTCATTCAATTGTCCGCTAAAAATGGTATCGA
CGTATTCCGCGTATTTGATAGCTTGAATAGCCTTGACAATATGAAGGTTGCTATTGATGA
AGTTCGCAATCAAAATAAGATTGCTGAAGTTGCATTGTGCTACACAGGCGATATCCTCGA
CAGCAATCGTCCTAAATACAATCTTGATTACTATGTAAAAATGGCTAAAGAATTGCAAAA
TGCTGGTGCCAATATCATTGCTATTAAAGATATGGCTGGTTTGTTGAAACCTCAAGCTGC
ATACAACTTAGTATCCGCTTTGAAAGATGCTGTAACAGTACCAATTCATTTGCATTCTCAT
GAAGGTTCTGGCAATACTATTTATTCTTATGGACGTGCTGTAGATGCTGGTGTTGACGTT
ATTGACTTAGCATATTCTGCATTTGCTAATGGCACTAGCCAACCAAGCATGAATTCTATG
TATTACGCTTTAGCTGGTACTGAACGTCAACCACAAATGAATATTGACTACATGGAAGAA
ATGTCTCATTACTTCGGTAGTATTCGTCCTTACTACAGAGGCGTTGATAAAGCTGAAAAA
TATCCAAATACAGAAGTATACCAACATGAAATGCCAGGCGGTCAATATTCCAACTTGCA
ACAACAAGCTAAAATGGTTGGACTTGGTGATCGTTGGACAGACATTAAAAAAGTATATC
ACCAAGTTAATATGATGTTTGGTGATATCATCAAGGTAACACCTTCTTCTAAAGTCGTTG
GTGATATGACATTGTACATGGTACAAAATAATTTGACTGAAAAAGATATCTATGAAAAA
GGTGATACTCTTGATTTCCCTCAATCAGTAGTAGAATTCTTCGAAGGTCGTCTAGGTACA
CCATATCAAGGGTTCCCTGAAGAACTTCAAAAAATCATCTTGAAAGGTGCTCGCCCTATT
ACTGTTCGTCCTGGTGCTGTATTACCTCCAACTGATTTCGAACATGTTCGTAATGAATTAA
ACGAAATGGGTGCTAATACTACTGATGAAGATGTAAGTGCATACTGCTTGTATCCTAAAG
TATTTAAAGACTACACTAAATTTACTAAAGACTTTGGTAATGTATCTGTACTAGATACAC
CAACATTCTTCTTTGGTATGAAACGTGGTGAAGAAATTCAAGTTACTATTGAAAAAGGTA
AAACATTAATTATCAAGATGAATGGTGTATCCGATCCTGATGAAGATGGTAATCGCATCG
TTCTCTTTGAATTCAATGGCCAACCACGTTCTATTAAAGTTCATGATAAACATGCTAAAA
CAACTGGCGTTGTTCGTCGTAAAGTAAATGAATCCAACCCTGGTGAAATTGGTGCTACGT
TGTCTGGCTCCGTTGTTAAGATTCTAGTTAAGAAAGGTCAATCCGTAACTAAAGGTGAAC
CATTAATCGTAACAGAAGCTATGAAGATGGAAACAACAATTACAGCTCCTATCGGTGGT
ATCGTTGAAGAAATTCTTGTTCGTGAAGGCAGCCGTATCGAATCCGGTGATTGCTTGTTA
CACATTGAAGATGCTATTAAACGTTAA

In some embodiments of any of the aspects, the probiotic bacteria expresses or is engineered to express a polypeptide encoded by a lactate metabolism gene. In some embodiments of any of the aspects, the polypeptide encoded by a lactate metabolism gene comprises SEQ ID NO: 16-23 or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% similar to the sequence of one of SEQ ID NO: 16-23 that catalyzes the same lactate metabolism reaction.

SEQ ID NO: 16, V. atypica Fumarate Hydratase 1, 451 amino acids (aa)
METRIEYDSMGPVEVDARRIYGPQTQRSFNNFKIGDHRIPIEQIKALALVKKACALTNAKCGA
VTEEKAKLIAQVVDEIVDGKWDEEFPLTVFQTGSGTQTNMNVNEVIAHRAKQLDEANPLHP
NDDVNRGQSTNDTFPTAMHICGYFEITKRVIPALDGLIVSFEKLQEKGIGLQKVGRTHLQDAT
FIMVDQEISAFVDGLKTAKTMLLQNADYLLDVALGGTAVGTGVNTPKGYLDVMETVLPEVT
GAPFRVKNNKFQGLSLKDAFMMAHGALNTLATTLFKIANDVRFLGSGPRCGYGEWHLPENE
PGSSIMPGKVNPTQCEALAMVCAQVFGINTTMTLCAGSGAFQLNVYMPIMIYDFVESCRLLA
DAMNSFTTHCIDGVEFVPEKLKFFVEQSLMIATSLTPYIGYDKSAKVVKEAYKRGCSIKEIILE
EKLMTEDEFAEAVRMK
SEQ ID NO: 17, V. atypica Fumarate Hydratase 2, 280 aa
MREIQVSEITKTVRQMCMDAAYHLPKDIYEGLKKGRETEESPVGCIVLDQIIKNAEIADAEDR
PYCQDTGMTMVFLKVGQDVHFVGGDLTEAINAGVAAGYVEGYLRKSVVAEPLFNRKNTQN
NTPAIIYTEIVPGDKVDIQVELKGFGSENKSDVAMLVPADGVEGVKNAVLEIVKHAGPNPCPP
IVLGIGIGGTMDQAAVMSKKALLRDISVPHKDADYAKLEEEIMEMVNKTGIGPQLGGTTTCI
GVNIEWGATHIAGLPVAVTIMCHAARHAHVVL
SEQ ID NO: 18, V. atypica Fumarate Reductase, 320 aa
MRQITYHIHRYQQGRAFVQTFKFDYEADRTILWGLQKIKDTQDPTLTFLAACRSAVCGACSI
RVNGEAMLGCESKIDELTERYGTDELTIAPIGNFRVIRDLVVDWESKVDRLKTVAPWIFLKAE
FNEGDKIVRQTPADFKKFVAGTECILCGCCASECNKLTARQDDFLEPYVFTKANRFVLDSRD
DAPMAHIQPAYDNGLWKCVHCMNCISRCPKHLKPAQDISNMRKEATKAGLTNNKGVRHAV
AFKEDLYKTGRLKEVSMSLKSDGVVDSAKQAFYALRLWKHSKINPFELVVPQKPVNGIDGV
RRLMKAAEEVSK
SEQ ID NO: 19, V. atypica L lactate dehydrogenase, 315 aa
MKLRKVGIIGTGHVGSHVAFSLALQGEVDELYMMDIDEKKAKAQAMDVNDAVSYIPHRVT
ATSGPIEDCGDCDILVFSAGPLPNLYQDRLESLGDTIAVLKDVIPRIKASGFKGFIISISNPADVV
ATYLCKHLDWNPKRIISSGTALDSARLQKELAHIFDISNRTITAYCMGEHGASAMVPWSHVY
VQGKPLVELQKELPHRFPELDHKQVLDDVKIGGYHVLAGKGSTEFGIASATTELIRSVFHDEK
KVLPCSCYLDGQYGETGVFASTPAVIGKDGIEDVLELQMTEDELALFKKSCAVIREYAKKAE
TM
SEQ ID NO: 20, V. atypica Methylmalonyl CoA Carboxyltransferase, 509 aa
MQTVQEKIELLHEKLAKVKAGGGEKRVEKQHSQGKMTARERLAKLFDDNSFVELDQFVKH
RCVNFGQDKKELPGEGVVTGYGTIDGRLVYAFAQDFTVEGGSLGEMHAAKIVKVQRLAMK
MGAPIVGINDSGGARIQEAVDALAGYGKIFFENTNASGVIPQISVIMGPCAGGAVYSPALTDFI
YMVKNTSQMFITGPAVIKSVTGEEVTAEDLGGAMAHNSVSGVAHFAAENEDDCIAQIRYLL
GFLPSNNMEDAPLVDTGDDPTREDEGLNSLLPDNSNMPYDMKDVIAATVDNGEYYEVQPFY
ATNIITCFARFDGQSVGIIANQPKVMAGCLDINASDKSSRFIRFCDAFNIPIVNFVDVPGFLPGT
NQEWGGIIRHGAKMLYAYSEATVPKITVITRKAYGGSYLAMCSQDLGADQVYAWPTSEIAV
MGPAGAANIIFKKDEDKDAKTAKYVEEFATPYKAAERGFVDVVIEPKQTRPAVINALAMLAS
KRENRAPKKHGNIPL
SEQ ID NO: 21, V. atypica Methylmalonyl CoA Epimerase, 140 aa
MAFKVLQVDHIGIGVNDLAATKEFYKNALGIQHLPEDEVVEEQKVKVSFFPCGDAELEFLET
TTPDGPIGKFIEKNGGRDGIQHVALRVDNIENAIADLMAKGIRMIDEKPRYGAGGSSIAFVHP
KATGGVLLELCQRMK
SEQ ID NO: 22, V. atypica Methylmalonyl CoA Mutase, 729 aa
MFKNPDFSSLGLGSQSATSRDAWLAELKKETGKSFEDLYNTTMEQIQLKPLYTEMDYEGMT
HLDYMAGVPPFLRGPYSTMYVTRPWTVRQYAGFSTAEESNAFYRRNLAAGQKGLSIAFDLA
THRGYDSDHPRVVGDVGKAGVAVDSILDMEILFSGIPLDQMSVSMTMNGAVLPVMAFYILA
GEEQGVDKKVMAGTIQNDILKEFMVRNTYIYPPATSMRIIGDIFAYTSQNMPKFNSISISGYH
MQEAGATADIELGYTLADGLEYIRTGVNAGLHVDQFAPRLSFFWAIGKNYFMEVAKMRAA
RMLWAKIIKSFGSENPKSMALRTHSQTSGWSLTEQDPFNNVARTCMEAMGAALGHTQSLHT
NALDEAIALPTDFSARIARNTQLYIQDETKVCKVIDPWGGSYYVEALTDELIRRAWGHIQEIES
LGGMAKAIDTGLPKMRIEEAAARRQARIDSGREAIVGINKYRLDKEDPLDILDVDNTAVREA
QIRRLEQLRANRDEDKVQSCLEAITNATESGEGNLLALALEAARARASLGEISFAVEKVCGRH
KAVIRSISGVYSSEYEDDDVIKEVRQMADDFEELEGRRPRIMIAKMGQDGHDRGAKVIATSF
ADMGFDVDIGPLFQTPEETAQDAVDNDVHIVGFSSLAAGHKTLLPQLVEELNKRGRGDILVA
IGGVIPAQDYEFLREHGAVAIFGPGTVLPVAAKKLLETLTSHVQDEGND
SEQ ID NO: 23, V. atypica Pyruvate Decarboxylase, 1148 aa
MKKIKSVLVANRGEIAIRVFRACNEMGIKTVAIYSKEDTLSLHRNQADEAYLVGEGKKPVDA
YLDIEDIIRIAKEHDIDAIHPGYGFLSENEEFARRCGEEGIIFIGPHVEHLNMFGDKVNARTQAK
LADIPMIPGSDGALRDFAQLEEFAETHGYPLMIKAVNGGGGRGMREVHRKEDLRDAYDRAK
SEAKAAFGDDDVYVEKLIVEPKHIEVQILGDEHGNVVHLHERDCSVQRRHQKVVEMAPAFA
LPLETRKAVCDAAVKIMKNVGYVNAGTVEFLVTSDGSFYFIEVNPRIQVEHTVTEMITDIDIV
HSQIRIAEGYDLHSPEIGIPEQDEIPCKGTAIQCRITTEDPKNNFMPDTGKILAYRSSGGFGIRLD
SGNAFTGAVVTPYYDSLLVKATAFGPNNEETIRKMLRCLKEFRIRGVKTNIHFLINVLENPEF
QSGNYTVNFIEDHPELFELKPDRDRGTKLLRYIADTTINGYSGAGPQEVPDFEPMQLPSKLDV
SPAPGTKQKFDELGPEGFSKWLADQKQVFFTDTTWRDAHQSLFATRLRTIDMARVAGDAAK
GVPNLFSLECWGGATFDVSYRFLHEDPWERLRMFRKEVPNTLLQMLIRGANAVGYTSYPDN
VVRNFIQLSAKNGIDVFRVFDSLNSLDNMKVAIDEVRNQNKIAEVALCYTGDILDSNRPKYN
LDYYVKMAKELQNAGANIIAIKDMAGLLKPQAAYNLVSALKDAVTVPIHLHSHEGSGNTIYS
YGRAVDAGVDVIDLAYSAFANGTSQPSMNSMYYALAGTERQPQMNIDYMEEMSHYFGSIRP
YYRGVDKAEKYPNTEVYQHEMPGGQYSNLQQQAKMVGLGDRWTDIKKVYHQVNMMFGD
IIKVTPSSKVVGDMTLYMVQNNLTEKDIYEKGDTLDFPQSVVEFFEGRLGTPYQGFPEELQKII
LKGARPITVRPGAVLPPTDFEHVRNELNEMGANTTDEDVSAYCLYPKVFKDYTKFTKDFGN
VSVLDTPTFFFGMKRGEEIQVTIEKGKTLIIKMNGVSDPDEDGNRIVLFEFNGQPRSIKVHDKH
AKTTGVVRRKVNESNPGEIGATLSGSVVKILVKKGQSVTKGEPLIVTEAMKMETTITAPIGGI
VEEILVREGSRIESGDCLLHIEDAIKR

### Devices

As described herein, a composition can comprise a device preloaded for administration to a body cavity comprising an effective dose of propionate. In some embodiments, the device can comprise a syringe, an enema, or a suppository. In some embodiments, the propionate can comprise about 10 mM-1000 mM sodium propionate. In some embodiments, the propionate can comprise about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, about 600 mM, about 610 mM, about 620 mM, about 630 mM, about 640 mM, about 650 mM, about 660 mM, about 670 mM, about 680 mM, about 690 mM, about 700 mM, about 710 mM, about 720 mM, about 730 mM, about 740 mM, about 750 mM, about 760 mM, about 770 mM, about 780 mM, about 790 mM, about 800 mM, about 810 mM, about 820 mM, about 830 mM, about 840 mM, about 850 mM, about 860 mM, about 870 mM, about 880 mM, about 890 mM, about 900 mM, about 910 mM, about 920 mM, about 930 mM, about 940 mM, about 950 mM, about 960 mM, about 970 mM, about 980 mM, about 990 mM, or about 1000 mM sodium propionate or any other acceptable salt of propionate. In some embodiments of any of the aspects, the propionate can comprise 150 mM sodium propionate dissolved in PBS. In some embodiments of any of the aspects, the device can be administered rectally, intracolonically, orally, or parenterally.

### Treatment Methods

The compositions described herein can be administered to a subject in need thereof. In some embodiments, the subject can be those who desire improved exercise endurance (e.g., for athletic performance). In some embodiments, the subject can be those with limited exercise endurance (e.g., due to advanced age, other physical impairment, general lack of physical exercise) who can benefit from use of the compositions described.

As described herein, levels of lactate can be elevated in athletes and/or in subjects undergoing exercise. In some embodiments of any of the aspects, the level of propionate can be increased in athletes and/or in subjects undergoing exercise, and or the level of lactate can be decreased in athletes and/or in subjects undergoing exercise. Accordingly, in one aspect of any of the embodiments, described herein is a method of enhancing exercise endurance in a subject in need thereof, the method comprising administering Veillonella probiotic bacteria to a subject in need thereof. Furthermore, in one aspect of any of the embodiments, described herein is a method of enhancing exercise endurance in a subject in need thereof, the method comprising administering propionate to the gut of a subject in need thereof.

In some embodiments of any of the aspects, the method comprises administering Veillonella probiotic bacteria and or propionate to a subject previously determined to have a level of lactate that is high relative to a reference. In some embodiments of any of the aspects, described herein is a method of enhancing exercise endurance in a subject in need thereof, the method comprising: a) first determining the level of lactate in a sample obtained from a subject; and b) then administering a Veillonella probiotic bacteria and or propionate to the subject if the level of lactate is high relative to a reference.

In some embodiments of any of the aspects, the method comprises administering Veillonella probiotic bacteria and or propionate to a subject previously determined to have a level of lactate that is high relative to a reference. In some embodiments of any of the aspects, the step of determining if the subject has a high level of lactate can comprise i) obtaining or having obtained a sample from the subject and ii) performing or having performed an assay on the sample obtained from the subject to determine/measure the level of lactate in the subject. In some embodiments of any of the aspects, the step of determining if the subject has a high level of lactate can comprise performing or having performed an assay on a sample obtained from the subject to determine/measure the level of lactate in the subject. In some embodiments of any of the aspects, the step of determining if the subject has a high level of lactate can comprise ordering or requesting an assay on a sample obtained from the subject to determine/measure the level of lactate in the subject. In some embodiments of any of the aspects, the step of determining if the subject has a high level of lactate can comprise receiving the results of an assay on a sample obtained from the subject to determine/measure the level of lactate in the subject. In some embodiments of any of the aspects, the step of determining if the subject has a high level of lactate can comprise receiving a report, results, or other means of identifying the subject as a subject with a high level of lactate.

In one aspect of any of the embodiments, described herein is a method of enhancing exercise endurance in a subject in need thereof, the method comprising: a) determining if the subject has a high level of lactate; and b) instructing or directing that the subject be administered Veillonella probiotic bacteria and or propionate if the level of lactate is high relative to a reference. In some embodiments of any of the aspects, the step of determining if the subject has a high level of lactate can comprise i) obtaining or having obtained a sample from the subject and ii) performing or having performed an assay on the sample obtained from the subject to determine/measure the level of lactate in the subject. In some embodiments of any of the aspects, the step of determining if the subject has a high level of lactate can comprise performing or having performed an assay on a sample obtained from the subject to determine/measure the level of lactate in the subject. In some embodiments of any of the aspects, the step of determining if the subject has a high level of lactate can comprise ordering or requesting an assay on a sample obtained from the subject to determine/measure the level of lactate in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In some embodiments of any of the aspects, the method comprises administering Veillonella probiotic bacteria and or propionate to a subject previously determined to have a level of propionate that is low relative to a reference. In some embodiments of any of the aspects, described herein is a method of enhancing exercise endurance in a subject in need thereof, the method comprising: a) first determining the level of propionate in a sample obtained from a subject; and b) then administering a Veillonella probiotic bacteria and or propionate to the subject if the level of propionate is low relative to a reference.

In some embodiments of any of the aspects, the method comprises administering Veillonella probiotic bacteria and or propionate to a subject previously determined to have a level of propionate that is low relative to a reference. In some embodiments of any of the aspects, the step of determining if the subject has a low level of propionate can comprise i) obtaining or having obtained a sample from the subject and ii) performing or having performed an assay on the sample obtained from the subject to determine/measure the level of propionate in the subject. In some embodiments of any of the aspects, the step of determining if the subject has a low level of propionate can comprise performing or having performed an assay on a sample obtained from the subject to determine/measure the level of propionate in the subject. In some embodiments of any of the aspects, the step of determining if the subject has a low level of propionate can comprise ordering or requesting an assay on a sample obtained from the subject to determine/measure the level of propionate in the subject. In some embodiments of any of the aspects, the step of determining if the subject has a low level of propionate can comprise receiving the results of an assay on a sample obtained from the subject to determine/measure the level of propionate in the subject. In some embodiments of any of the aspects, the step of determining if the subject has a low level of propionate can comprise receiving a report, results, or other means of identifying the subject as a subject with a low level of propionate.

In one aspect of any of the embodiments, described herein is a method of enhancing exercise endurance in a subject in need thereof, the method comprising: a) determining if the subject has a low level of propionate; and b) instructing or directing that the subject be administered Veillonella probiotic bacteria and or propionate if the level of propionate is low relative to a reference. In some embodiments of any of the aspects, the step of determining if the subject has a low level of propionate can comprise i) obtaining or having obtained a sample from the subject and ii) performing or having performed an assay on the sample obtained from the subject to determine/measure the level of propionate in the subject. In some embodiments of any of the aspects, the step of determining if the subject has a low level of propionate can comprise performing or having performed an assay on a sample obtained from the subject to determine/measure the level of propionate in the subject. In some embodiments of any of the aspects, the step of determining if the subject has a low level of propionate can comprise ordering or requesting an assay on a sample obtained from the subject to determine/measure the level of propionate in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

### Administration

The compositions and methods described herein can be administered to an athlete or a subject in need of enhanced exercise endurance. The compositions as described herein can be administered in the form of a food, beverage, dietary supplement. In some embodiments, the compositions can be formulated as a medical food, or in certain embodiments (e.g., with an acceptable carrier) are formulated for treatment of symptoms of a disease or disorder characterized by decreased exercise endurance.

In some embodiments of any of the aspects, enhanced exercise endurance comprises increased time spent on an exercise until exhaustion time. In some embodiments of any of the aspects, exercise can include running, rowing, or engaging in any sport or physical activity that can increase physical exertion. In some embodiments of any of the aspects, the methods described herein comprise administering an effective amount of compositions described herein, e.g. Veillonella probiotic bacteria and or propionate to a subject in order to reduce exercise-induced exhaustion or to enhance exercise endurance. As used herein, “reducing exercise-induced exhaustion” is ameliorating any condition or symptom associated with exercise. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, rectal, intracolonic, gastrointestinal, or parenteral administration. Administration can be local or systemic.

The term “effective amount” as used herein refers to the amount of Veillonella probiotic bacteria and or propionate needed to alleviate at least one or more symptom of the exercise-induced exhaustion, and relates to a sufficient amount of composition to provide the desired effect. The term “therapeutically effective amount” therefore refers to an amount of Veillonella probiotic bacteria and or propionate that is sufficient to provide a particular anti-exercise-induced exhaustion symptom or pro-exercise endurance effect when administered to a typical subject. An effective amount as used herein, in various contexts, would also include an amount sufficient to delay the development of a symptom of exercise-induced exhaustion, alter the course of a symptom of exercise-induced exhaustion (for example but not limited to, slowing the progression of a symptom of exercise-induced exhaustion), or reverse a symptom of exercise-induced exhaustion. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a concentration range that includes the IC50 (i.e., the concentration of propionate, which achieves a half-maximal inhibition of symptoms) as determined in cell culture, or in an appropriate animal model. Levels in the gastrointestinal tract can be measured, for example, by high performance liquid chromatography. The effects of any particular dosage can be monitored by a suitable bioassay, e.g., assay for propionate, among others. The dosage can be determined by a physician and adjusted, as necessary, to suit observed effects of the treatment.

In some embodiments, the technology described herein relates to a composition comprising Veillonella probiotic bacteria and or propionate as described herein, and optionally an acceptable carrier. In some embodiments, the active ingredients of the composition comprise Veillonella probiotic bacteria and or propionate as described herein. In some embodiments, the active ingredients of the composition consist essentially of Veillonella probiotic bacteria and or propionate as described herein. In some embodiments, the active ingredients of the composition consist of Veillonella probiotic bacteria and or propionate as described herein. Acceptable carriers and diluents include saline, aqueous buffer solutions, solvents and/or dispersion media. The use of such carriers and diluents is well known in the art. Some non-limiting examples of materials which can serve as acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in formulations. Wetting agents, coloring agents, release agents, coating agents, sweetening agents, flavoring agents, perfuming agents, preservative and antioxidants can also be present in the formulation. The terms such as “excipient”, “carrier”, “acceptable carrier” or the like are used interchangeably herein. In some embodiments, the carrier inhibits the degradation of the active agent, e.g. Veillonella probiotic bacteria and or propionate as described herein.

Compositions comprising Veillonella probiotic bacteria and or propionate can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the acceptable salt of the disclosed compounds (e.g., sodium propionate or any other acceptable salt of propionate), and may be prepared by methods of pharmacy well known to those skilled in the art. See generally, Remington: The Science and Practice of Pharmacy, 21st Ed., Lippincott, Williams, and Wilkins, Philadelphia Pa. (2005). In some embodiments, the pill or composition suitable for oral administration can comprise an enteric coating. In some embodiments, the pill or composition suitable for oral administration can exclude oxygen, to provide an anaerobic environment.

In some embodiments, the Veillonella probiotic bacteria is lyophilized. In some embodiments, the Veillonella probiotic bacteria is in a viable form, measurable by colony forming units per milliliter (CFU/mL). In some embodiments, the Veillonella probiotic bacteria is in a non-viable form, measurable by an equivalent viable dose in CFU/mL. In some embodiments, the Veillonella probiotic bacteria is administered at about 1.0*10⁸-1.0*10¹⁰ CFU/mL. In some embodiments, the Veillonella probiotic bacteria is administered at about 1.0*10⁸, about 1.1*10⁸, about 1.2*10⁸, about 1.3*10⁸, about 1.4*10⁸, about 1.5*10⁸, about 1.6*10⁸, about 1.7*10⁸, about 1.8*10⁸, about 1.9*10⁸, about 2.0*10⁸, about 2.1*10⁸, about 2.2*10⁸, about 2.3*10⁸, about 2.4*10⁸, about 2.5*10⁸, about 2.6*10⁸, about 2.7*10⁸, about 2.8*10⁸, about 2.9*10⁸, about 3.0*10⁸, about 3.1*10⁸, about 3.2*10⁸, about 3.3*10⁸, about 3.4*10⁸, about 3.5*10⁸, about 3.6*10⁸, about 3.7*10⁸, about 3.8*10⁸, about 3.9*10⁸, about 4.0*10⁸, about 4.1*10⁸, about 4.2*10⁸, about 4.3*10⁸, about 4.4*10⁸, about 4.5*10⁸, about 4.6*10⁸, about 4.7*10⁸, about 4.8*10⁸, about 4.9*10⁸, about 5.0*10⁸, about 5.1*10⁸, about 5.2*10⁸, about 5.3*10⁸, about 5.4*10⁸, about 5.5*10⁸, about 5.6*10⁸, about 5.7*10⁸, about 5.8*10⁸, about 5.9*10⁸, about 6.0*10⁸, about 6.1*10⁸, about 6.2*10⁸, about 6.3*10⁸, about 6.4*10⁸, about 6.5*10⁸, about 6.6*10⁸, about 6.7*10⁸, about 6.8*10⁸, about 6.9*10⁸, about 7.0*10⁸, about 7.1*10⁸, about 7.2*10⁸, about 7.3*10⁸, about 7.4*10⁸, about 7.5*10⁸, about 7.6*10⁸, about 7.7*10⁸, about 7.8*10⁸, about 7.9*10⁸, about 8.0*10⁸, about 8.1*10⁸, about 8.2*10⁸, about 8.3*10⁸, about 8.4*10⁸, about 8.5*10⁸, about 8.6*10⁸, about 8.7*10⁸, about 8.8*10⁸, about 8.9*10⁸, about 9.0*10⁸, about 9.1*10⁸, about 9.2*10⁸, about 9.3*10⁸, about 9.4*10⁸, about 9.5*10⁸, about 9.6*10⁸, about 9.7*10⁸, about 9.8*10⁸, about 9.9*10⁸, about 1.0*10⁹, about 1.1*10⁹, about 1.2*10⁹, about 1.3*10⁹, about 1.4*10⁹, about 1.5*10⁹, about 1.6*10⁹, about 1.7*10⁹, about 1.8*10⁹, about 1.9*10⁹, about 2.0*10⁹, about 2.1*10⁹, about 2.2*10⁹, about 2.3*10⁹, about 2.4*10⁹, about 2.5*10⁹, about 2.6*10⁹, about 2.7*10⁹, about 2.8*10⁹, about 2.9*10⁹, about 3.0*10⁹, about 3.1*10⁹, about 3.2*10⁹, about 3.3*10⁹, about 3.4*10⁹, about 3.5*10⁹, about 3.6*10⁹, about 3.7*10⁹, about 3.8*10⁹, about 3.9*10⁹, about 4.0*10⁹, about 4.1*10⁹, about 4.2*10⁹, about 4.3*10⁹, about 4.4*10⁹, about 4.5*10⁹, about 4.6*10⁹, about 4.7*10⁹, about 4.8*10⁹, about 4.9*10⁹, about 5.0*10⁹, about 5.1*10⁹, about 5.2*10⁹, about 5.3*10⁹, about 5.4*10⁹, about 5.5*10⁹, about 5.6*10⁹, about 5.7*10⁹, about 5.8*10⁹, about 5.9*10⁹, about 6.0*10⁹, about 6.1*10⁹, about 6.2*10⁹, about 6.3*10⁹, about 6.4*10⁹, about 6.5*10⁹, about 6.6*10⁹, about 6.7*10⁹, about 6.8*10⁹, about 6.9*10⁹, about 7.0*10⁹, about 7.1*10⁹, about 7.2*10⁹, about 7.3*10⁹, about 7.4*10⁹, about 7.5*10⁹, about 7.6*10⁹, about 7.7*10⁹, about 7.8*10⁹, about 7.9*10⁹, about 8.0*10⁹, about 8.1*10⁹, about 8.2*10⁹, about 8.3*10⁹, about 8.4*10⁹, about 8.5*10⁹, about 8.6*10⁹, about 8.7*10⁹, about 8.8*10⁹, about 8.9*10⁹, about 9.0*10⁹, about 9.1*10⁹, about 9.2*10⁹, about 9.3*10⁹, about 9.4*10⁹, about 9.5*10⁹, about 9.6*10⁹, about 9.7*10⁹, about 9.8*10⁹, about 9.9*10⁹, or about 1.0*10¹⁰ CFU/mL.

In some embodiments, the Veillonella probiotic bacteria can be at least one of Veillonella atypica, Veillonella dispar, or Veillonella parvula. In some embodiments, the Veillonella probiotic bacteria comprises Veillonella atypica. In some embodiments, the Veillonella probiotic bacteria comprises Veillonella dispar. In some embodiments, the Veillonella probiotic bacteria comprises Veillonella parvula. In some embodiments, the Veillonella probiotic bacteria comprises Veillonella atypica and Veillonella dispar. In some embodiments, the Veillonella probiotic bacteria comprises Veillonella atypica and Veillonella parvula. In some embodiments, the Veillonella probiotic bacteria comprises Veillonella dispar and Veillonella parvula. In some embodiments, the Veillonella probiotic bacteria comprises Veillonella atypica, Veillonella dispar, and Veillonella parvula. In some embodiments, the Veillonella probiotic bacteria is administered with an effective dose of propionate.

In some embodiments of any of the aspects, the Veillonella probiotic bacteria can be comprised by a consortium. As used herein, the terms “Consortia” or “Consortium” refer to combinations or a combination of selected microbes (e.g., a combination of different species or different strains of probiotic bacteria) resulting in increased lactate metabolism, increased production of SCFAs, and/or increased enhancement of exercise endurance when administered to a subject. In some embodiments, a consortium can provide enhanced increases in lactate metabolism, SCFA production, and/or exercise endurance in comparison to those obtained with a single microbe species or strain. In some embodiments of any of the aspects, a consortium comprises two or more types of microbes that can cooperate (i.e., cross-feed; see e.g., Falony G., ET al. 2006 Appl. Environ. Microbiol. 72(12): 7835-7841) to metabolize lactate and/or produce SCFAs. In some embodiments, a consortium comprises at least one type of microbe that is capable of metabolizing lactate into an intermediate product (e.g., pyruvate, acetyl-CoA, succinate, succinyl-CoA, methylmalonyl-CoA, propionyl-CoA; see e.g., FIG. 3A) that can be converted by a second type of microbe to an SCFA (e.g., propionate, acetate). In some embodiments, the first type of microbe expresses enzymes from the beginning of the methylmalonyl-CoA pathway, and the second type of microbe expresses enzymes from the end of the methylmalonyl-CoA pathway (e.g., the methylmalonyl-CoA pathway in order comprises: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase).

It is contemplated that two or more, three or more, four or more, five or more, 6 or more, 7 or more, 8 or more, 9 or more, 10 or more, 11 or more, 12 or more, or even 13 or more different microbes can form a consortium that promotes increased lactate metabolism and thereby increases SCFA production and exercise endurance. One benefit of the use of a consortium is that the expression or activity of the rate-limiting enzyme of the pathway can be compensated for by providing for greater amounts of the enzyme to be made. Applying this principle to the next slowest enzyme-catalyzed reaction in an iterative fashion can provide further enhancement in lactate metabolism, SCFA production, and/or exercise endurance for a consortium, relative to a single microbe.

In some embodiments, the Veillonella probiotic bacteria and or propionate can be administered in the form of a food, a beverage, or a dietary supplement (see e.g., WO 2012/177556 A2; U.S. Pat. Nos. 9,693,578 B2; 6,468,525 B1; incorporated herein by reference in their entireties). In some embodiments, the food can comprise a medical food, a dairy product (e.g., milk, yogurt, curd, cheese or an infant formula), a cereal product (e.g., rice, wheat, oats, barley, corn, rye, sorghum, millet, or triticale), a fermented food product, a dried food product, or a rehydrated food product. The food product can also be a vegetable or a fruit product, for example, a juice, a puree, a concentrate, a paste, a sauce, a pickle or a ketchup. Exemplary vegetables and fruits include, without limitation, squashes, e.g., zucchini, yellow squash, winter squash, pumpkin; potatoes, asparagus, broccoli, Brussels sprouts, beans, e.g., green beans, wax beans, lima beans, fava beans, soy beans, cabbage, carrots, cauliflower, cucumbers, kohlrabi, leeks, scallions, onions, sugar peas, English peas, peppers, turnips, rutabagas, tomatoes, apples, pears, peaches, plums, strawberries, raspberries, blackberries, blueberries, lingonberries, boysenberries, gooseberries, grapes, currants, oranges, lemons, grapefruit, bananas, mangos, kiwi fruit, and carambola. The food product can also be a “milk” made from grains (barley, oat or spelt “milk”) tree nuts (almond, cashew, coconut, hazelnut or walnut “milk”), legumes (soy, peanut, pea or lupin “milk”) or seeds (quinoa, sesame seed or sunflower seed “milk”). Also contemplated are food products comprising animal proteins, for example, meat, for example, sausages, dried meats, fish and dried fish products.

In some embodiments, the beverage can comprise any liquid food product formulated from a food product referred to herein. Non-limiting beverage examples comprise a dairy beverage, a fruit beverage, a fruit juice, a fruit drink (e.g., 0 to 29% fruit juice), a fruit nectar (e.g., 30 to 99% fruit juice), or a vegetable beverage. In some embodiments, the Veillonella probiotic bacteria and or propionate can be administered in the form of a dietary supplement. Non-limiting examples of dietary supplements comprise vitamins, minerals, amino acids, herbs, and or botanicals in combination with the Veillonella probiotic bacteria and or propionate. Dietary supplements are often administered in the form of an ingestible pill.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the Veillonella probiotic bacteria and or propionate can be administered in a sustained release formulation.

Controlled-release products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the composition comprising at least one probiotic bacteria as described herein further comprises an enteric coating or similar composition to promote survival of or avoid the acidity of the stomach and permit delivery into the small or large intestines. Non-limiting examples of enteric coatings include cellulose acetate phthalate (CAP), hydroxypropyl methylcellulose phthalate (HPMCP), hydroxypropyl methylcellulose acetate succinate (HPMCAS), polyvinyl acetate phthalate, cellulose acetate trimellitate, shellac, polymethacrylic acid, polymethyl methacrylate, polyethyl methacrylate, polyethyl acrylate, hydroxypropyl methylcellulose, hydroxypropyl cellulose, polyvinylpyrrolidone, polyethylene glycol, polyvinyl alcohol, and mixtures thereof. In some embodiments, the enteric coating is pH sensitive. As a non-limiting example, the enteric coating dissolves at a pH greater than about 6.5-7, so as to prevent the release in the stomach and permit the release in the intestines. See e.g., US Patent Application 20190046457 and U.S. Pat. No. 9,486,487, the contents of each of which are incorporated herein by reference in their entireties.

In some embodiments, the Veillonella probiotic bacteria described herein is administered as a single treatment, e.g., another treatment to enhance exercise endurance is not administered to the subject. In some embodiments, the propionate described herein is administered as a single treatment, e.g., another treatment to enhance exercise endurance is not administered to the subject. In some embodiments, the methods described herein can further comprise administering a second agent and/or treatment to the subject, e.g. as part of a combinatorial therapy. Non-limiting examples of a second agent and/or treatment can include an antibiotic, a probiotic, a prebiotic, a synbiotic, and/or a postbiotic.

In certain embodiments, an effective dose of a composition comprising the Veillonella probiotic bacteria and or propionate as described herein can be administered to a patient once. In certain embodiments, an effective dose of a composition comprising the Veillonella probiotic bacteria and or propionate can be administered to a patient repeatedly. For systemic administration, subjects can be administered a therapeutic amount of a composition comprising the Veillonella probiotic bacteria and or propionate, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. lactate by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the Veillonella probiotic bacteria and or propionate. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising the Veillonella probiotic bacteria and or propionate can be administered over a period of time, such as over a 5 minute, 10 minute, 15 minute, 20 minute, or 25 minute period.

The dosage ranges for the administration of the Veillonella probiotic bacteria and or propionate, according to the methods described herein depend upon, for example, the form of the Veillonella probiotic bacteria and or propionate, its potency, and the extent to which symptoms, markers, or indicators of a condition described herein are desired to be reduced, for example the percentage reduction desired for lactate or the extent to which, for example, increased propionate are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of the Veillonella probiotic bacteria and or propionate in, e.g. the treatment of a condition described herein, or to induce a response as described herein (e.g. decreased lactate, increased propionate) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. lactate, propionate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. increased exercise endurance, increased length of time for exercise before exhaustion). It is well within the ability of one skilled in the art to monitor efficacy of administration and/or treatment by measuring any one of such parameters, or any combination of parameters. Efficacy can be assessed in animal models of a condition described herein, for example enhancement of exercise endurance. When using an experimental animal model, efficacy of treatment is evidenced when a statistically significant change in a marker is observed, e.g. lactate and or propionate levels.

In vitro and animal model assays are provided herein which allow the assessment of a given dose of the Veillonella probiotic bacteria and or propionate. By way of non-limiting example, the effects of a dose of the Veillonella probiotic bacteria and or propionate can be assessed by in an animal model, e.g. treadmill run time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10%-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of exercise. A subject can be male or female.

A subject can be one who has been previously diagnosed with or identified as suffering from or having a condition in need of treatment (e.g. reduced exercise endurance) or one or more complications related to such a condition, and optionally, have already undergone treatment for reduced exercise endurance or the one or more complications related to exercise. Alternatively, a subject can also be one who has not been previously diagnosed as having reduced exercise endurance or one or more complications related to exercise. For example, a subject can be one who exhibits one or more risk factors for reduced exercise endurance or one or more complications related to exercise or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant amino acid or DNA sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as descried herein. Accordingly, the percentage of “sequence similarity” is the percentage of amino acids which is either identical or conservatively changed; e.g., “sequence similarity”=(% sequence identity)+(% conservative changes). The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology (see also e.g., U.S. Pat. Nos 5,654,418; 6,344,316; and 8,889,358; and US Patent Application Numbers US 2013/0157265 and US 2018/0195111, which are incorporated by reference in their entireties).

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having undergone exercise, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the Veillonella probiotic bacteria and or propionate described herein is exogenous. In some embodiments of any of the aspects, the Veillonella probiotic bacteria and or propionate described herein is ectopic. In some embodiments of any of the aspects, the Veillonella probiotic bacteria and or propionate described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. reduced exercise endurance. The term “treating” includes reducing or alleviating at least one adverse effect or symptom of a condition, disease or disorder associated with exercise. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A composition comprising at least one Veillonella probiotic bacterium and an acceptable excipient, diluent, or carrier.
2. The composition of paragraph 1, wherein the Veillonella probiotic bacterium is selected from the group consisting of Veillonella atypica, Veillonella dispar, and Veillonella parvula.
3. The composition of any one of paragraphs 1-2, wherein the Veillonella probiotic bacterium comprises Veillonella atypica, Veillonella dispar, and Veillonella parvula.
4. The composition of any one of paragraphs 1-3, wherein the Veillonella probiotic bacterium comprises and expresses genes encoding enzymes sufficient to metabolize the conversion of lactate into short-chain fatty acids comprising one or more of propionate and acetate.
5. The composition of any one of paragraphs 1-4, wherein the Veillonella probiotic bacterium comprises and expresses genes encoding enzymes from the methylmalonyl-CoA pathway.
6. The composition of any one of paragraphs 1-5, wherein the Veillonella probiotic bacterium is viable and lyophilized.
7. The composition of any one of paragraphs 1-6, wherein the acceptable excipient, diluent, or carrier comprises water, saline, dextrose, glycerol, ethanol or the like and combinations thereof.
8. The composition of any one of paragraphs 1-7, wherein the composition is in the form of a pill, a tablet, or a capsule.
9. A method of enhancing exercise endurance, comprising administering an effective dose of the composition of any of paragraphs 1-8 to a subject in need thereof.
10. The method of paragraph 9, wherein the composition is administered via an oral, enteric, gastrointestinal, rectal, or parenteral route.
11. The method of any one of paragraphs 9-10, wherein the subject is a human athlete or human in need of enhanced exercise endurance.
12. The method of any one of paragraphs 9-11, wherein enhanced exercise endurance comprises increased time spent on an exercise until exhaustion time.
13. A device preloaded for administration to a body cavity comprising an effective dose of propionate to enhance exercise endurance.
14. The device of paragraph 13, wherein the propionate comprises 10 mM-1000 mM sodium propionate.
15. The device of any one of paragraphs 13-14, comprising a suppository.
16. The device of any one of paragraphs 13-15, wherein the body cavity is the rectum.
17. A method of enhancing exercise endurance comprising administering to a subject in need thereof an effective amount of propionate.
18. The method of paragraph 17, wherein the propionate comprises 10 mM-1000 mM sodium propionate.
19. The method of any one of paragraphs 17-18, wherein the propionate is administered via a rectal, intracolonic, gastrointestinal, enteric, oral, or parenteral route.
20. The method of any one of paragraphs 17-19, wherein the subject is a human athlete or human in need of enhanced exercise endurance.
21. An engineered probiotic bacterium that is effective at enhancing exercise endurance.
22. The engineered probiotic bacterium of paragraph 21, wherein the engineered probiotic bacterium comprises and expresses genes encoding enzymes sufficient to metabolize the conversion of lactate into short-chain fatty acids comprising one or more of propionate and acetate.
23. The engineered probiotic bacterium of any one of paragraphs 21-22, wherein the engineered probiotic bacterium comprises and expresses genes encoding enzymes from the methylmalonyl-CoA pathway.
24. The engineered probiotic bacterium of any one of paragraphs 21-23, wherein the engineered probiotic bacterium comprises and expresses genes encoding enzymes selected from the group consisting of: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.
25. The engineered probiotic bacterium of any one of paragraphs 21-24, wherein enhanced exercise endurance comprises increased time spent on an exercise until exhaustion time.
26. A food, beverage, or dietary supplement composition comprising a bacterium that comprises and expresses genes encoding enzymes sufficient to metabolize the conversion of lactate into short-chain fatty acids comprising one or more of propionate and acetate.
27. The food, beverage, or dietary supplement composition of paragraph 26, wherein the bacterium is present in an amount sufficient to increase exercise endurance in a subject consuming the subject.
28. The food, beverage, or dietary supplement composition of any one of paragraphs 26-27, wherein the bacterium is selected from the group consisting of Veillonella atypica, Veillonella dispar, and Veillonella parvula.
29. The food, beverage, or dietary supplement composition of any one of paragraphs 26-28, wherein the bacterium comprises and expresses genes encoding enzymes from the methylmalonyl-CoA pathway.
30. The food, beverage, or dietary supplement composition of any one of paragraphs 26-29, wherein the bacterium comprises and expresses genes encoding enzymes selected from the group consisting of: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.
31. The composition of any one of paragraphs 1-8, for use in enhancing exercise endurance.
32. The device of any one of paragraphs 13-16, for use in enhancing exercise endurance.
33. The engineered probiotic bacterium of any one of paragraphs 21-25, for use in enhancing exercise endurance.
34. The food, beverage, or dietary supplement composition of any one of paragraphs 26-30, for use in enhancing exercise endurance.
35. Use of the composition of any one of paragraphs 1-8, for enhancing exercise endurance.
36. Use of the device of any one of paragraphs 13-16, for enhancing exercise endurance.
37. Use of the engineered probiotic bacterium of any one of paragraphs 21-25, for enhancing exercise endurance.
38. Use of the food, beverage, or dietary supplement composition of any one of paragraphs 26-30, for enhancing exercise endurance.

### EXAMPLES

### Example 1

Meta'omic Analysis of Elite Athletes Identifies a Performance-Enhancing Microbe that Functions Via Lactate Metabolism.

The human gut microbiome encodes a vast metabolic repertoire with direct impacts on many aspects of host physiology, yet it is unknown whether it has any bearing on physical performance or exercise. To begin to address this question, a longitudinal metagenomic analysis was performed on marathon runners to identify microbiome features associated with intense physical activity. The strongest microbiome feature enriched post-marathon was an increase in the abundance of the bacterial genus Veillonella. Veillonella atypica was isolated directly from the marathon runners, and it was found that upon inoculation in laboratory mice in an AB/BA crossover study testing treadmill runtime to exhaustion, runtime was increased 13% in a V. atypica-dependent manner. V. atypica has a preference for lactate as its primary carbon source, and using shotgun metagenomic analysis in a cohort of elite athletes it was found that every differentially expressed gene family in the pathway metabolizing lactate to the short-chain fatty acid propionate was at higher relative abundance post-exercise. Using ¹³C₃-labeled lactate in mice, it was demonstrated that serum lactate crosses the epithelial barrier into the lumen of the gut. Intrarectal instillation of propionate was sufficient to reproduce the increased treadmill runtime performance observed with V. atypica gavage. V. atypica improves runtime via its metabolic conversion of exercise-induced lactate into propionate, thereby identifying a natural, microbiome-encoded enzymatic process that enhances athletic performance.

Longitudinal stool samples were collected from elite athletes to determine whether the metabolic potential of the human gut microbiome has a direct role in physical performance. The bacterial genus Veillonella was identified as being the strongest microbiome feature associated with running a marathon. An athlete-derived strain of Veillonella atypica significantly enhanced runtime to exhaustion in mice by a mechanism involving the fermentation of muscle-derived lactate into propionate, which is in itself is sufficient to reproduce the performance benefit of whole V. atypica.

Gut Veillonella Abundance is Significantly Associated with Marathon Running

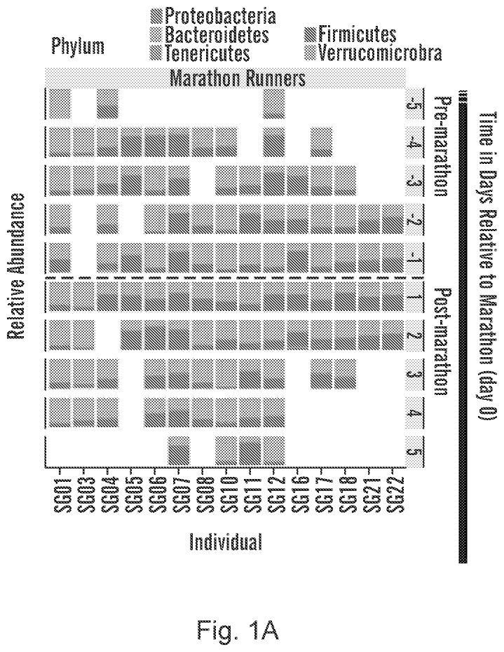

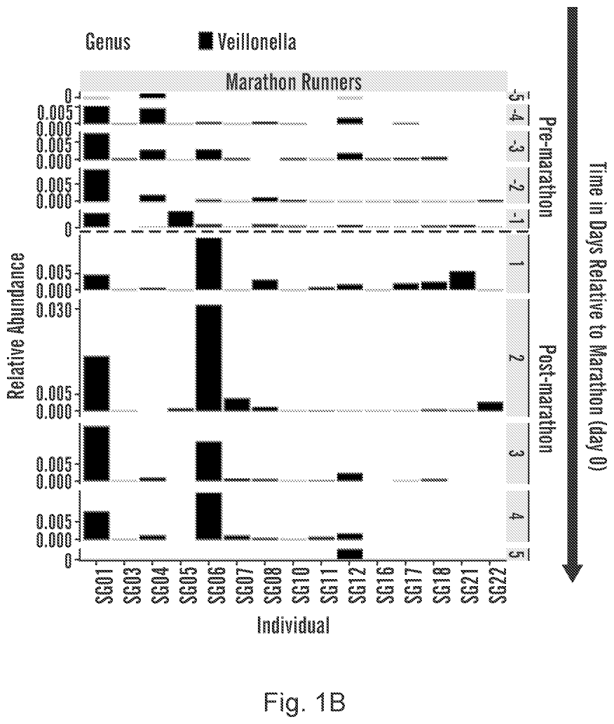

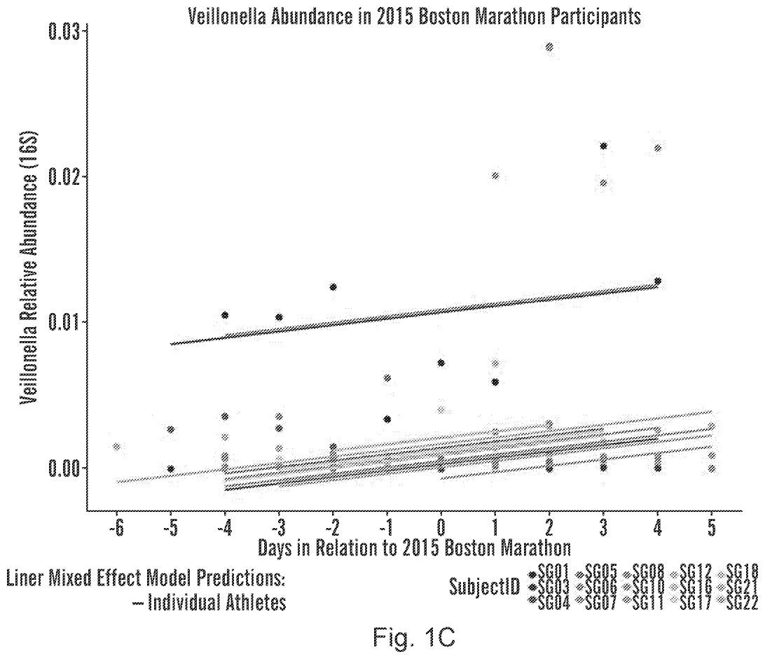

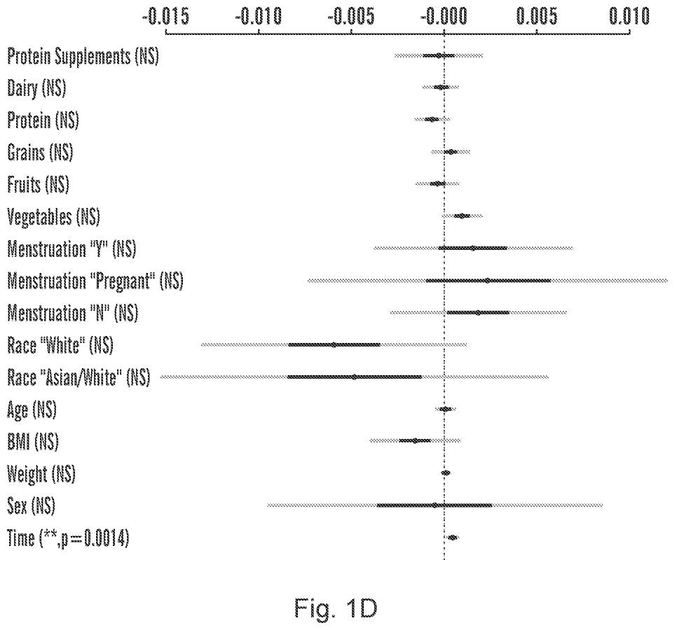

To identify gut bacteria associated with athletic performance and recovery states, recruitment was performed for athletes who had run a marathon (n=15) along with a set of sedentary controls (n=10) and conducted 16S rDNA sequencing on approximately daily samples collected up to one week before and one week after marathon day (n=209 samples). Phylum-level relative abundance partitioned by individual, time (−5 to +5 days in relation to running the Marathon), and whether the participant was an athlete (see e.g., FIG. 1A) showed that, at this high-level taxonomic view, any orthogonal differences were likely due to variation at the level of the individual. The bacterial genus Veillonella was the most differentially abundant microbiome feature between pre- and post-exercise states. Veillonella had a significant difference in relative abundance (P=0.02; Wilcoxon rank sum test with continuity correction) between samples collected before and after exercise (see e.g., FIG. 1B).

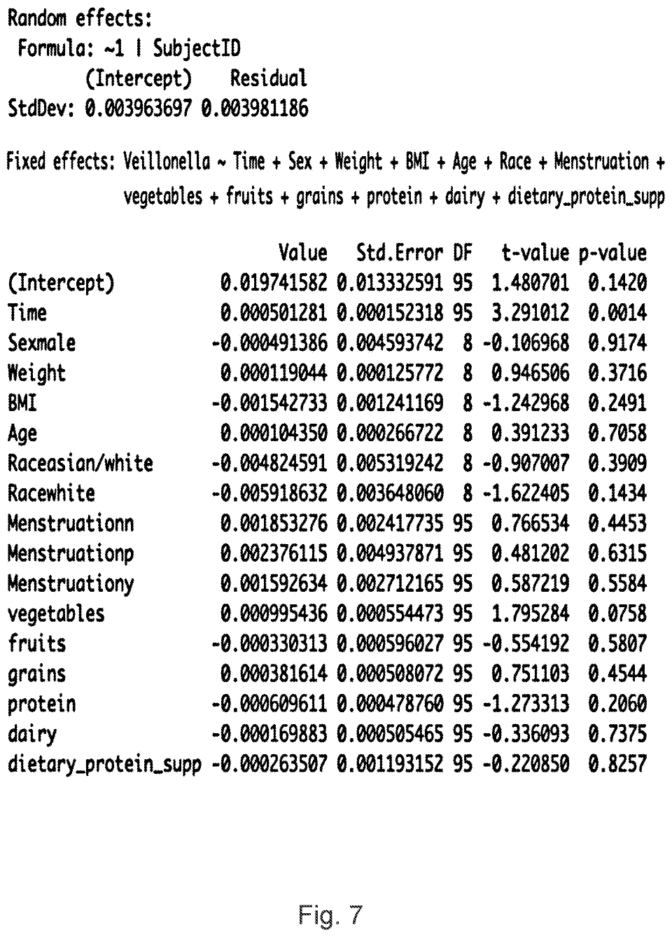

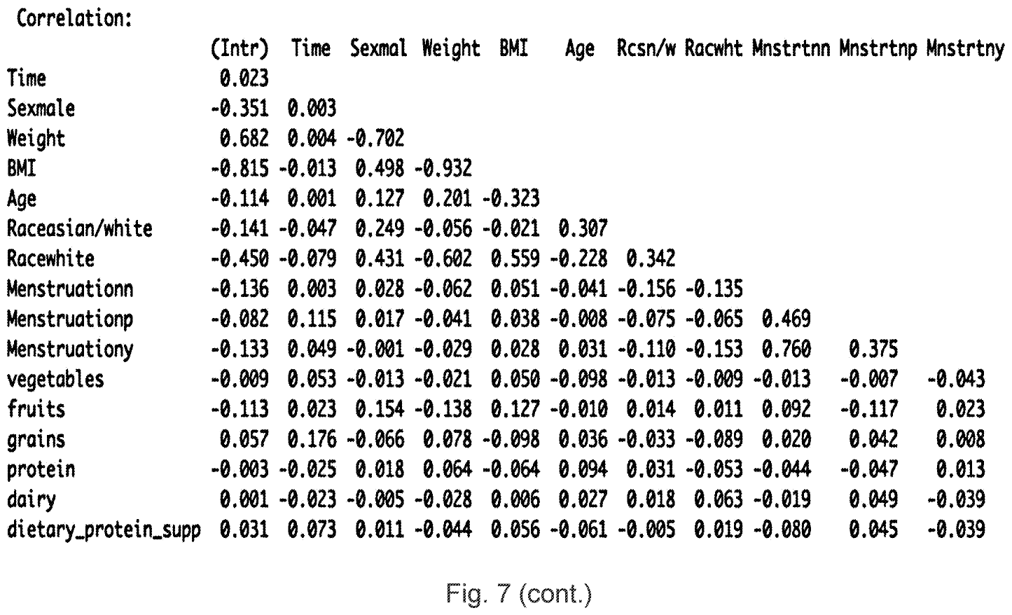

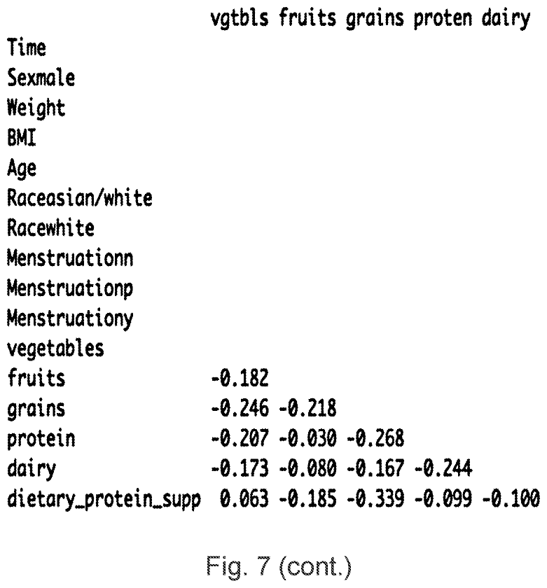

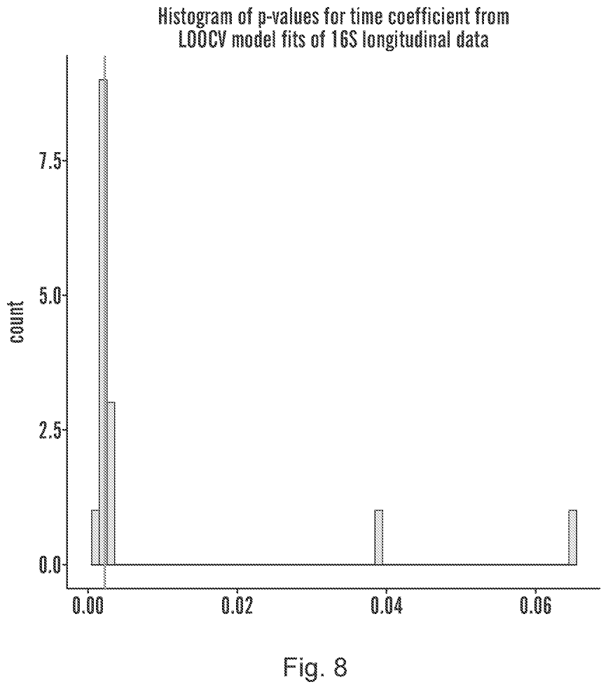

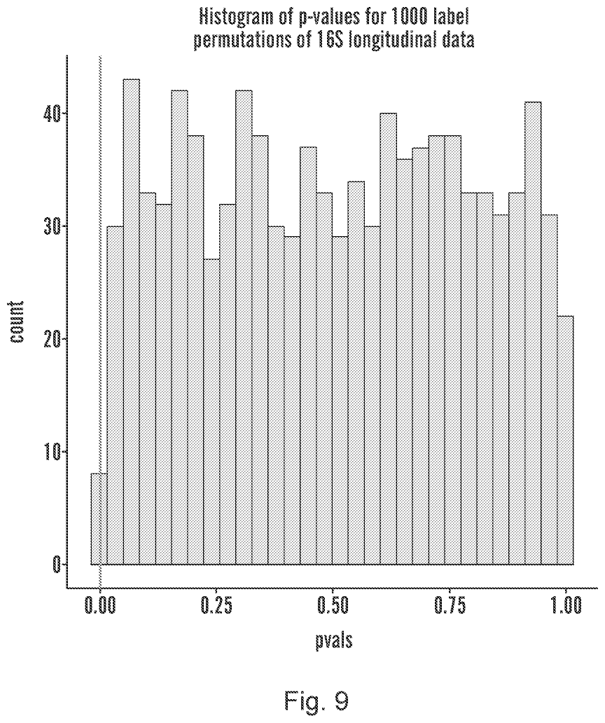

However, the longitudinal nature of the microbiome sampling coupled with the unique lifestyles of athletes means that diet, physical characteristics, age, gender, ethnicity, and the menstrual cycle could potentially confound the association between post-marathon state and Veillonella relative abundance. As some food compounds can selectively increase relative abundance of Veillonella, 1,267 meal records logging every instance of food consumption over the course of the study were quantified according to USDA MyPlate™ and associated with daily microbiome samples. To validate the significance of the association between Veillonella and post-marathon state, a series of generalized linear mixed effect models (GLMMs) were constructed to predict Veillonella relative abundance in the marathon participants (see e.g., FIG. 1C, methods) from both random effects (individual variation per athlete that manifests longitudinally) and fixed effects (USDA MyPlate™ consumption categories, protein powder supplementation, menstruation status, race, time, BMI, weight, gender, and age). Subsequently, significance was calculated for all coefficients included in the GLMM (see e.g., FIG. 1D and FIG. 7, Wald Z-tests), revealing no coefficients were significant except time in relation to the marathon in days (P=0.0014, Wald Z-test, n=15). Leave-one-out cross-validation (LOOCV) was performed for the GLMM analysis where the p-value for the time coefficient was calculated for all permutations of eliminating one athlete, which revealed a general trend of no individual athlete driving significance, with one minor outlier (see e.g., FIG. 8, Wald Z-tests). To ensure that an arbitrary shuffling of participant labeling would not yield significant results, the GLMM was trained 1000 times on input data with permuted labels, which generated uniformly distributed p-values and showed the significance of the original labeling (see e.g., FIG. 9, Wald Z-tests). Thus, the observed significance of the association between Veillonella relative abundance and pre- and post-marathon state was not confounded by any fixed effects. Additionally, Veillonella was more prevalent among runners than non-runners (see e.g., FIG. 1A-Fig. 1B and FIG. 9A-Fig. 9B), though this is not statistically significant. Though these correlations in themselves do not provide information as to whether Veillonella is functionally involved in the runners' performance, they do suggest it. To test whether Veillonella had any phenotypic impact on running ability, Veillonella was introduced to mice in a treadmill experiment.

### Veillonella Atypica Gavage Improves Treadmill Runtime in Mice.

To assess potential benefits of Veillonella on performance in an animal exercise model, an AB/BA crossover mouse experiment was designed spanning two weeks consisting of a control group (Lactobacillus bulgaricus gavage, n=16) and a treatment group (Veillonella gavage, n=16) with a treatment/control crossover happening between weeks (n=32 total mice). Lactobacillus bulgaricus was chosen as a control due its inability to catabolize lactate, mimicking bacterial load without impacting lactate metabolism. The Veillonella strain used, Veillonella atypica, was directly isolated from one of the marathon runners. Gastrointestinal (GI) passage time was estimated by quantitative polymerase chain reaction (qPCR)-based detection in the stool five hours post-gavage. Mice were administered either Veillonella atypica or Lactobacillus bulgaricus, then after five hours the mice were run until exhaustion on an electronic treadmill surrounded by a rest platform with a 15-degree incline starting at five meters/minute and increasing speed by one meter/minute every minute thereafter. Exhaustion was defined as a mouse failing to return to the treadmill from the rest platform (which carried a mild electrical current) after three consecutive attempts to continue running

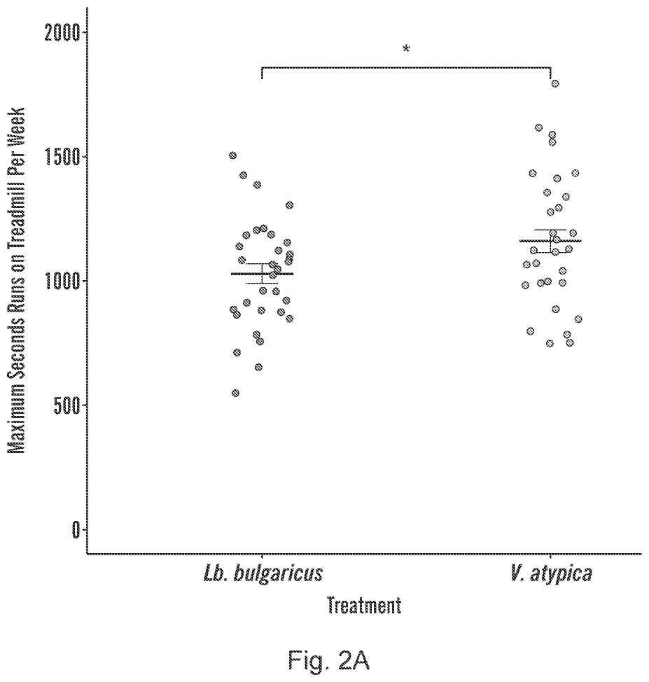

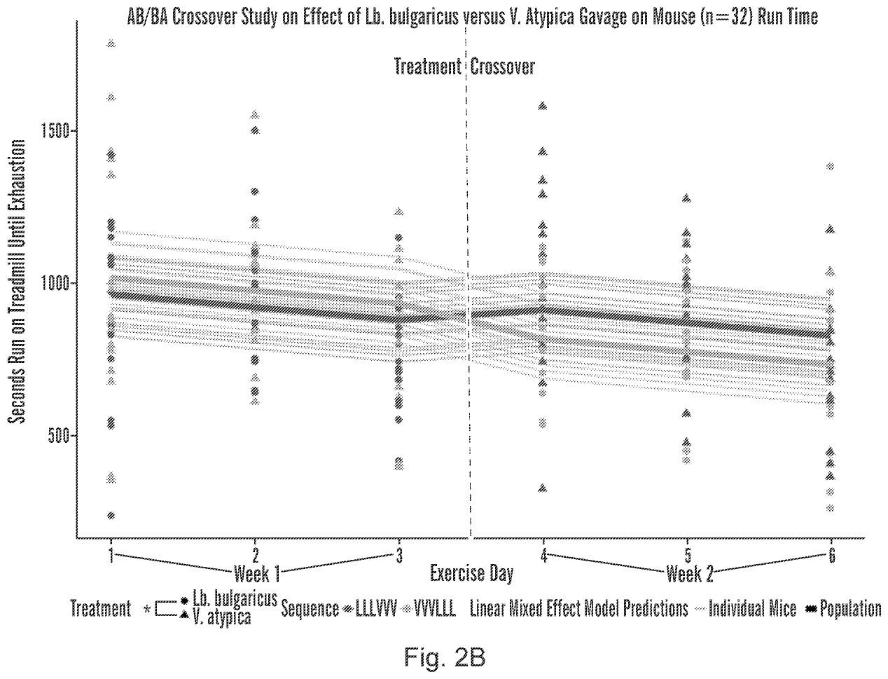

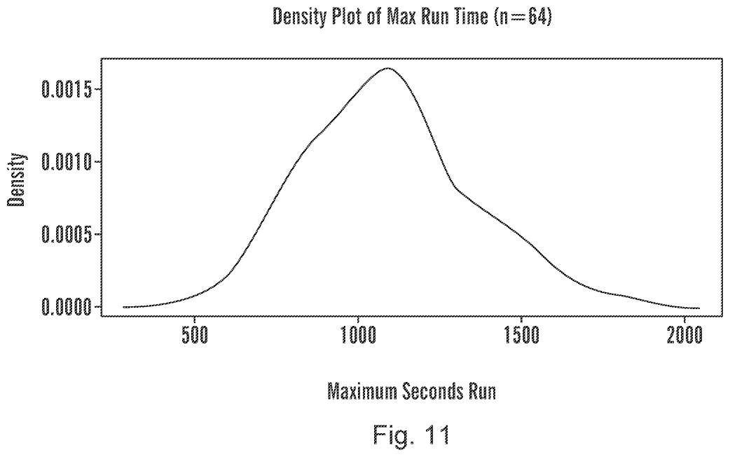

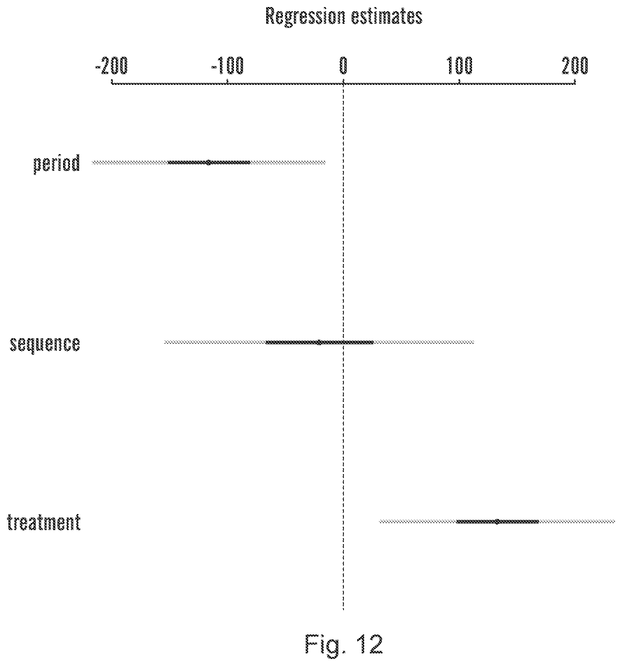

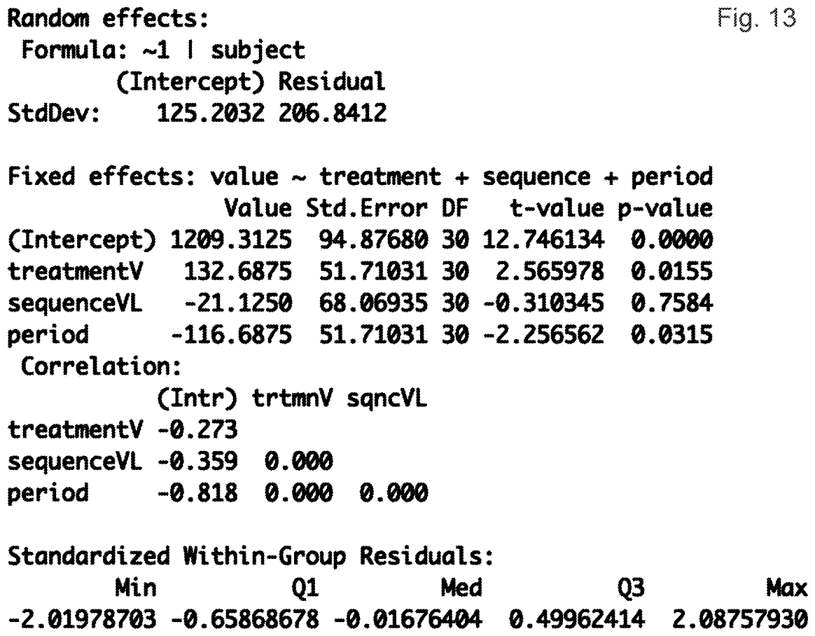

In aggregate, on both sides of the crossover, mice gavaged with Veillonella atypica had statistically significant longer maximum runtimes than mice gavaged with Lactobacillus bulgaricus (P=0.02 paired t-test, P=0.67 Shapiro-Wilk's normality test (valid to use t-test), see e.g., FIG. 2A, FIG. 11). Mice treated with Veillonella atypica ran on average 13% longer than the control group (see e.g., FIG. 2A). Comparisons of raw runtime in this context could be confounded both by carryover effect (modeled as a sequence effect) inherent in the longitudinal study design as well as unavoidably high inter-mouse variation. To account for these potential confounding variables, a series of GLMMs predicting runtime were constructed (see e.g., Methods). These models incorporate both random effects (individual variation per mouse that manifests longitudinally) and fixed effects (treatment day, treatment sequence, and treatment given). Visualization of all longitudinal data points with the GLMM predictions overlaid show both the effect of Veillonella atypica increasing performance on both sides of the crossover when aggregated by treatment group (thick lines) as well as the trends for each of the 32 individual mice (thin lines) (see e.g., FIG. 2B). Testing the significance of coefficients in the GLMM for their contribution to treadmill runtime (Wald-Z test) showed that sequence effect was not significant (P=0.758) while treatment day (P=0.031, negative effect on runtime) and Veillonella treatment (P=0.016, positive effect on runtime) were significant (see e.g., FIG. 12 and FIG. 13).

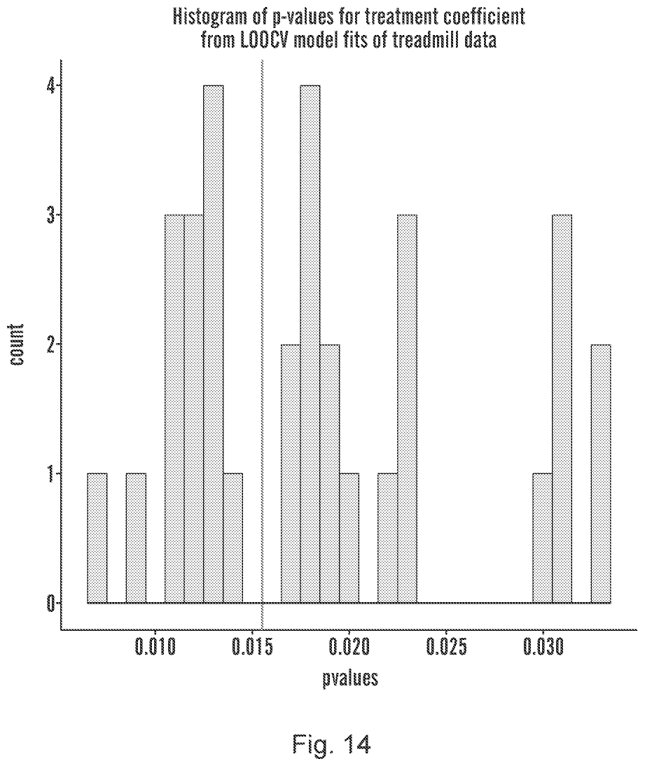

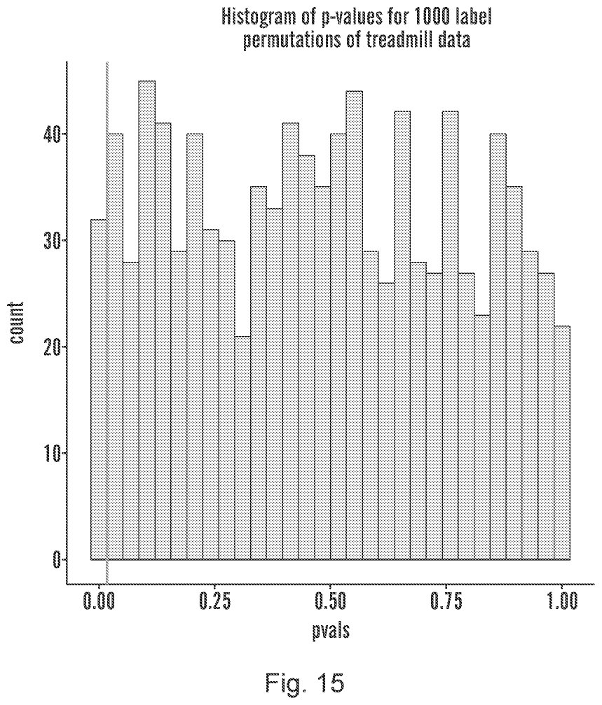

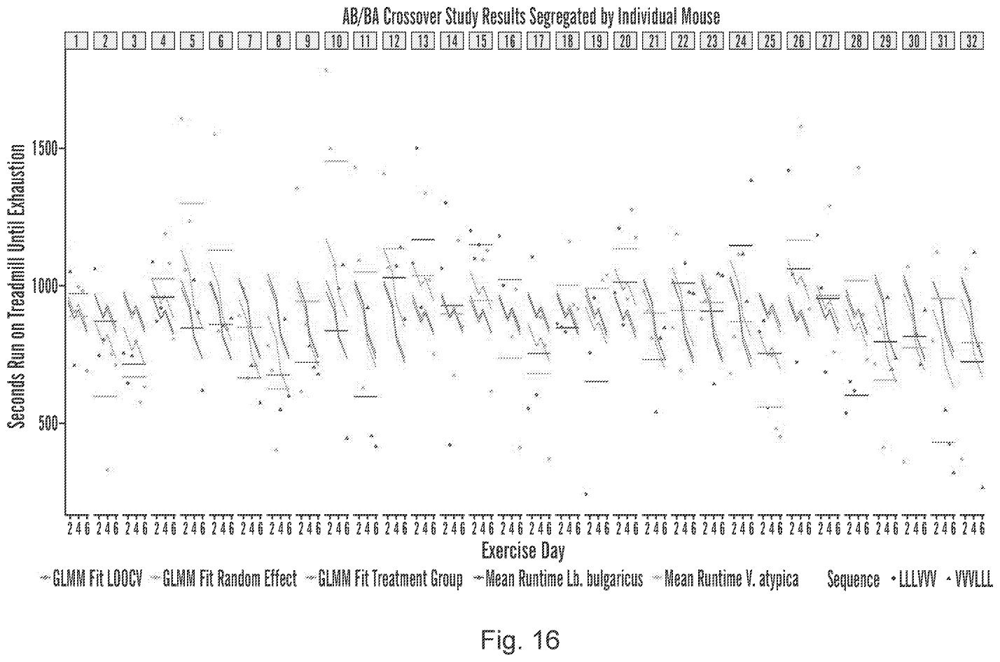

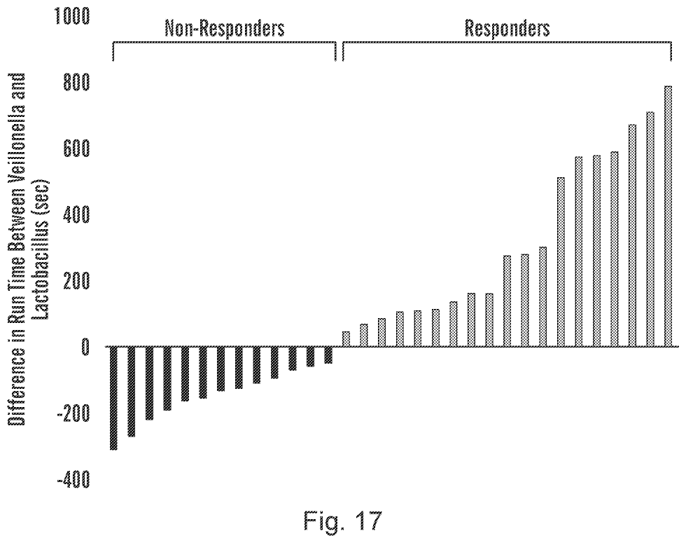

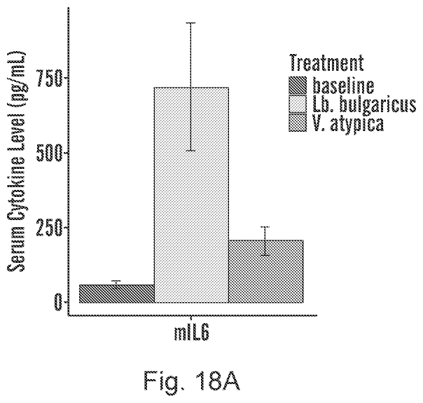

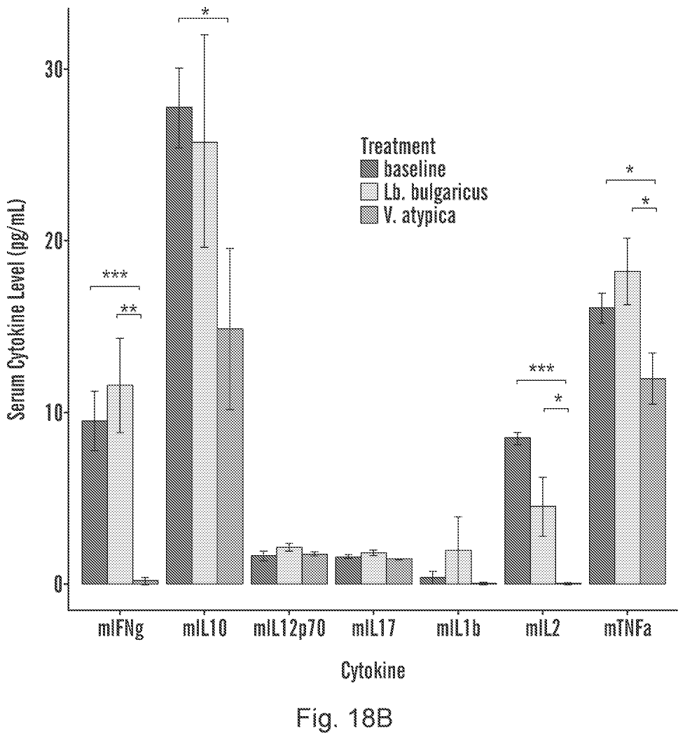

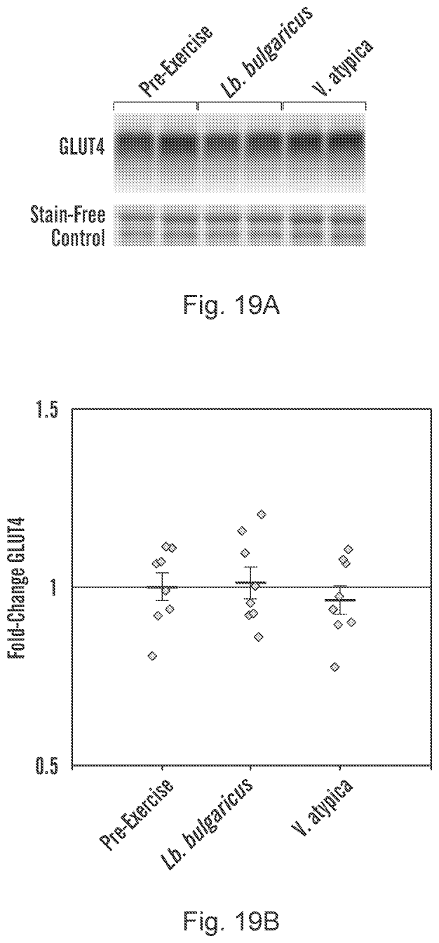

LOOCV was performed for the GLMM analysis where the p-value for the V. atypica treatment coefficient was calculated for all permutations of eliminating one mouse, which revealed that no individual mice were driving significance (see e.g., FIG. 14, Wald Z-tests). To ensure that an arbitrary shuffling of mouse labeling would not yield significant results, the GLMM was trained 1000 times on input data with permuted labels, which generated uniformly distributed p-values and showed the significance of the original labeling (see e.g., FIG. 15, Wald Z-tests). Per-mouse both run times were overlaid on the GLMM fits (see e.g., FIG. 16). The difference between max run time in Lb. Bulgaricus versus V. atypica gavage were segregated into “responders” and “non-responders” (see e.g., FIG. 17). This longitudinal modeling approach allows the interpretation that as the treadmill runs were conducted back-to-back each week on subsequent days, the mice in aggregate had decreasing runtimes as time to exhaustion decreases (visible as slope of predictions, see e.g., FIG. 2B), while Veillonella atypica treatment independently increased runtime (visible as crossover of predictions showing Veillonella treatment group having longer time to exhaustion on both sides of the crossover; see e.g., FIG. 2B). To identify possible biological mechanisms of Veillonella effect, the levels of various inflammatory cytokines in the blood were quantified immediately following treadmill run. Several pro-inflammatory cytokines, including TNFα and IFNγ, were significantly reduced in V. atypica-treated mice compared to both baseline and control treatment (see e.g., FIG. 18A and FIG. 18B). In a separate experiment, levels of the muscle glucose transporter GLUT4 was quantified to assess effects on muscle physiology, but no difference was found between V. atypica-treatment and control (see e.g., FIG. 19A and FIG. 19B). Altogether, taking into account inter-mouse variation, longitudinal study design, and possible carryover effect in an AB/BA crossover trial, Veillonella atypica treatment caused substantial increases in treadmill runtime in mice.

### The Athlete Gut Microbiome is Functionally Enriched for the Metabolism of Lactate to Propionate Post-Exercise.

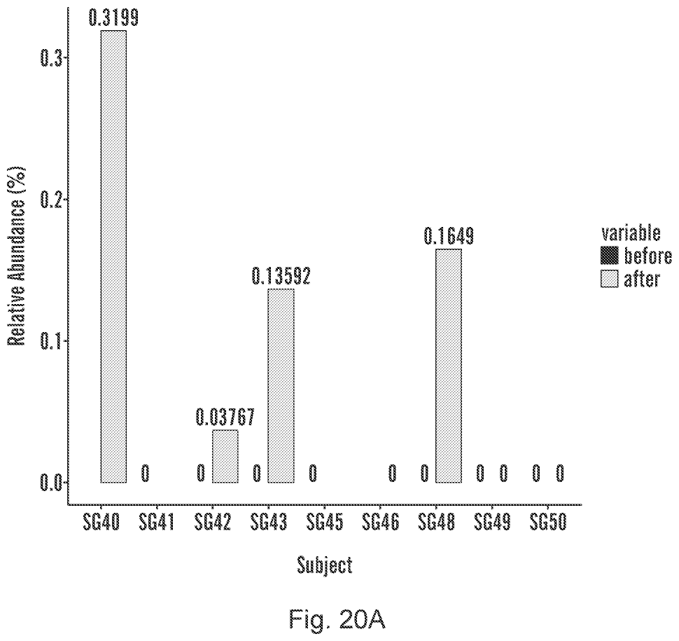

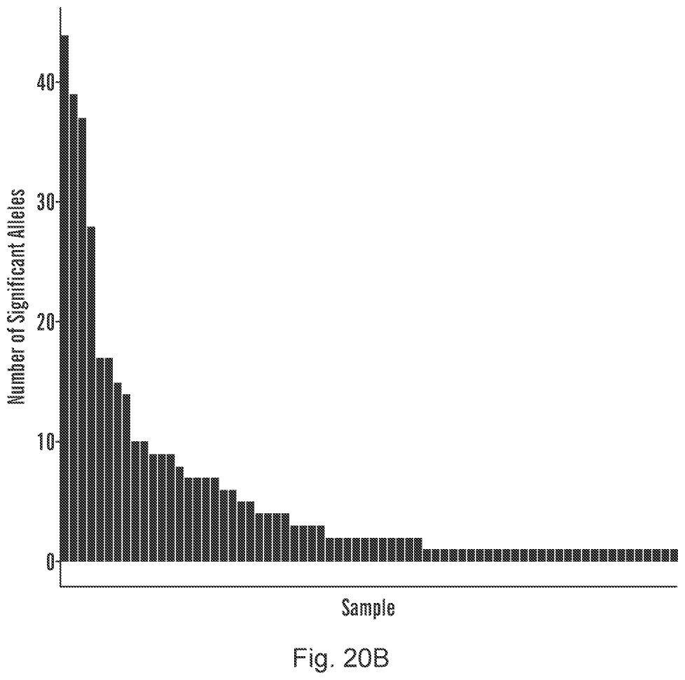

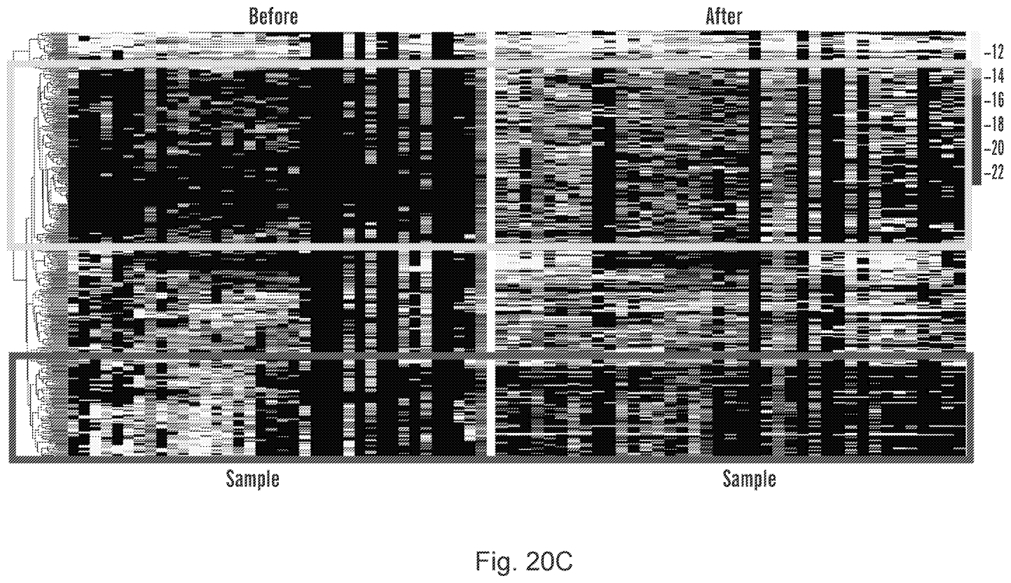

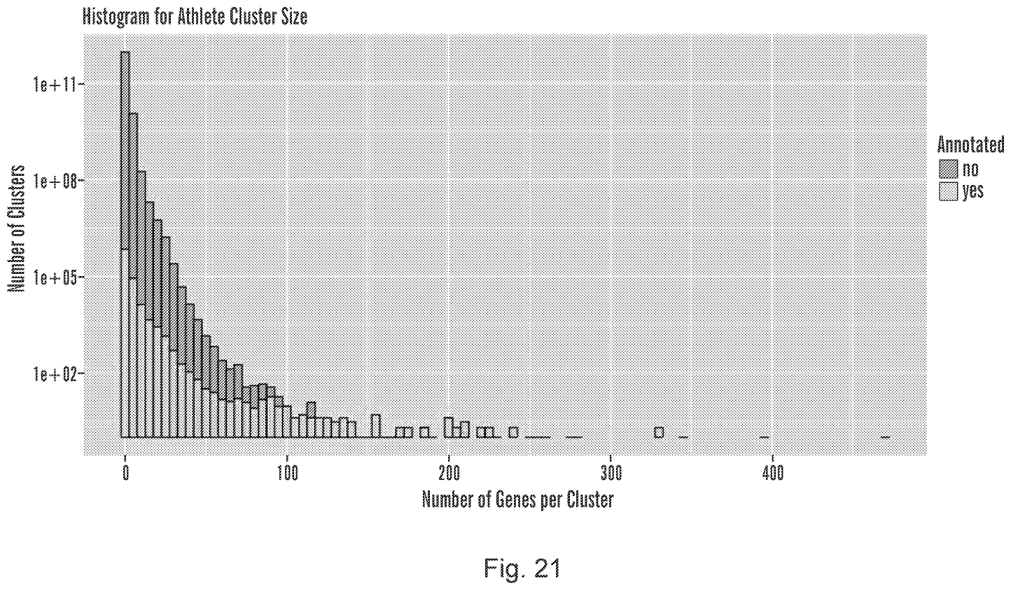

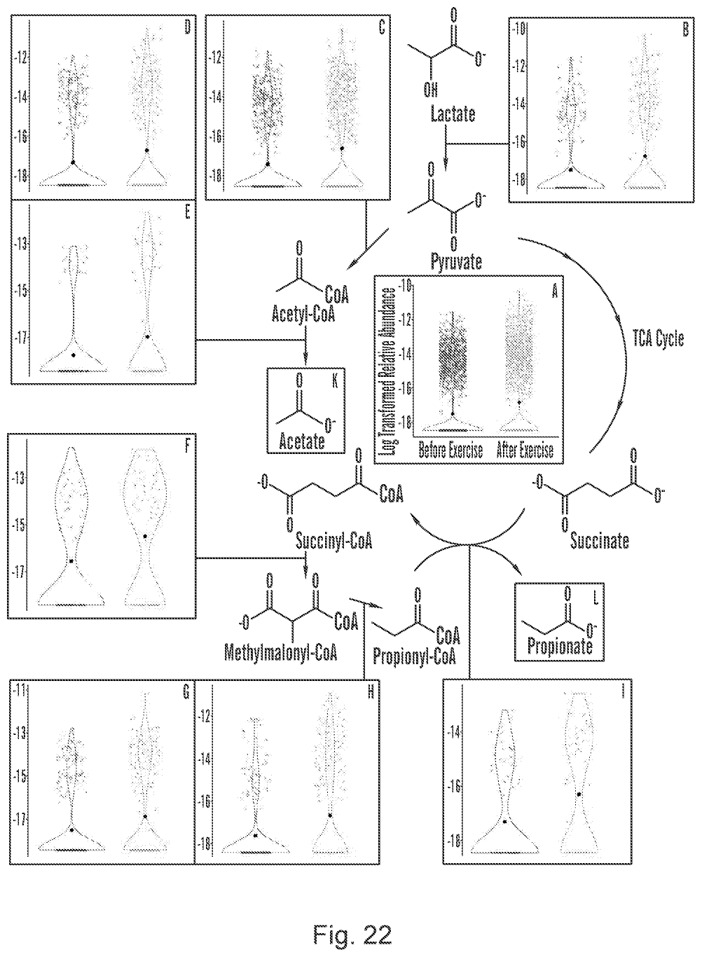

To test whether these results would be replicated in an independent cohort of athletes, shotgun metagenomic sequencing was performed on stool samples (n=87) from ultra-marathoners and Olympic trial rowers both before and after exercise. Putative taxonomic abundances reproduced the previous 16S sequencing-based association with Veillonella (see e.g., FIG. 20A-FIG. 20C). By utilizing novel algorithms that allow for construction of metagenomic gene catalogs at massive scale through efficient use of cloud computing, the phenotypic modulating effects of millions of microbial genes on athletes was investigated by building a sample (n=87) by gene (n=2,288,155) relative abundance matrix (see e.g., FIG. 21 and FIG. 22; Methods). The inability of Veillonella to ferment carbohydrates, coupled with high observed abundance of the lactate import permease in previously sequenced isolates, suggests that metabolic enzymes facilitating lactate breakdown are likely conserved. Across the entire ultramarathon and rower cohorts, there exist a group of gene families with differential relative abundance pre and post exercise (see e.g., FIG. 22) representing every step of the enriched methylmalonyl-CoA pathway (P=0.00147; Methods) degrading lactate into propionate as assigned by Enzyme Commission (EC) IDs (see e.g., FIG. 3A). Given the limited prevalence of the methylmalonyl-CoA pathway across lactate-utilizing microbes, this enrichment post-exercise implicates Veillonella in causing functional change in the metabolic repertoire of the gut microbiome. Indeed, strong production of acetate and propionate was verified by performing mass spectrometry on spent media collected after growing three Veillonella strains isolated from the human athletes (V. parvula, V. dispar, and V. atypica) in BHI media supplemented with DL-lactate and semi-synthetic lactate media (see e.g., Table 1; Methods).

TABLE 1
SCFAs detected in spent media after 48 hours of growth with the indicated strain.

| | Butyrate | Propionate | Pyruvate | Lactate | Acetate |
|---|---|---|---|---|---|
| | (μM) | (μM) | (μM) | (μM) | (μM) |
| V. atypica LM | 19 ± 0.3*** | 10611 ± 584** | 4 ± 0.4*** | 431 ± 536*** | 92432 ± 3129** |
| L. bulgaricus LM | 9 ± 0.5 | 3 ± 1.5 | 34 ± 2.7 | 737 ± 45 | 4985 ± 247** |
| LM alone | 11 ± 0.1 | 4 ± 0.5 | 41 ± 0.4 | 851 ± 6.6 | 1741 ± 12.2 |
| V. atypica BHIL | 69 ± 0.5* | 2286 ± 68* | n/a | n/a | 4149 ± 118* |
| BHIL Alone | 147 ± 4.1 | 160 ± 1.4 | n/a | n/a | 7557 ± 30 |

LM = semi-synthetic lactate media;
BHIL = brain-heart infusion media supplemented with sodium lactate;
n/a = not quantified.
Each bar represents mean ± SEM.
(n = 2-3).
(*P < 0.05; **P < 0.01; ***P < 0.001; compared with media control using Welch's t-test).

Veillonella species metabolize lactate into the SCFAs acetate and propionate via the methylmalonyl-CoA pathway. Lactate dehydrogenase (LDH), the enzyme responsible for the first step of lactate metabolism, is present in a phylogenetically diverse group of bacteria (see e.g., FIG. 3B). Querying microbial isolate strain genome annotations from NCBI show that unlike Veillonella atypica, many other microbes are theoretically capable of utilizing lactate through LDH, but do not possess the full pathway to convert lactate into propionate (see e.g., FIG. 3C). Other obligate anaerobes such as Anaerostipes caccae commonly produce butyrate as a metabolic byproduct of lactate fermentation (see e.g., FIG. 3C). Interestingly, Eubacterium hallii can also both ferment lactate and produce propionate, although unlike in Veillonella this occurs in two distinct pathways: acetate and lactate are converted into butyrate and hydrogen while 1,2-propanediol is converted into propionate. Of note, both the reference genomes on NCBI for Veillonella dispar and Veillonella parvula are not annotated to have the succinate-CoA transferase needed for propionate production to occur; however, this is an annotation error as production of propionate was validated via mass spectrometry on isolates of these species.

Taken together, these results show that not only is the genus Veillonella enriched in athletes after exercise, but the metabolic pathway for lactate metabolism into propionate is also enriched, which is a pathway relatively unique to the Veillonella among human gut bacteria. This result raised the possibility that systemic lactate resulting from muscle activity during exercise may enter the gastrointestinal lumen and become metabolized by Veillonella.

Serum Lactate Crosses the Epithelial Barrier into the Gut Lumen.

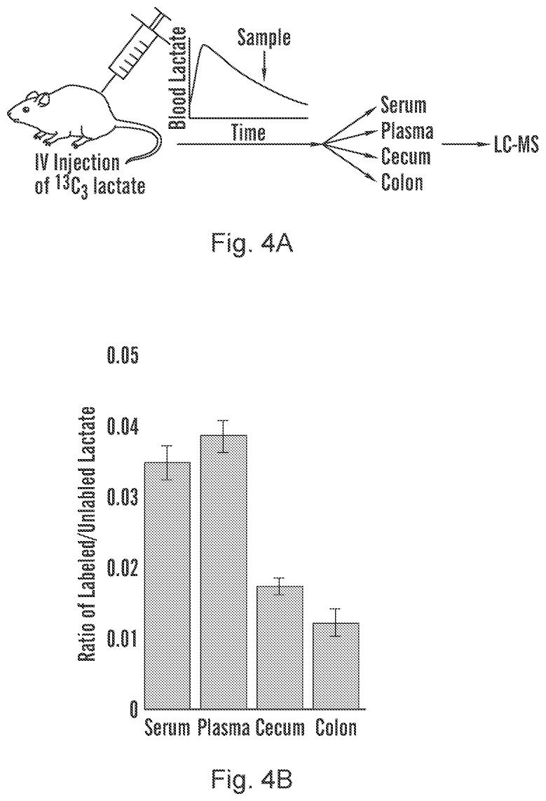

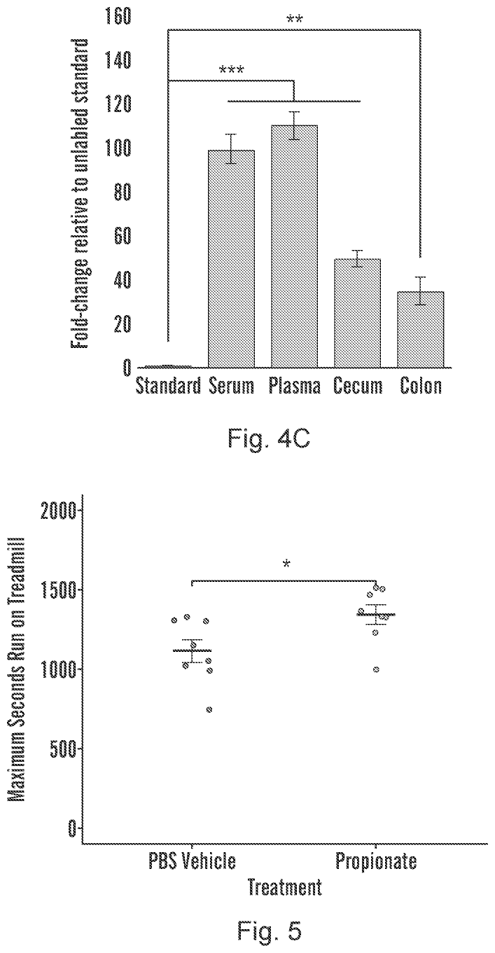

It was next determined whether systemic lactate is capable of crossing the epithelial barrier into the gut lumen. To investigate this, 100 μL of 400 mM ¹³C₃ sodium lactate was injected into the tail veins of mice colonized with either Veillonella atypica or Lactobacillus bulgaricus, and the mice were sacrificed 12 minutes after injection. This time-point was chosen because it was the earliest time at which serum lactate levels were observed to return to baseline levels after tail-vein injection. At sacrifice, serum and plasma were immediately collected following cardiac puncture, and intestinal luminal contents were collected by removing the colon and cecum from the mice and gently sampling the inner surface of the tissue. By performing liquid-chromatography and mass spectrometry (LC-MS) on these tissues, ¹³C₃-labelled lactate was present in both the serum and plasma as well as in the lumen of the colons and ceca (see e.g., FIG. 4). No ¹³C₃-labelled propionate was detected in these tissues; however, the 12 minute time-point from tail-vein injection to sacrifice is likely insufficient time for labelled lactate crossing the gut barrier to be metabolized into propionate by gut Veillonella.

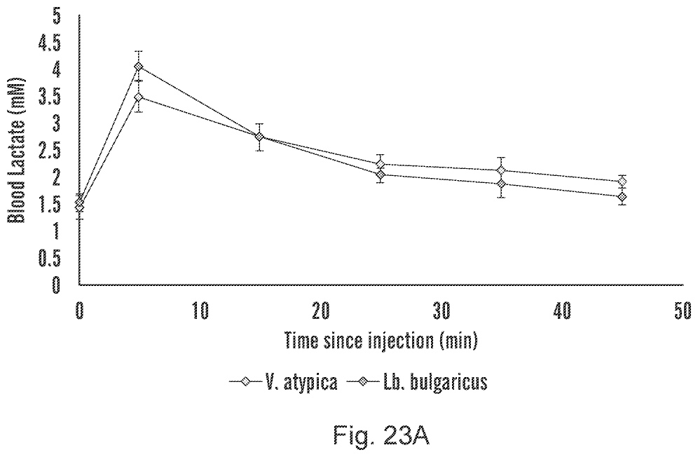

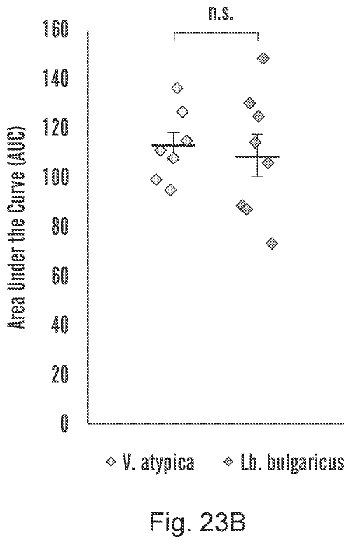

As serum lactate is capable of entering the intestinal lumen, it was next determined whether Veillonella colonization actively limits blood lactate levels by serving as a metabolic “sink.” To test the capability of Veillonella to accelerate blood lactate clearance in vivo, 750 mg/kg sodium lactate was injected intraperitoneally into mice treated with 10⁹ colony forming units (CFU) of either V. atypica or L. bulgaricus, and blood lactate was monitored over time. Neither the basal nor the peak lactate levels between the treatment groups were significantly different (see e.g., FIG. 23). The vast majority of lactate processing occurs in the liver, and although systemic lactate was observed infiltrating the intestinal lumen, Veillonella lactate metabolism did not detectably impact systemic lactate clearance.

### Colorectal Propionate Instillation is Sufficient to Enhance Treadmill Runtime.

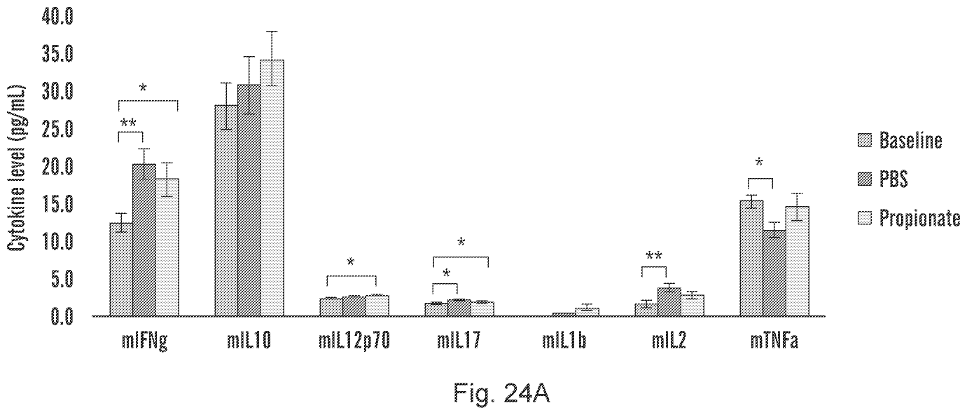

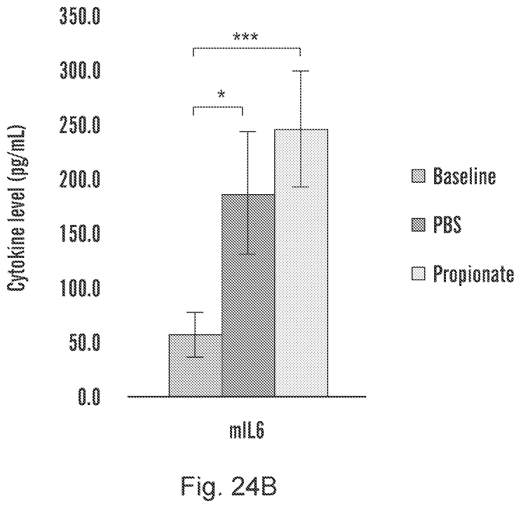

Propionate has been shown to increase heart rate, VO₂ max, and affect blood pressure in mice as well as raise the resting energy expenditure and lipid oxidation in fasted humans. To test whether the exercise-enhancing effects of Veillonella may be attributable at least in part to propionate, intrarectal instillation of Veillonella was performed in our mouse treadmill model. Propionate was introduced intrarectally rather than orally because colonic absorption via the pelvic plexus privileges propionate to direct systemic circulation, just as with Veillonella-produced propionate. Intrarectal propionate instillation (n=8) compared with pH-matched placebo vehicle (n=8) resulted in increased treadmill runtime similar to that of Veillonella atypica gavage (P=0.03, see e.g., FIG. 5). As in the Veillonella gavage experiments, a panel of inflammatory cytokines was run on serum taken 40 minutes after treadmill running, but no significant differences in cytokine levels were found (see e.g., FIG. 24A, FIG. 24B). Therefore, introduction of propionate to the colon alone is sufficient to result in an enhanced exercise phenotype.

### Discussion.

Coupling computational approaches, multi'omic data collection approaches, and experimental validation shows much promise as a method to approach unvalidated metagenomic associations. Acting on this principle, the following results were observed: 1) increased Veillonella abundance in the gut microbiome post-exercise in two independent cohorts of athletes; 2) a universal metagenomics overrepresentation of the Veillonella methylmalonyl-CoA pathway post exercise in athletes; 3) systemic lactate crossing the gut barrier into the lumen of the gut; 4) improved treadmill performance in mice in a longitudinal AB/BA crossover study in Veillonella-inoculated mice; and 5) improved treadmill performance of mice treated with propionate via intracolonic infusion.

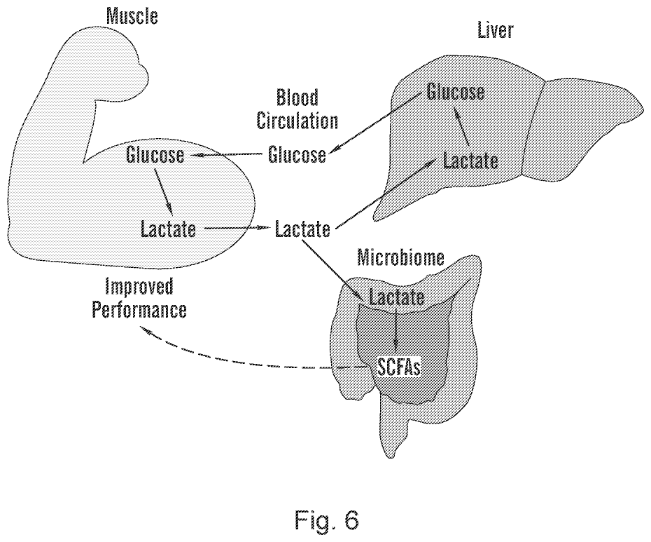

Without wishing to be bound by theory, these data illustrate a model in which systemic lactate produced during exercise crosses to the gut lumen and is metabolized by Veillonella into propionate in the colon, which in-turn serves to promote performance. It is contemplated that gut colonization of Veillonella augments the Cori cycle by providing an alternate lactate processing method where systemic lactate is converted into SCFAs that re-enter circulation (see e.g., FIG. 6). Indeed, SCFAs are absorbed in the sigmoid and rectal region of the colon as it enters the pelvic plexus, bypassing the liver and draining via the vena cava to reach systemic circulation directly. Thus, it is contemplated that microbiome-derived SCFAs augment performance directly and acutely, and that lactate generated during sustained bouts of exercise is accessible to the microbiome and is converted to these SCFAs that improve athletic performance.

In conclusion, the microbiome is a critical component of physical performance and the benefit derived from it. An important question is how this performance-facilitating organism has come to be more prevalent amongst athletes in the first place. While not wishing to be bound by theory, it is likely that the high-lactate environment of the athlete provides a selective advantage for colonization by lactate metabolizing organisms such as Veillonella. To the extent that the ability to metabolize lactate to propionate relates to the ability to enhance exercise endurance, it is surprising that there is an apparent preference for Veillonella, as opposed to the many other lactate-metabolizing organisms. Veillonella in the physically active host therefore serves as a key example of a symbiotic relationship in the human microbiome.

### Materials and Methods

### Code Availability Statement

Unless otherwise noted, all plots were generated in R version 3.4.1 with the ggplot2, dplyr, scales, grid, and reshape2 packages. Large scale data analysis was done on AWS utilizing machines running Ubuntu 16.04. Data curation methods were coded in python version 2.7.12. Unless otherwise noted specifically in the rest of the methods section, code utilized is available on the worldwide web at the following addresses: github.com/kosticlab/athlete and github.com/kosticlab/aether.

### Metaphlan2 Taxonomy in Metagenomics Data

Putative taxonomic abundances were calculated with Metaphlan2 and found to have the same association between Veillonella and exercise status as the previous marathon runner results (p=0.03; see e.g., FIG. 20A).

### Annotations

To compare trends in the aggregate microbiome with the metabolic processes of microbes that had elevated 16S abundance in the prior experiment, a pairwise ANOVA was performed on all ˜2.3M genes in the catalog to look for significant differences before and after exercise. 396 gene families with unique annotations showed statistically different relative abundance (p<0.005). While FDR correction did not yield significant individual genes, of these 396 gene families, 391 shared functional annotations with the reference assemblies of the V. atypica type strain on NCBI. Of the significant genus level results from the 16S data, Veillonella had extremely high quality assemblies of cultured isolates.

Significant alleles were present in each of the 87 samples (e.g., FIG. 20B). Interestingly, when all 396 significant alleles were segregated by exercise state and sample, discordant shifts of relative abundance were observed (e.g., FIG. 20C). Changes in global microbiome function were associated with Veillonella abundance, and conserved Veillonella genes generally played metabolic roles.

### 16S Analysis

Each subject in the study provided fecal samples on a daily basis, up to one week before and one week after the marathon (controls did not run in the marathon but provided fecal samples). Genomic DNA were extracted from these samples and 16S rDNA amplicon sequencing was performed followed by bioinformatic analysis to obtain genus level resolution of bacteria in each individual's microbiome.

16S reads were processed with the dada2 pipeline and phyloseq. Default settings were used for filtering and trimming. Built in training models were utilized to learn error rates for the amplicon dataset. Identical sequencing reads we re-combined through dada2's dereplication functionality, and the dada2 sequence-variant inference algorithm was applied to each dataset. Subsequently, paired end reads were merged, a sequence table was constructed, taxonomy was assigned, and abundance was calculated at all possible taxonomic levels.

### 16S Mixed Effect Modeling

A series of generalized linear mixed effect models (GLMMs) was constructed to predict Veillonella relative abundance in the marathon participants from both random effects (individual variation per athlete that manifests longitudinally) and fixed effects (USDA MyPlate consumption categories, protein powder supplementation, menstruation status, race, time, body mass index (BMI), weight, gender, and age).

16S Veillonella relative abundance modeling for athletes participating in the marathon was done with the R nlme package. 1,267 meal records logging every instance of food consumption over the course of the study were quantified according to USDA MyPlate™ and associated with daily 16S samples by a nutritionist. Relative abundance was first modeled as the following formula:

Abundance=β₀+β_{Time}+β_{Sex}+β_{Weight}+β_{BMI}+β_{Age}+β_{Race}+β_{Menstration}+β_{Vegetables}+β_{Fruits}+β_{Grains}+β_{Protein}+β_{Dairy}+β_{Dietary} Protein Supplementation

Subsequently, a second model was generated that included interaction terms of Time:Vegetables and Time:Menstruation. Significance was calculated for all coefficients included in the GLMM with Wald Z-tests (default calculation in the library utilized). Coefficients were created with the coefplot2 package.

The code for the two models is provided below.

model_1<−1me(Veillonella˜Time+Sex+Weight+BMI+Age+Race+Menstruation+vegetables+fruits+grains+protein+dairy+dietary_protein_supp,random=˜1|SubjectID,data=marathon1 6S)

model_2<−1me(Veillonella˜Time+Sex+Weight+BMI+Race+Menstruation+vegetables+fruits+grains+protein+dairy+dietary_protein_supp+Time:vegetables+Time:Menstruation,rando m=˜1|SubjectID, data=marathon16S)

Model predictions overlaid on the underlying data were visualized with the ggplot2 R package. Detailed information about all coefficients and their correlation for the model utilized in the figure is shown in FIG. 7.

Model results were validated with both leave-one-out cross-validation (LOOCV) and permutation testing on shuffled labels.

### Treadmill Runtime Mixed Effect Modeling

Despite the high number of mice utilized in the AB/BA crossover experiment, comparisons of raw runtime in this context could be confounded both by carryover effect (modeled as a sequence effect) inherent in the longitudinal study design as well as unavoidably high inter-mouse variation. To account for this, a series of GLMMs were constructed predicting runtime (methods). These models incorporate both random effects (individual variation per mouse that manifests longitudinally) and fixed effects (treatment day, treatment sequence, and treatment given). Modeling was conducted with the R nlme package. Visualization of coefficients was conducted using the coef2plot R package. Visualization of predictions overlaid on data was conducted using the R ggplot2 package.

Models were constructed to predict treadmill runtime in the AB/BA crossover experiment to include treatment effect of Veillonella, period effects (time of treatment), carry-over effects due to the treatment crossover, and effects for naturally occurring mouse variation. In general, expected runtime is modeled in Table 2.

TABLE 2
Expected Runtime Model, where α_{A} and α_{B} are
treatment effects, λ_{A} and λ_{B} are carry-over effects,
and π₁ and π2 are period effects.

| | Week 1 | Week 2 |
|---|---|---|
| Sequence | μ + π₁ + α_{A} | μ + π₂ + α_{b} + λ_{A} |
| V. atypica -> L. bulgaricus | | |
| Sequence | μ + π₁ + α_{B} | μ + π₂ + α_{A} + λ_{B} |
| L. bulgaricus -> V. atypica | | |

Carry-over effect was initially modeled as a sequence effect or a period-specific treatment effect (interaction term). The R code for the models is provided below: model_1<−1me(seconds_run˜treatment+sequence+period,random=˜1subject,data=datain) model_2<−1me(seconds_run˜Treatment*period,random=˜1|subject,data=datain)

By gauging correlation of coefficients, model_1 was selected for the figure in the paper. Regression estimates for coefficients are provided in FIG. 9.

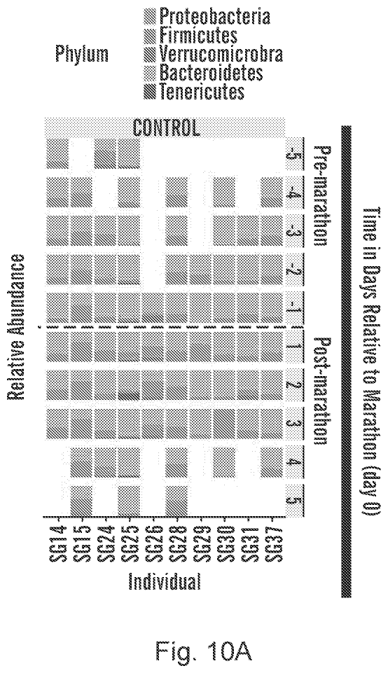

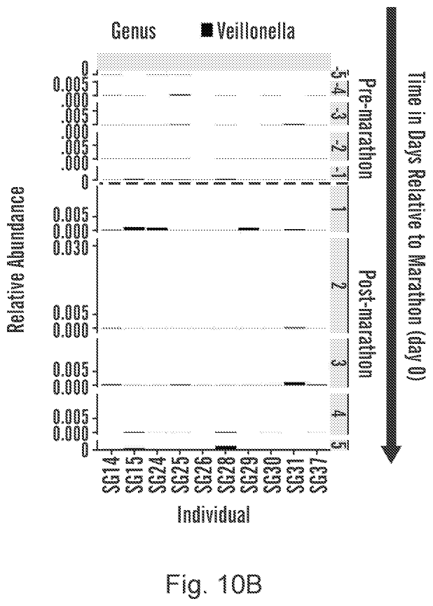

Detailed information about all coefficients and their correlation for the model utilized in the figure is provided in FIG. 10.

Model results were validated with both LOOCV and permutation testing on shuffled labels.

### Comparative Genomes

Genome annotations were retrieved from NCBI reference genomes. Phylogenetic trees were generated from NCBI taxonomy and visualized with phylo.io. Heatmaps were generated with the pheatmap package in R.

### Metagenomic Analysis

All steps in the processing of raw metagenomic data were done utilizing the Aether package. Raw reads were de novo assembled using megahit. Open reading frames and annotations were generated using prokka. A gene family catalog was generated from the called open reading frames at 95% identity utilizing the CD-HIT software package. A raw abundance count matrix was generated utilizing the gene family catalog, bowtie2, and samtools. The raw abundance count matrix was normalized both by sample and by gene length. Metabolic pathways were queried using MetaCyc and EC IDs were pulled from prokka annotations. R was utilized to perform the majority of statistical tests with the exception of pairwise ANOVA tests, for which the SciPy library in python was used. Root mean square error calculations were performed using the plotrix package.

### Gene Catalog Creation

Raw reads were processed and de novo assembled into 4,802,186 contigs. 4,792,638 total Open Reading Frames were called, which were subsequently clustered into 2,288,155 gene families with a threshold of 95% identity to create a gene catalog alongside putative annotations assigned by homology. Of these gene families, 801,307 were assigned annotations, and 1,486,948 were putatively classified as hypothetical proteins. Comparing annotation state versus gene family size yields the expected result that larger families, which are likely to be present in more microbes, tend to have many more annotations (see e.g., FIG. 14). Raw reads were then aligned back to the gene catalog to create a raw count abundance matrix. This matrix was normalized both per sample and by gene length to create a relative abundance matrix.

### Pathway Elucidation

Reactions involved with the breakdown of lactate to both propionate and acetate were manually associated with EC IDs using MetaCyc.

### Participation Recruitment

All study participants were recruited following an IRB approved Sports Genomics protocol. Each participant read and signed a consent form prior to study enrollment.

### Sample Collection, Extraction, and Library Preparation

As collection materials, study participants were provided a 15 ml falcon tube with a 1 ml pipette tip inserted inside. Participants were instructed to dip pipette tips into soiled toilet tissue then place back into tubes and label with date and time of collection. Samples were kept at 4° C. for short term storage until sample pickup, at which time they were immediately placed onto dry ice, then transferred to a −80° C. freezer for long term storage.

Fecal samples were thawed on ice and resuspended into 2-5 ml of Phosphate Buffered Saline, of which 250 μL was used for DNA extraction using the MOBIO™ Power Soil high throughput DNA extraction kit, following manufacturer's protocol. For 16s rDNA library construction, 1-5 μL of purified DNA was used for PCR amplification of the V4 variable region using the Q5 hotstart polymerase™ (NEB™). Primers were adapted from the Earth Microbiome Project™ (available on the world wide web at earthmicrobiome.org), attaching illumina PE adaptors (Forward: SEQ ID NO: 1 CTT TCC CTA CAC GAC GCT CTT CCG ATC TGT GCC AGC MGC CGC GGT AA; Reverse: SEQ ID NO: 2 GGA GTT CAG ACG TGT GCT CTT CCG ATC TGG ACT ACH VGG GTW TCT AAT). Illumina barcodes were added to libraries during a second PCR step (Forward: SEQ ID NO: 3 AAT GAT ACG GCG ACC ACC GAG ATC TAC ACT CTT TCC CTA CAC GAC GCT C; Reverse: SEQ ID NO: 4 CAA GCA GAA GAC GGC ATA CGA GAT GTG ACT GGA GTT CAG ACG TGT GCT C), and end products were purified using Zymogen™'s clean and concentration kit. Individual libraries were quantified and normalized for sequencing using the Quant-It Picogreen™ reagent (Thermo Fisher™). For whole genome shotgun library construction, 1 ng of purified DNA was used for Illumina's Nextera XT Tagmentation™ kit, following manufacturer's protocol. Libraries were submitted to a biopolymers core sequencing facility for bioanalyzer QC and 150 b.p. paired-end sequencing reads using either Illumina miseg™ or Hiseq 2500™ (high output mode) for 16s rDNA and shotgun analysis, respectively.

### Metadata Collection

Each study participant was provided a questionnaire to collect health, dietary, and athletic background information (adapted from The American Gut™, available on the world wide web at americangut.org). Additionally, for each sample collection, study participants filled out a daily annotation sheet to collect dietary, exercise, and sleep information.

### Preparation of Bacteria for Gavage

Veillonella atypica and Lactobacillus bulgaricus were grown in 250 mL BHI broth supplemented with lactate (10 mL 60% sodium lactate/L) and MRS broth, respectively. OD 600 was monitored and at OD 0.4-0.6, cells were pelleted by refrigerated centrifugation at 5,000 g for 10 min. The pellet was washed in PBS and resuspended in 2 mL residual PBS. 100 μL aliquots were frozen at −80° C. and CFU/mL measured by serial dilution onto BHI lactate agar plates.

Veillonella atypica was gavaged in wild-type C57BL/6 mice to determine viability and transit time through the GI tract, observing peak viable bacterial CFU counts in fecal pellets 5 h after gavage.

### Treadmill Crossover Experiment

For treadmill experiments, 8-12 week old CL57BL/6 mice (n=32) were acclimated to treadmilling with 2 bouts of 30 minutes of 5 m/min walking, split over 2 consecutive days. For exhaustion measurements, mice were fasted for 100 minutes prior to gavage with 200 μL of 2.5% sodium bicarbonate to neutralize stomach contents. 20 minutes after the first gavage, mice were gavaged 200 μL of either V. atypica or L. bulgaricus, prepared as above and normalized to 5×10⁹ CFU/mL. 5 hours post gavage, mice were run on the treadmill, starting at 5 m/min and increasing speed by 1 m/min every minute until exhaustion. Time of exhaustion was recorded for every animal, defined as a mouse failing to return the treadmill from the rest platform after three consecutive attempts to continue running This protocol was repeated for 2 more days, followed by 4 days of rest and 3 days of crossover treatment. On the first day of treatment, serum was collected 40 minutes post-exhaustion via tail-vein bleed and measured using the Ciraplex™ multiplex mouse cytokine assay (Aushon BioSystems™)

### In Vitro Growth and SCFA Analysis

Veillonella species (V. dispar, V. parvula, and V. atypica) were isolated and purified from several study participants and grown in three different media compositions: 1) Brain Heart Infusion Broth (BHI) supplemented with lactate (10 mL 60% sodium lactate/L); 2) MRS broth (BD) supplemented with lactate (10 ml 60% sodium lactate/L); 3) Semi-synthetic lactate medium (per liter: 5 g bacto yeast extract, 0.75 g sodium thioglycolate, 25 ml basic fuchsin, 21 ml 60% sodium lactate, pH 7.5). Veillonella species were inoculated into each medium, under anaerobic conditions and allowed to grow for 48 h to reach stationary phase. After 48 h, bacteria were pelleted, and supernatants were collected for lactate and SCFA measurements. Approximately 10 μl of supernatant was used to measure lactate via the Lactate Scout™ meter (available on the world wide web at Lactate.com). The remaining supernatants were frozen at −80° C. and then submitted to the a small molecule mass spectrometry core facility for butyrate, propionate, and acetate quantitative analysis.

SCFAs identified from the mass spectrometry in all three media conditions corresponded with the propionate end product suggested by the metagenomic results. Acetate was not observed in MRS or BHI, likely due to high existing concentrations in the media making the forward reaction thermodynamically unfavorable. However, acetate production was observed in semi-synthetic lactate media.

¹³C₃-Lactate Flux Tracing

10 week old C57BL/6 mice were treated with sodium bicarbonate followed by 10⁹ CFU of either Veillonella atypica or Lactobacillus bulgaricus, prepared as above (n=3;4). 20% w/w ¹³C₃ sodium lactate (Cambridge Isotope Laboratories™) was diluted with PBS to a concentration of 400 mM in PBS. Mice were injected with 100 μL intravenously via the tail vein and after 9 minutes anaesthetized with isoflurane. One mouse treated with Veillonella atypica was unable to be injected due to vein clamping and had to be removed. 10 minutes post-injection, anesthetic was confirmed via foot pinch and mice were sacrificed via cardiac puncture. Whole blood was divided into two samples to obtain both serum and plasma, which was flash frozen in liquid nitrogen and stored at −80° C.

Immediately following cardiac puncture, mice were dissected to remove colon and cecum, and the contents were removed by squeezing with sterilized forceps into pre-weighed tubes. Contents were immediately flash frozen in liquid nitrogen. Timing varied slightly, between 17 and 19 minutes post-injection.

Samples were analyzed for lactate and propionate by a Metabolomics Platform. LC-MS metabolomics were performed as previously described. 23 LC-MS traces were identified and integrated to quantify presence of ¹³C₀- and ¹³C₃-lactate isotopes.

### Lactate Clearance

To measure lactate clearance rate, mice were first fasted for 7 hours prior to measurement to stabilize basal lactate levels. 5 hours prior to measurement, mice were treated with sodium bicarbonate followed by 10⁹ CFU of either Veillonella atypica or Lactobacillus bulgaricus, prepared as above (n=8). 30 minutes prior to measurement, mice were weighed, individually caged, and a baseline blood lactate reading was taken using a Lactate Scout™ meter. Mice were administered sodium lactate via IP injection with a dosage of 750 mg/kg, prepared as a 75 mg/mL solution of sodium lactate in pH7.0 PBS. Blood lactate was monitored with a Lactate Scout™ meter at 5, 15, 25, 35, and 45 minutes post-injection.

### Colorectal Propionate Instillation

Treadmilling followed the same protocol as above. Mice were fasted 7 hours prior to exercise, to normalize metabolic profiles. 30 minutes prior to exercise, mice were treated with 200 μL of either PBS vehicle alone (n=8) or 150 mM sodium propionate in PBS (n=8) using a flexible gavage needle to introduce 200 μL of solution into the colon. Mice were run to exhaustion as above. This protocol was repeated for 3 consecutive days. On the first day of treatment, serum was collected 40 minutes post-exhaustion via tail-vein bleed and measured using the Ciraplex™ multiplex mouse cytokine assay (Aushon BioSystems™)

### Statistics

FIG. 1A and FIG. 1B: Wilcoxon rank sum test with continuity correction were used to look at differences in taxonomic composition before and after exercise. Mean Veillonella abundance was 0.9 orders of magnitude greater 1 day post exercise compared to 1 hour prior to exercise.

FIG. 1C and FIG. 1D: Longitudinal data was modeled with a GLMM approach. In this model, the random effect was individual variation per marathon runner. Fixed effects are shown in FIG. 1D. An advantage of this type of statistical analysis is that it can account for the large variation between marathon participants in this type of study.

To determine statistical significance, a Wald Z-test was used to assign p-values to coefficients in the GLMM. No outliers were removed in this analysis.

FIG. 2A: Each animal was treated with both Veillonella atypica and Lactobacillus bulgaricus as part of the AB/BA crossover. Because all 32 animals were treated twice and compared between treatments, p-value was generated using a paired t-test (p=0.022).

FIG. 2B: Longitudinal data was modeled with a GLMM approach. In this model, the random effect was individual variation per mouse. Fixed effects were treatment effect, period effect (at what time point measurements were made), and carryover/sequence effect (if the order of treatments in the crossover affected later results). An advantage of this type of statistical analysis is that it can account for the large variation between mice in this type of study.

FIG. 2B shows seconds run until exhaustion at 6 timepoints, with each of the 32 mouse having one measurement per time point. For each treatment order (LLLVVV and VVVLLL) the GLMM was fitted both to each individual mouse (skinny blue and red lines; note that these are all parallel for mice in the same treatment order—the space between these lines represents the “random effect” of natural variation between mice) and all mice (Population) with the same treatment order (thick blue and red lines).

To determine statistical significance, a Wald Z-test was used to assign p-values to coefficients in the GLMM. No outliers were removed in this analysis.

FIG. 3A and FIG. 15: p-values for individual genes were generated utilizing pairwise ANOVA comparing before and after exercise relative abundance. Non-significant families were associated with homologs common in other microbes that do not change in abundance. To determine the significance of potential overrepresentation, 1,000 global EC IDs were randomly selected and mean difference in relative abundance between samples taken before and after exercise. These EC IDs were used to construct an odds table to determine the probability of having a set of 8 selected EC IDs with increases in mean gene-level relative abundance after exercise. This calculation determined that the relative abundances changes in FIG. 3B-FIG. 3I are significant (p=0.00147 using a Fisher's Exact Test for count data).

Table 1: p-values were generated using Welch's t-test (unequal variances t-test).

FIG. 4C: p-value was generated using a one-sample t-test. Ratios of labeled/unlabeled lactate from samples were compared to the expected ratio determined mathematically. Each sample was independently compared to the expected ratio, then multiple hypothesis correction was performed using the FDR correction method of Benjamini & Hochberg (1995). (Serum p=0.00001; plasma p=0.00001; cecum content p=0.00001; colon content p=0.001).

FIG. 5: p-value was generated using Welch's t-test (unequal variances t-test). (p=0.028).

For further information, see e.g., Scheiman et al., Meta-omics analysis of elite athletes identifies a performance-enhancing microbe that functions via lactate metabolism, Nat Med. 2019 July;25(7):1104-1109; International Patent Application WO2017180501; US Patent Application US20190160118; the contents of each of which are incorporated herein by reference in their entireties.

1. A composition comprising at least one Veillonella probiotic bacterium and an acceptable excipient, diluent, or carrier.

2. The composition of claim 1, wherein the Veillonella probiotic bacterium is selected from the group consisting of Veillonella atypica, Veillonella dispar, and Veillonella parvula.

3. The composition of claim 1, wherein the Veillonella probiotic bacterium comprises Veillonella atypica, Veillonella dispar, and Veillonella parvula.

4. The composition of claim 1, wherein the Veillonella probiotic bacterium comprises and expresses genes encoding enzymes:
(a) sufficient to metabolize the conversion of lactate into short-chain fatty acids comprising one or more of propionate and acetate; or
(b) from the methylmalonyl-CoA pathway.

5. (canceled)

6. The composition of claim 1, wherein the Veillonella probiotic bacterium is viable and lyophilized.

7. The composition of claim 1, wherein the acceptable excipient, diluent, or carrier comprises water, saline, dextrose, glycerol, ethanol or the like and combinations thereof.

8. The composition of claim 1, wherein the composition is in the form of a pill, a tablet, or a capsule.

9. A method of enhancing exercise endurance, comprising administering an effective dose of the composition of claim 1 to a subject in need thereof.

10. The method of claim 9, wherein the composition is administered via an oral, enteric, gastrointestinal, rectal, or parenteral route.

11. The method of claim 9, wherein the subject is a human athlete or human in need of enhanced exercise endurance.

12. (canceled)

13. A device preloaded for administration to a body cavity comprising an effective dose of propionate to enhance exercise endurance.

14. The device of claim 13, wherein the propionate comprises 10 mM-1000 mM sodium propionate.

15. The device of claim 13, comprising a suppository.

16. The device of claim 13, wherein the body cavity is the rectum.

17. A method of enhancing exercise endurance comprising administering to a subject in need thereof an effective amount of propionate via a device of claim 13.

18. (canceled)

19. The method of claim 17, wherein the propionate is administered via a rectal, intracolonic, gastrointestinal, enteric, oral, or parenteral route.

20. The method of claim 17, wherein the subject is a human athlete or human in need of enhanced exercise endurance.

21. An engineered-probiotic bacterium that is effective at enhancing exercise endurance.

22. The engineered probiotic bacterium-of claim 21, wherein the engineered probiotic bacterium comprises and expresses genes encoding enzymes:
(a) sufficient to metabolize the conversion of lactate into short-chain fatty acids comprising one or more of propionate and acetate;
(b) from the methylmalonyl-CoA pathway; or
(c) selected from the group consisting of: Acylphosphatase/Phosphate Acetyltransferase, Fumarate Hydratase, Fumarate Reductase, Lactate Dehydrogenase, Malate Dehydrogenase, Methylmalonyl-CoA Carboxyltransferase, Methylmalonyl-CoA Epimerase, Methylmalonyl-CoA Mutase, Pyruvate:Ferredoxin Oxidoreductase, Pyruvate Carboxylase, Pyruvate Dehydrogenase, Succinate-CoA Transferase, and Succinate Dehydrogenase.

23.-25. (canceled)

26. The composition of claim 1, wherein the composition is a food, beverage, or dietary supplement composition.

27.-30. (canceled)
